(19) United States
(12) Patent Application Publication
(10) Pub. No.: US 2025/0302762 A1
(43) Pub. Date: Oct. 2, 2025

(54) NANOMATERIAL DELIVERY VEHICLE AND METHOD OF USE THEREOF

(71) Applicant: University of Connecticut, Farmington, CT (US)

(72) Inventors: Yupeng Chen, Sharon, MA (US); Wuxia Zhang, Willimantic, CT (US); Jinhyung Lee, Willimantic, CT (US)

(73) Assignee: University of Connecticut, Farmington, CT (US)

(21) Appl. No.: 18/852,925

(22) PCT Filed: Mar. 31, 2023
(86) PCT No.: PCT/US2023/065193

§ 371 Date: Sep. 30, 2024

Related U.S. Application Data
(60) Provisional application No. 63/326,462, filed on Apr. 1, 2022.

(51) Int. Cl. A61K 9/51; B82Y 5/00

(52) U.S. Cl. CPC A61K 9/5123; B82Y 5/00

(57) ABSTRACT

A self-assembled nanomaterial includes a Janus base nanotube, wherein the Janus base nanotube includes at least one compound represented by Formulas I to XII, or a pharmaceutically acceptable salt thereof. Also described are compositions including the Janus base nanotubes.

### CROSS-REFERENCE TO RELATED APPLICATIONS

The present disclosure claims priority to and the benefit of International PCT Application No. PCT/US2023/065193, filed 31 Mar. 2023, and U.S. Provisional Application 63/326,462, filed 1 Apr. 2022 and titled NANOMATERIAL DELIVERY VEHICLE AND METHOD OF USE THEREOF, which is incorporated by reference herein in its entirety for all purposes.

### FEDERAL RESEARCH STATEMENT

This invention was made with government support under W81XWH-21-1-0274 awarded by the Medical Research and Development Command, AR072027 awarded by the National Institutes of Health, and 1905785, and 2025362 awarded by the National Science Foundation. The government has certain rights in the invention.

### INCORPORATION BY REFERENCE

In compliance with 37 C.F.R. 1.52(e)(5), the sequence information contained in electronic file name: EASC004PCTUSseglist1.5-12-2025_ST26.xml; size 89.5 KB; created on: 12 May 2025, is incorporated herein by reference in its entirety.

### BACKGROUND

Numerous methods of delivering biologically active materials have been developed and vary based upon the type and the target of the biologically active material. Examples of various delivery vehicles which have been used, particularly for RNA delivery, include virus vectors, lipid nanoparticles, cationic polymers, liposomes, polymer nanoparticles, carbon nanoparticles, superparamagnetic iron oxide nanoparticles (SPION), gold nanoparticles, silver nanoparticles, metal-organic frameworks, cell-penetrating peptides, black phosphorus nanosheets, and DNA nanostructures. Delivery vectors such as lipid nanoparticles can deliver RNAs, but a low endosomal escape has been reported, which reduces efficacy. Moreover, the cationic lipid content of lipid nanoparticles tends to drive a pro-inflammatory phenotype. These limitations make it challenging to obtain high efficacy RNA delivery for therapeutic applications and have impeded translation into clinics.

There remains a need for improved delivery of biologically active materials such as small molecules, nucleic acids, proteins and gene silencing or gene editing tools, and/or other large molecule biological materials.

### SUMMARY

Disclosed herein is a self-assembled nanomaterial comprising a Janus base nanotube (JBNT), wherein the Janus base nanotube comprises at least one compound represented by Formulas I to XII, or a pharmaceutically acceptable salt thereof:

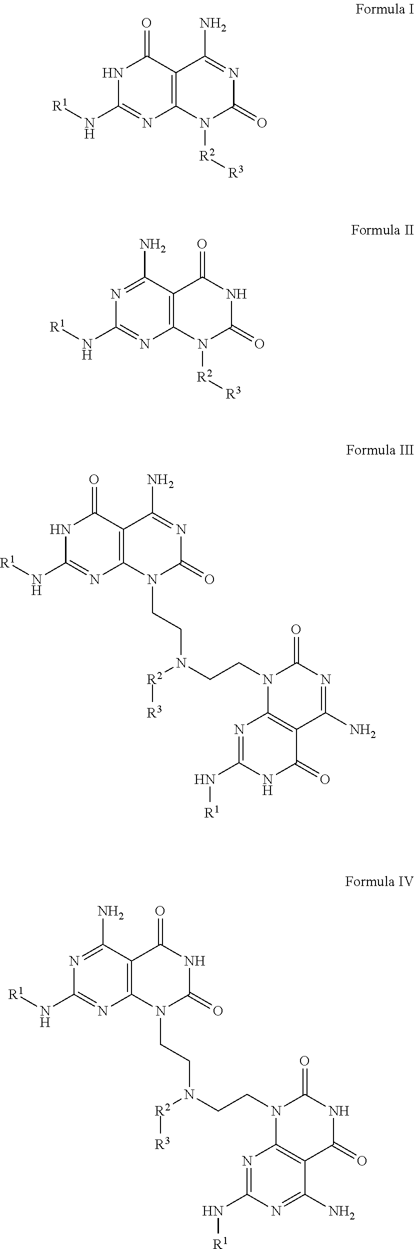

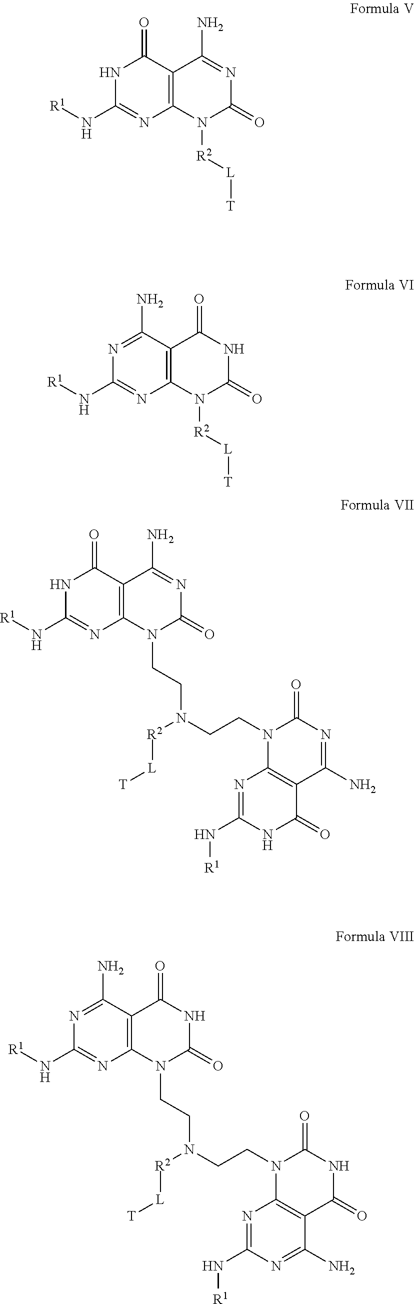

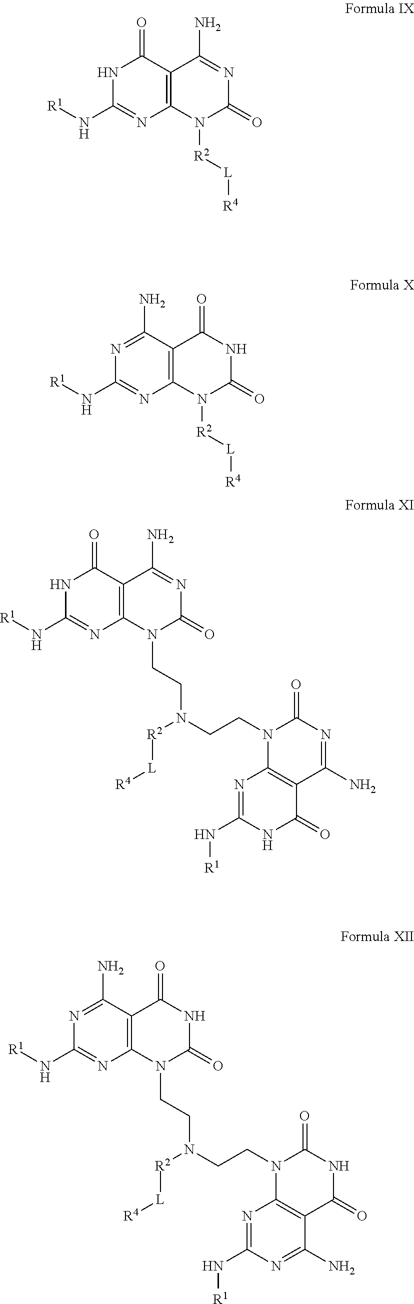

wherein,
R¹ is H or CH₃;
R² is (CH₂)ⱼ, (CH₂CH₂O)ₖ, or (CH₂CH₂NH)ₘ, where j, k and m are independently an integer from 1 to 200;
R³ is an α-amino acid, a β-amino acid, an α-polypeptide, or a β-polypeptide;
L is a bond or a linker group;
T is a biologically active molecule or a targeting molecule/moiety; and
R⁴ is a coating material.

Also disclosed herein are injectable compositions comprising the self-assembled nanomaterials described above and a pharmaceutically acceptable carrier.

The above described and other features are exemplified by the following figures and detailed description.

### BRIEF DESCRIPTION OF THE DRAWINGS

The following figures are exemplary embodiments wherein the like elements are numbered alike.

FIGS. 1A, 1B, 1C, and 1D show targeting peptide—JBNP for active targeting. FIG. 1A is a schematic illustration of a LysJBNP-targeting peptide; FIG. 1B shows the structure of targeting peptide-LysJBNT; FIG. 1C includes TEM images of LysJBNP without targeting peptide and LysJBNP with targeting peptide RLDPTSYLRTFWC; FIG. 1D is a graph of absorption versus wavelength (nm) showing the ultraviolet-visible (UV-Vis) targeting of peptide—Lys-JBNT.

FIGS. 2A, 2B, 2C, 2D, 2E, 2F, 2G, and 2H show LysJBNP assembly and delivery. FIG. 2A is a schematic drawing of LysJBNP delivery; FIG. 2B is a graph shown zeta potential analysis; FIG. 2C is a graph showing UV-VIS analysis; FIG. 2D shows TEM images of the LysJBNPs; FIG. 2E shows the results of a gel retardation assay; FIG. 2F is CLSM z-stack images of siRNA-AlexaFluor®-488 delivered by the LysJBNPs; FIG. 2G are images showing inhibition of LysJBNP uptake; and FIG. 2H is a graph illustrating quantitative analysis of JBNP uptake by C28/I2 human chondrocytes cells.

FIGS. 3A, 3B, 3C, 3D, 3E, 3F, 3G, 3H, 3I, 3J, 3K, and -3L show Arg-JBNP assembly and delivery. FIG. 3A is TEM image of Arg-JBNP-miR140, Arg-JBNP-Cas9mRNA, and Arg-JBNP-albumin; FIG. 3B shows TEM image analysis of the size and width of Arg-JBNP-miRNA; FIG. 3C shows zeta potential analysis of Arg-JBNP-miR140; FIG. 3D shows a gel retardation assay; FIG. 3E shows a zeta potential analysis of Arg-JBNP-Cas9mRNA; FIG. 3F shows a CLSM z-stack images of siRNA-AF488 delivered by the Arg-JBNPs; FIG. 3C shows a flow cytometry analysis; FIG. 3H shows fluorescence images of Cy5.5-eGFP-mRNA delivery via Arg-JBNP or Lipo; FIG. 3I shows fluorescence images of BSA-AF488 delivery via Arg-JBNP; FIG. 3J shows time-dependent delivery of Cas9eGFP; FIG. 3K shows fluorescence images of Arg-JBNP-eGFP-Cas9mRNA delivery; and FIG. 3L shows the time-dependence of the flow cytometry analysis.

FIGS. 4A, 4B, 4C, 4D, 4E, 4F, 4G, and 4H show endosomal escape of the JBNP. FIG. 4A includes images showing the endosomal escape of Arg-JBNP and Lys-JBNP; FIG. 4B includes images showing the time-dependent endosomal escape of Lys-JBNP; FIG. 4C includes images showing inhibition of the proton sponge effect; FIG. 4D is a graph showing the results of an acid-base titration curve; FIG. 4E includes images showing endosomal escape of the JBNPs and LNPs; FIG. 4F is a graph illustrating quantification of colocalization; FIG. 4G includes images showing the antiviral activity associated with JBNP delivery; and FIG. 4H is a graph showing inhibition of gene expression of the JBNPSs and LNPs.

FIGS. 5A, 5B, 5C, 5D, 5E, 5F, 5G, and 5H show JBNP with protecting molecules. FIG. 5A shows TEM images of polyethylene glycol (PEG)-JBNT; FIGS. 5B and 5C show a UV-VIS analysis; FIG. 5D shows TEM images of PEG-JBNP-siRNA; FIG. 5E shows a UV-VIS analysis; FIG. F shows a zeta-potential analysis; FIG. G shows cell uptake of Arg-JBNP and PEG-JBNP by flow cytometry; FIG. 5H shows uptake percentage analysis of flow cytometry; and FIG. I shows MFI analysis of flow cytometry.

FIG. 6A, 6B, 6C, 6D, 6E, 6F, 6G, 6H, 6I, 6J, and 6K show multi-functional JBNTs. FIG. 6A is a graph showing the UV-Vis spectrum of ArgJBNT/GlyJBNT/AspJBNT; FIG. 6B is a graph showing the UV-Vis spectrum of calculated sum/co-assembled ArgJBNT, GlyJBNT and AspJBNT; FIG. 6C is a graph comparing the sizes of ArgJBNT/AspJBNT/GlyJBNT/co-assembled (Mix), ArgJBNT (Arg), GlyJBNT (Gly), and AspJBNT (Asp); FIG. 6D is a graph showing the zeta potentials of ArgJBNT/AspJBNT/GlyJBNT/co-assembled (mix), ArgJBNT (Arg), GlyJBNT (Gly), and AspJBNT (Asp); FIG. 6E is TEM images of ArgJBNT/AspJBNT/GlyJBNT/HisJBNT co-assembled, ArgJBNT, GlyJBNT, and AspJBNT; FIG. 6F shows the UV-Vis analysis; FIG. 6G shows the Zeta potential analysis; FIG. 6H is TEM images of co-assembled ArgJBNT, PEGLysJBNT and SPLysJBNT; FIG. 6I is TEM images of Arg-JBNP, PEG-JBNP and SP-PEG-JBNP; FIG. 6J shows flow cytometry analysis of multi-functional JBNPs; and FIG. 6K is a graph of mean fluorescence intensity obtained by flow cytometry.

FIG. 7A, Transferrin-JBNP-mRNA was delivered into cells and Cas9eGFP mRNA expressed. FIG. 7B shows peptide WYRGRL (SEQ ID NO:47) can target type II collegen in the knee joint with WYRGRL-ArgJBNP. FIG. 7C shows the quantification of signal from IVIS® Spectrum in vivo imaging.

FIG. 8A shows the results from an apoptosis assay with LysJBNP-Doxorubicin (LysJBNP-DOX). FIG. 8B shows the quantification of apoptotic cells from the results from FIG. 8A. FIG. 8C are the images showing the inhibition of spheroid formation. FIG. 8D illustrates the measured diameters of spheroids. FIG. 8E shows spheroids examined/stained for apoptosis markers. FIG. 8F is a flow-cytometry analysis examining apoptosis. FIG. 8G is a quantification of the apoptosis data of FIG. 8F. FIG. 8H has representative fluorescence images of the co-delivery of the small molecule drug and the siRNA via JBNP. FIG. 8I shows the data of an apoptosis assay examining co-delivery. FIG. 8J is the quantification data for the apoptotic assay of FIG. 9I. FIG. 8K shows microscopic images of the delivery of LysJBNP-DOX or LysJBNP-DOX-siRNA to ovarian cancer spheroids. FIG. 8L shows the time-dependent delivery of JBNP-DOX-siRNA to the spheroid. FIG. 8M shows representative images from apoptosis staining treated with control, JBNP-DOX, and JBNP-DOX-siRNA. FIG. 8N are the plots from an apoptosis assay examining control, JBNP-DOX, and JBNP-DOX-siRNA treatments. FIG. 8O is the quantification of apoptosis data of FIG. 8N.

FIG. 9A shows the biodistribution of ArgJBNP-siRNA. FIG. 9B shows the IgG immune response from ArgJBNP-siRNA as compared to Saline. FIG. 9C shows the IgM immune response of Arg-JBNP-siRNA as compared to Saline. FIG. 9D is a heatmap of the delivery efficiency of ArgJBNP-mRNA. FIG. 9E is a heatmap of the cell viability when treated with ArgJBNP-mRNA. FIG. 9F is a heatmap of the delivery (efficiency×cell viability). FIG. 9G is a graph prepared utilizing the response surface methodology (RSM) to optimize the formulation of ArgJBNP-mRNA. FIG. 9H is a surface plot of the data shown in FIG. 9F. FIG. 9I is a graph showing the uptake mechanism of ArgJBNP-mRNA. FIG. 9J shows the data from a pKa assay performed on ArgJBNP and LysJBNP. FIG. 9K shows UV-VIS analysis of Arg JBNT, RNAs, and ArgJBNP-RNA. FIG. 9L is a graph showing cell viability data for varying concentrations of ArgJBNP, lipid nanoparticles (LNP), polyethylenimine nanoparticles (PEI), and single walled carbon nanotubes (SWNT). FIG. 9M shows the data from a gel retardation assay for the references treatments. FIG. 9N illustrates an analysis of flow-cytometry shown in FIG. 9O. FIG. 9O is the flow-cytometry data for the graph of FIG. 9N. FIG. 9P shows the data from stability testing of ArgJBNP-mRNA. FIG. 9Q shows the data from UV-VIS analysis of time-dependent stability of ArgJBNp-mRNA. FIG. 9R is the Z-average size and PDI value of time-dependent stability of ArgJBNp-mRNA FIG. 9S shows zeta-potential data for time-dependent stability of ArgJBNp-mRNA. FIG. 9T shows time-dependent delivery of Cas9mRNA for ArgJBNP and Lipofectamine™ 2000 (Waltham, MA, USA). FIG. 9U shows representative time-dependent delivery of guide RNA (gRNA) via ArgJBNP or Lipofectamine™ 2000 (Waltham, MA, USA). FIG. 9V shows representative time-dependent data of gene editing with red fluorescence protein (RFP) in RFP expressing human umbilical vein endothelial cells (RFP-HUVEC) cells. FIG. 9W shows representative fluorescence images of kinetic study performed with delivering the Cas9-eGFP mRNA or gRNA-ATTO 550 to C28/I2 cells. FIG. 9X is a 3D analysis of the three articulated treatments. FIG. 9Y shows representative fluorescence images of gene editing of Ail4 chondrocytes with the articulated treatments. FIG. 9Z shows flow cytometry data examining the three treatments in Ail4 chondrocytes.

FIG. 10A is a schematic drawing of ArgJBNP-CRISPR. FIG. 10B shows the signal from IVIS® Spectrum in vivo imaging of the specified organs. FIG. 10C is the quantification of the IVIS® Spectrum in vivo imaging data from FIG. 10A. FIG. 10D fluorescence images of liver sections with 4′,6-diamidino-2-phenylindole (DAPI) staining. FIG. 10E shows representative images of live section of Ail4 mice with hematoxylin and eosin staining (H&E).

FIG. 11A shows the biodistribution of the ArgJBNP-mCherry mRNA in the articulated organs. FIG. 11B is graph showing the distribution from the IVIS® Spectrum in vivo imaging data of the articulated organs. FIG. 11C is a sodium dodecyl-sulfate polyacrylamide gel electrophoresis (SDS-PAGE) for protein corona analysis. FIG. 11D shows the data from Liquid Chromatography-tandem Mass spectrometry (LC/MS/MS) analysis of the protein corona. Additionally, FIG. 11E shows representative images of H&E stained tissue to examine immunogenicity of ArgJBNp-mRNA. FIG. 11F shows the relative change of IgG and IgM for treatment with ArgJBNp-mRNA. FIG. 11G shows the relative change for white blood cells (WBC), red blood cells (RBC), hemoglobin (HGB), and platelets (PLT) from a complete blood count (CBC) performed on injection of ArgJBNp-mRNA in BALB/cJ mice. FIG. 11H shows the percent body weight change for the four ArgJBNp-mRNA injected BALB/cJ mice.

FIG. 12A shows the UV-VIS analysis of ArgJBNp-RNAs. FIG. 12B shows the immunocytochemistry (ICC) images of Rab5 and Rab7. FIG. 12C shows the representative images of ArgJBNp-mRNA treatment with inhibitors (bafilomycin or chloroquine).

FIG. 13A shows the PEG effect on cell viability. FIG. 13B shows the biodistribution of PEG-JBNP-siRNA, as compared to JBNP-siRNA and ArgJBNP-siRNA. FIG. 13C shows quantification of the IVIS® Spectrum in vivo imaging data for ArgJBNP, LysJBNP, and PEG-JBNP in the liver.

FIG. 14A shows the combined PEG effect and cartilage targeting ability of WYRGRL-PEG-JBNP-mRNA. FIG. 14B is a representative safranin-O-stain section of a knee after receiving WYRGRL-PEG-JBNP-mRNA. FIG. 14C is a representative H&E stained section of a knee after receiving WYRGRL-PEG-JBNP-mRNA.

### DETAILED DESCRIPTION

Disclosed herein are self-assembled nanomaterials comprising Janus nanotubes which are composed of units having a single ring system. The self-assembled nanomaterials have low cytotoxicity, low immunogenicity, and demonstrate minimal side effects in vivo, and may be advantageously used to deliver a biologically active material. The self-assembled nanomaterials also demonstrate improved endosomal escape leading to high efficacy.

The self-assembled nanomaterials disclosed herein include a targeting moiety targeting specific receptors on the surface of cells to facilitate their specific and selective uptake. The co-assembly of different Janus nanotubes having different functional groups, and their use in differing amounts, allows for the design of self-assembled nanomaterials having desired properties. These properties may be adjusted based upon cellular delivery, circulation time in vivo, passive or active targeting, subcellular targeting, improved cellular uptake, and enhanced endosomal escape.

Throughout the present specification and the accompanying claims, the words “comprise,” “include,” and “have” and variations thereof such as “comprises,” “comprising,” “includes,” “including,” “has,” and “having” are to be interpreted inclusively. That is, these words are intended to convey the possible inclusion of other elements or integers not specifically recited, where the context allows. No language in the specification should be construed as indicating any non-claimed element essential to the practice of the invention.

The terms first, second, etc. as used herein are not meant to denote any particular ordering, but simply for convenience to denote a plurality of, for example, layers.

Recitation of ranges of values herein are merely intended to serve as a shorthand method of referring individually to each separate value falling within the range, unless otherwise indicated herein, each individual value is incorporated into the specification as if it were individually recited herein. Ranges may be expressed herein as from “about” (or “approximately”) one particular value, and/or to “about” (or “approximately”) another particular value. When such a range is expressed, another embodiment includes from the one particular value and/or to the other particular value. Similarly, when values are expressed as approximations, by use of the antecedent “about” or “approximately” it will be understood that the particular value forms another embodiment. It will be further understood that the endpoints of each of the ranges are disclosed both in relation to the other endpoint, and independently of the other endpoint.

All methods described herein can be performed in any suitable order unless otherwise indicated herein or otherwise clearly contradicted by context. Further, all methods described herein and having more than one step can be performed by more than one person or entity. Thus, a person or an entity can perform step (a) of a method, another person or another entity can perform step (b) of the method, and a yet another person or a yet another entity can perform step (c) of the method, etc. The use of any and all examples, or exemplary language (e.g., “such as”) provided herein is intended merely to better illuminate the invention and does not pose a limitation on the scope of the invention otherwise claimed.

Units, prefixes, and symbols are denoted in their Systeme International de Unites (SI) accepted form.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

The headings used herein are for organizational purposes only and are not meant to be used to limit the scope of the description or the claims, which can be had by reference to the specification as a whole. Accordingly, the terms defined immediately below are more fully defined by reference to the specification in its entirety.

Illustrations are for the purpose of describing a preferred embodiment of the invention and are not intended to limit the invention thereto.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system).

For example, “about” can mean within one or more standard deviations, or within +10% or 5% of the stated value.

The compositions, methods, and articles can alternatively comprise, consist of, or consist essentially of, any appropriate materials, steps, or components herein disclosed. The compositions, methods, and articles can additionally, or alternatively, be formulated so as to be devoid, or substantially free, of any materials (or species), steps, or components, that are otherwise not necessary to the achievement of the function or objectives of the compositions, methods, and articles.

All ranges disclosed herein are inclusive of the endpoints, and the endpoints are independently combinable with each other (e.g., ranges of “up to 25 wt. %, or, more specifically, 5 wt. % to 20 wt. %”, is inclusive of the endpoints and all intermediate values of the ranges of “5 wt. % to 25 wt. %,” etc.). “Combinations” is inclusive of blends, mixtures, alloys, reaction products, and the like. The terms “first,” “second,” and the like, do not denote any order, quantity, or importance, but rather are used to distinguish one element from another. The terms “a” and “an” and “the” do not denote a limitation of quantity and are to be construed to cover both the singular and the plural, unless otherwise indicated herein or clearly contradicted by context. “Or” means “and/or” unless clearly stated otherwise. Reference throughout the specification to “some embodiments”, “an embodiment”, and so forth, means that a particular element described in connection with the embodiment is included in at least one embodiment described herein, and may or may not be present in other embodiments. In addition, it is to be understood that the described elements may be combined in any suitable manner in the various embodiments. A “combination thereof” is open and includes any combination comprising at least one of the listed components or properties optionally together with a like or equivalent component or property not listed.

As used herein, the term “administering” means the actual physical introduction of a composition into or onto (as appropriate) a host or cell. Any and all methods of introducing the composition into the host or cell are contemplated according to the invention; the method is not dependent on any particular means of introduction and is not to be so construed. Means of introduction are well-known to those skilled in the art, and also are exemplified herein.

As used herein, “optional” or “optionally” means that the subsequently described event or circumstance may or may not occur, and that the description includes instances where said event or circumstance occurs and instances where it does not.

As used herein, the term “pharmaceutically acceptable” refers to compositions that are physiologically tolerable and do not typically produce an allergic or similar untoward reaction when administered to a subject, preferably a human subject. Preferably, as used herein, the term “pharmaceutically acceptable” means approved by a regulatory agency of a federal or state government or listed in the U.S. Pharmacopeia or other generally recognized pharmacopeia for use in animals, and more particularly in humans.

As used herein, the terms “treat,” “treating,” and “treatment” include inhibiting the pathological condition, disorder, or disease, e.g., arresting or reducing the development of the pathological condition, disorder, or disease or its clinical symptoms; or relieving the pathological condition, disorder, or disease, e.g., causing regression of the pathological condition, disorder, or disease or its clinical symptoms. These terms also encompass therapy and cure. Treatment means any way the symptoms of a pathological condition, disorder, or disease are ameliorated or otherwise beneficially altered. Preferably, the subject in need of such treatment is a mammal, preferably a human.

### Chemical Definitions

Compounds are described using standard nomenclature. For example, any position not substituted by any indicated group is understood to have its valency filled by a bond as indicated, or a hydrogen atom. A dash (“-”) that is not between two letters or symbols is used to indicate a point of attachment for a substituent. For example, —CHO is attached through carbon of the carbonyl group.

A pharmaceutically acceptable salt includes salts that retain the biological effectiveness and properties of the compound, and which are not biologically or otherwise undesirable. Pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group.

Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like.

The term “amino acid” refers to a molecule containing both an amino group and a carboxyl group. Exemplary amino acids include, without limitation, both the D- and L-isomers of the naturally-occurring amino acids, as well as non-naturally occurring amino acids prepared by organic synthesis or other metabolic routes. The term amino acid, as used herein, includes without limitation, α-amino acids, natural amino acids, non-natural amino acids, and amino acid analogs.

The term “α-amino acid” refers to a molecule containing both an amino group and a carboxyl group bound to a carbon which is designated the α-carbon.

The term “β-amino acid” refers to a molecule containing both an amino group and a carboxyl group in a β configuration.

The term “naturally occurring amino acid” refers to any one of the twenty amino acids commonly found in peptides synthesized in nature, and known by the one letter abbreviations A, R, N, C, D, Q, E, G, H, I, L, K, M, F, P, S, T, W, Y and V.

The following table shows a summary of the properties of natural amino acids:

| | 3- | 1- | Side- | | |
|---|---|---|---|---|---|
| | Letter | Letter | chain | Side-chain | Hydropathy |
| Amino Acid | Code | Code | Polarity | charge (pH 7.4) | Index |
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | polar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | positive (10%) | −3.2 |
| | | | | neutral (90%) | |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

“Hydrophobic amino acids” include small hydrophobic amino acids and large hydrophobic amino acids. “Small hydrophobic amino acid” are glycine, alanine, proline, and analogs thereof “Large hydrophobic amino acids” are valine, leucine, isoleucine, phenylalanine, methionine, tryptophan, and analogs thereof “Polar amino acids” are serine, threonine, asparagine, glutamine, cysteine, tyrosine, and analogs thereof “Charged amino acids” are lysine, arginine, histidine, aspartate, glutamate, and analogs thereof.

The term “amino acid analog” refers to a molecule which is structurally similar to an amino acid and that can be substituted for an amino acid in the formation of a peptidomimetic macrocycle. Amino acid analogs include, without limitation, β-amino acids, and amino acids where the amino or carboxy group is substituted by a similarly reactive group (e.g., substitution of the primary amine with a secondary or tertiary amine, or substitution of the carboxy group with an ester).

The term “non-natural amino acid” refers to an amino acid that is not one of the twenty amino acids commonly found in peptides synthesized in nature, and known by the one letter abbreviations A, R, N, C, D, Q, E, G, H, I, L, K, M, F, P, S, T, W, Y and V. Non-natural amino acids or amino acid analogs include, without limitation, structures according to the following:

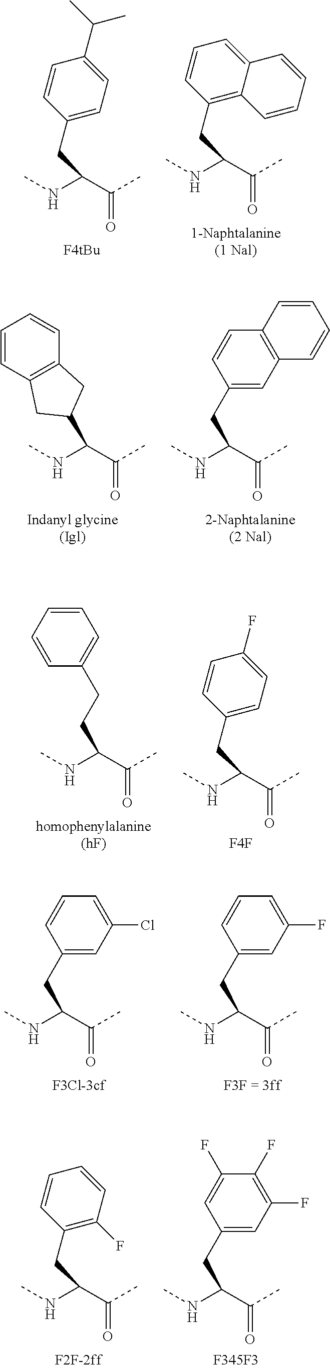

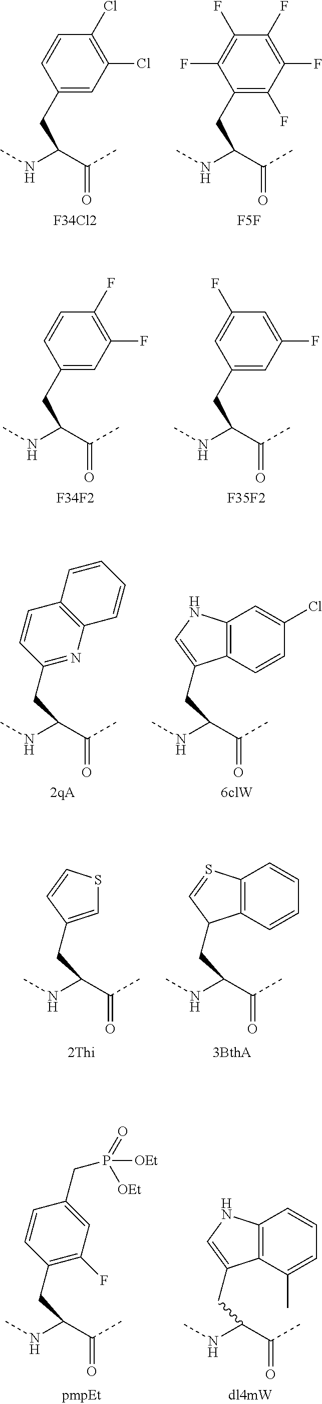

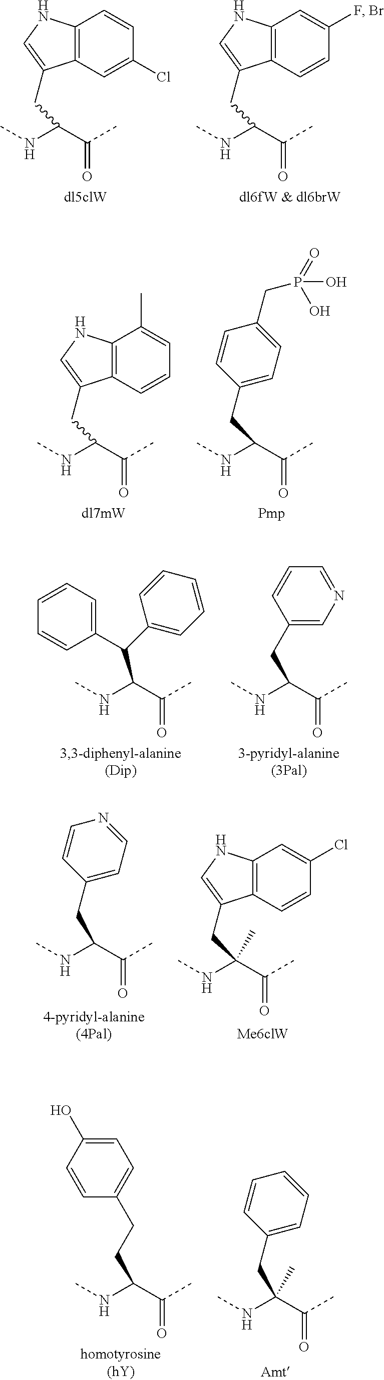

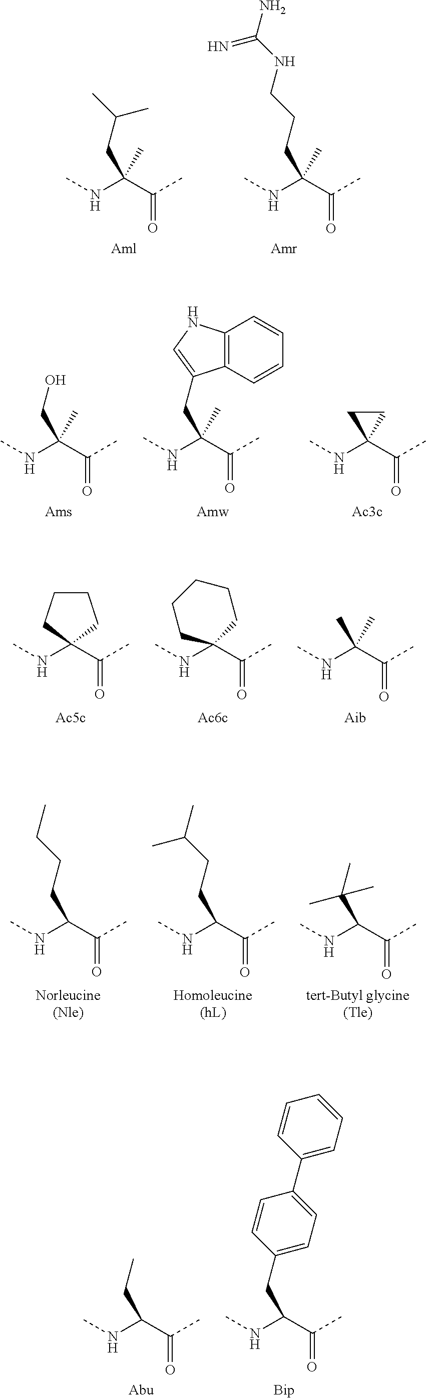

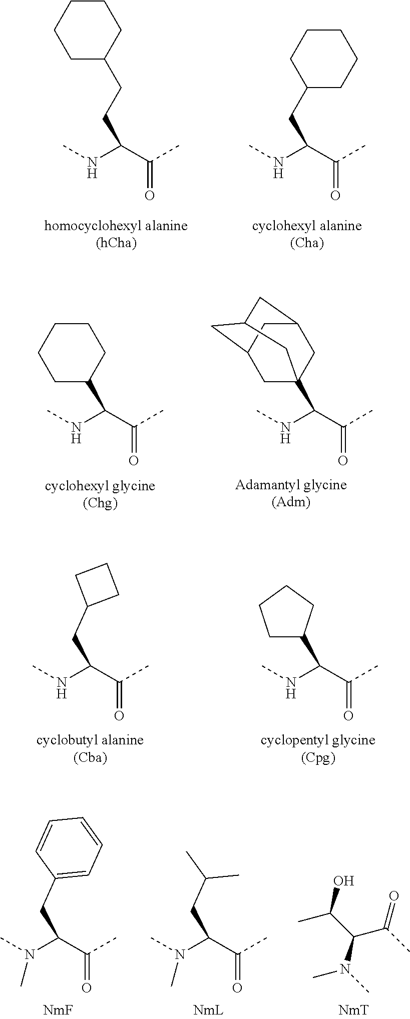

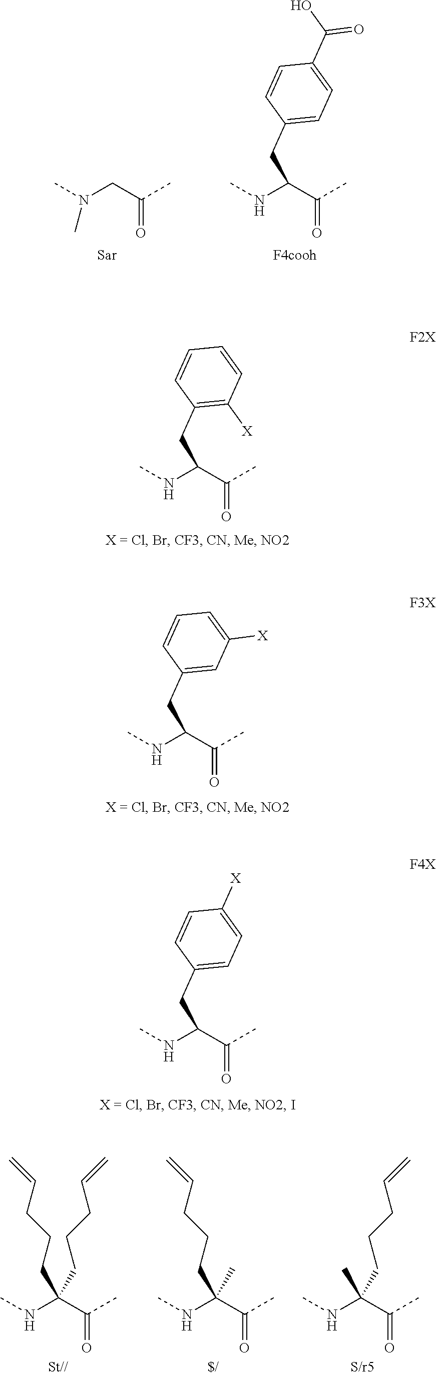

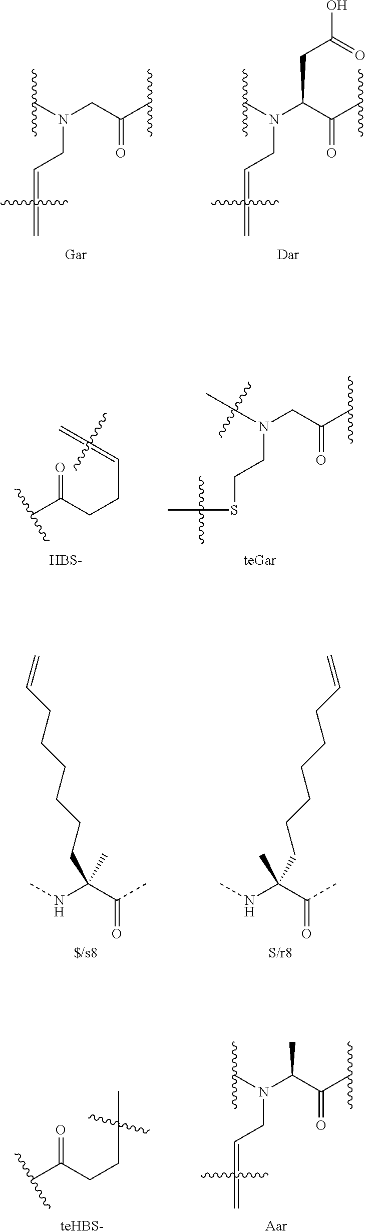

Amino acid analogs include j-amino acid analogs. Examples of β-amino acid analogs include, but are not limited to, the following: cyclic j-amino acid analogs; 0-alanine; (R)-β-phenylalanine; (R)-1,2,3,4-tetrahydro-isoquinoline-3-acetic acid; (R)-β-amino-4-(1-naphthyl)-butyric acid; (R)-β-amino-4-(2,4-dichlorophenyl)butyric acid; (R)-β-amino-4-(2-chlorophenyl)-butyric acid; (R)-β-amino-4-(2-cyanophenyl)-butyric acid; (R)-β-amino-4-(2-fluorophenyl)-butyric acid; (R)-β-amino-4-(2-furyl)-butyric acid; (R)-β-amino-4-(2-methylphenyl)-butyric acid; (R)-β-amino-4-(2-naphthyl)-butyric acid; (R)-β-amino-4-(2-thienyl)-butyric acid; (R)-β-amino-4-(2-trifluoromethylphenyl)-butyric acid; (R)-β-amino-4-(3,4-dichlorophenyl)butyric acid; (R)-β-amino-4-(3,4-difluorophenyl)butyric acid; (R)-3-amino-4-(3-benzothienyl)-butyric acid; (R)-β-amino-4-(3-chlorophenyl)-butyric acid; (R)-3-amino-4-(3-cyanophenyl)-butyric acid; (R)-β-amino-4-(3-fluorophenyl)-butyric acid; (R)-3-amino-4-(3-methylphenyl)-butyric acid; (R)-β-amino-4-(3-pyridyl)-butyric acid; (R)-3-amino-4-(3-thienyl)-butyric acid; (R)-β-amino-4-(3-trifluoromethylphenyl)-butyric acid; (R)-β-amino-4-(4-bromophenyl)-butyric acid; (R)-β-amino-4-(4-chlorophenyl)-butyric acid; (R)-β-amino-4-(4-cyanophenyl)-butyric acid; (R)-β-amino-4-(4-fluorophenyl)-butyric acid; (R)-β-amino-4-(4-iodophenyl)-butyric acid; (R)-β-amino-4-(4-methylphenyl)-butyric acid; (R)-3-amino-4-(4-nitrophenyl)-butyric acid; (R)-β-amino-4-(4-pyridyl)-butyric acid; (R)-β-amino-4-(4-trifluoromethylphenyl)-butyric acid; (R)-β-amino-4-pentafluoro-phenylbutyric acid; (R)-β-amino-5-hexenoic acid; (R)-β-amino-5-hexynoic acid; (R)-β-amino-5-phenylpentanoic acid; (R)-β-amino-6-phenyl-5-hexenoic acid; (S)-1,2,3,4-tetrahydro-isoquinoline-3-acetic acid; (S)-β-amino-4-(1-naphthyl)-butyric acid; (S)-β-amino-4-(2,4-dichlorophenyl)butyric acid; (S)-β-amino-4-(2-chlorophenyl)-butyric acid; (S)-β-amino-4-(2-cyanophenyl)-butyric acid; (S)-β-amino-4-(2-fluorophenyl)-butyric acid; (S)-β-amino-4-(2-furyl)-butyric acid; (S)-β-amino-4-(2-methylphenyl)-butyric acid; (S)-β-amino-4-(2-naphthyl)-butyric acid; (S)-3-amino-4-(2-thienyl)-butyric acid; (S)-β-amino-4-(2-trifluoromethylphenyl)-butyric acid; (S)-β-amino-4-(3,4-dichlorophenyl)butyric acid; (S)-β-amino-4-(3,4-difluorophenyl)butyric acid; (S)-β-amino-4-(3-benzothienyl)-butyric acid; (S)-β-amino-4-(3-chlorophenyl)-butyric acid; (S)-β-amino-4-(3-cyanophenyl)-butyric acid; (S)-β-amino-4-(3-fluorophenyl)-butyric acid; (S)-β-amino-4-(3-methylphenyl)-butyric acid; (S)-β-amino-4-(3-pyridyl)-butyric acid; (S)-3-amino-4-(3-thienyl)-butyric acid; (S)-β-amino-4-(3-trifluoromethylphenyl)-butyric acid; (S)-β-amino-4-(4-bromophenyl)-butyric acid; (S)-β-amino-4-(4-chlorophenyl)-butyric acid; (S)-β-amino-4-(4-cyanophenyl)-butyric acid; (S)-β-amino-4-(4-fluorophenyl)-butyric acid; (S)-3-amino-4-(4-iodophenyl)-butyric acid; (S)-β-amino-4-(4-methylphenyl)-butyric acid; (S)-3-amino-4-(4-nitrophenyl)-butyric acid; (S)-β-amino-4-(4-pyridyl)-butyric acid; (S)-β-amino-4-(4-trifluoromethylphenyl)-butyric acid; (S)-β-amino-4-pentafluoro-phenylbutyric acid; (S)-3-amino-5-hexenoic acid; (S)-β-amino-5-hexynoic acid; (S)-β-amino-5-phenylpentanoic acid; (S)-β-amino-6-phenyl-5-hexenoic acid; 1,2,5,6-tetrahydropyridine-3-carboxylic acid; 1,2,5,6-tetrahydropyridine-4-carboxylic acid; β-amino-3-(2-chlorophenyl)-propionic acid; β-amino-3-(2-thienyl)-propionic acid; β-amino-3-(3-bromophenyl)-propionic acid; β-amino-3-(4-chlorophenyl)-propionic acid; β-amino-3-(4-methoxyphenyl)-propionic acid; β-amino-4,4,4-trifluoro-butyric acid; β-aminoadipic acid; D-β-phenylalanine; β-leucine; L-β-homoalanine; L-β-homoaspartic acid γ-benzyl ester; L-β-homoglutamic acid δ-benzyl ester; L-β-homoisoleucine; L-β-homoleucine; L-β-homomethionine; L-β-homophenylalanine; L-β-homoproline; L-β-homotryptophan; L-β-homovaline; L-Nω-benzyloxycarbonyl-β-homolysine; Nω-L-β-homoarginine; O-benzyl-L-β-homohydroxyproline; O-benzyl-L-β-homoserine; O-benzyl-L-β-homothreonine; O-benzyl-L-β-homotyrosine; γ-trityl-L-β-homoasparagine; (R)-β-phenylalanine; L-β-homoaspartic acid 7-t-butyl ester; L-β-homoglutamic acid δ-t-butyl ester; L-Nω-β-homolysine; N6-trityl-L-β-homoglutamine; Nω-2,2,4,6,7-pentamethyl-dihydrobenzofuran-5-sulfonyl-L-β-homoarginine; O-t-butyl-L-β-homohydroxy-proline; O-t-butyl-L-β-homoserine; O-t-butyl-L-β-homothreonine; O-t-butyl-L-β-homotyrosine; 2-aminocyclopentane carboxylic acid; and 2-aminocyclohexane carboxylic acid.

Amino acid analogs include analogs of alanine, valine, glycine or leucine. Examples of amino acid analogs of alanine, valine, glycine, and leucine include, but are not limited to, the following: α-methoxyglycine; α-allyl-L-alanine; α-aminoisobutyric acid; α-methyl-leucine; β-(1-naphthyl)-D-alanine; β-(1-naphthyl)-L-alanine; β-(2-naphthyl)-D-alanine; β-(2-naphthyl)-L-alanine; 1-(2-pyridyl)-D-alanine; β-(2-pyridyl)-L-alanine; 3-(2-thienyl)-D-alanine; β-(2-thienyl)-L-alanine; β-(3-benzothienyl)-D-alanine; 3-(3-benzothienyl)-L-alanine; β-(3-pyridyl)-D-alanine; β-(3-pyridyl)-L-alanine; β-(4-pyridyl)-D-alanine; β-(4-pyridyl)-L-alanine; 1-chloro-L-alanine; 1-cyano-L-alanin; 3-cyclohexyl-D-alanine; 3-cyclohexyl-L-alanine; 3-cyclopenten-1-yl-alanine; 3-cyclopentyl-alanine; 3-cyclopropyl-L-Ala-OH.dicyclohexylammonium salt; β-t-butyl-D-alanine; β-t-butyl-L-alanine; 7-aminobutyric acid; L-α,β-diaminopropionic acid; 2,4-dinitro-phenylglycine; 2,5-dihydro-D-phenylglycine; 2-amino-4,4,4-trifluorobutyric acid; 2-fluoro-phenylglycine; 3-amino-4,4,4-trifluoro-butyric acid; 3-fluoro-valine; 4,4,4-trifluoro-valine; 4,5-dehydro-L-leu-OH.dicyclohexylammonium salt; 4-fluoro-D-phenylglycine; 4-fluoro-L-phenylglycine; 4-hydroxy-D-phenylglycine; 5,5,5-trifluoro-leucine; 6-aminohexanoic acid; cyclopentyl-D-Gly-OH.dicyclohexylammonium salt; cyclopentyl-Gly-OH.dicyclohexylammonium salt; D-α,β-diaminopropionic acid; D-α-aminobutyric acid; D-α-t-butylglycine; D-(2-thienyl)glycine; D-(3-thienyl)glycine; D-2-aminocaproic acid; D-2-indanylglycine; D-allylglycine.dicyclohexylammonium salt; D-cyclohexylglycine; D-norvaline; D-phenylglycine; β-aminobutyric acid; β-aminoisobutyric acid; (2-bromophenyl)glycine; (2-methoxyphenyl)glycine; (2-methylphenyl)glycine; (2-thiazoyl)glycine; (2-thienyl)glycine; 2-amino-β-(dimethylamino)-propionic acid; L-α,β-diaminopropionic acid; L-α-aminobutyric acid; L-α-t-butylglycine; L-β-thienyl)glycine; L-2-amino-β-(dimethylamino)-propionic acid; L-2-aminocaproic acid dicyclohexyl-ammonium salt; L-2-indanylglycine; L-allylglycine.dicyclohexyl ammonium salt; L-cyclohexylglycine; L-phenylglycine; L-propargylglycine; L-norvaline; N-α-aminomethyl-L-alanine; D-α,γ-diaminobutyric acid; L-α,γ-diaminobutyric acid; β-cyclopropyl-L-alanine; (N-β-(2,4-dinitrophenyl))-L-α,β-diaminopropionic acid; (N-β-1-(4,4-dimethyl-2,6-dioxocyclohex-1-ylidene)ethyl)-D-α,β-diaminopropionic acid; (N-β-1-(4,4-dimethyl-2,6-dioxocyclohex-1-ylidene)ethyl)-L-α,β-diaminopropionic acid; (N-β-4-methyltrityl)-L-α,β-diaminopropionic acid; (N-β-allyloxycarbonyl)-L-α,β-diaminopropionic acid; (N-γ-1-(4,4-dimethyl-2,6-dioxocyclohex-1-ylidene)ethyl)-D-α,T-diaminobutyric acid; (N-γ-1-(4,4-dimethyl-2,6-dioxocyclohex-1-ylidene)ethyl)-L-α,T-diaminobutyric acid; (N-7-4-methyltrityl)-D-α,γ-diaminobutyric acid; (N-7-4-methyltrityl)-L-α,γ-diaminobutyric acid; (N-7-allyloxycarbonyl)-L-α,γ-diaminobutyric acid; D-α,γ-diaminobutyric acid; 4,5-dehydro-L-leucine; cyclopentyl-D-Gly-OH; cyclopentyl-Gly-OH; D-allylglycine; D-homocyclohexylalanine; L-1-pyrenylalanine; L-2-aminocaproic acid; L-allylglycine; L-homocyclohexylalanine; and N-(2-hydroxy-4-methoxy-Bzl)-Gly-OH.

Amino acid analogs include analogs of arginine or lysine. Examples of amino acid analogs of arginine and lysine include, but are not limited to, the following: citrulline; L-2-amino-3-guanidinopropionic acid; L-2-amino-3-ureidopropionic acid; L-citrulline; Lys(Me)₂-OH; Lys(N3) OH; Nδ-benzyloxycarbonyl-L-omithine; Nω-nitro-D-arginine; Nω-nitro-L-arginine; α-methyl-omithine; 2,6-diaminoheptanedioic acid; L-omithine; (Nδ-1-(4,4-dimethyl-2,6-dioxo-cyclohex-1-ylidene)ethyl)-D-omithine; (Nδ-1-(4,4-dimethyl-2,6-dioxo-cyclohex-1-ylidene)ethyl)-L-omithine; (Nδ-4-methyltrityl)-D-omithine; (Nδ-4-methyltrityl)-L-omithine; D-omithine; L-omithine; Arg(Me)(Pbf)-OH; Arg(Me)₂-OH (asymmetrical); Arg(Me)₂-OH (symmetrical); Lys(ivDde)-OH; Lys(Me)2-OH.HCl; Lys(Me3)-OH chloride; Nω-nitro-D-arginine; and Nω-nitro-L-arginine.

Amino acid analogs include analogs of aspartic or glutamic acids. Examples of amino acid analogs of aspartic and glutamic acids include, but are not limited to, the following: α-methyl-D-aspartic acid; α-methyl-glutamic acid; α-methyl-L-aspartic acid; γ-methylene-glutamic acid; (N-γ-ethyl)-L-glutamine; [N-α-(4-aminobenzoyl)]-L-glutamic acid; 2,6-diaminopimelic acid; L-α-aminosuberic acid; D-2-aminoadipic acid; D-α-aminosuberic acid; α-aminopimelic acid; iminodiacetic acid; L-2-aminoadipic acid; threo-β-methyl-aspartic acid; γ-carboxy-D-glutamic acid γ,γ-di-t-butyl ester; γ-carboxy-L-glutamic acid γ,γ-di-t-butyl ester; Glu(OAll)-OH; L-Asu(OtBu)-OH; and pyroglutamic acid.

Amino acid analogs include analogs of cysteine and methionine. Examples of amino acid analogs of cysteine and methionine include, but are not limited to, Cys(farnesyl)-OH, Cys(farnesyl)-OMe, α-methyl-methionine, Cys(2-hydroxyethyl)-OH, Cys(3-aminopropyl)-OH, 2-amino-4-(ethylthio)butyric acid, buthionine, buthioninesulfoximine, ethionine, methionine methylsulfonium chloride, selenomethionine, cysteic acid, [2-(4-pyridyl)ethyl]-DL-penicillamine, [2-(4-pyridyl)ethyl]-L-cysteine, 4-methoxybenzyl-D-penicillamine, 4-methoxybenzyl-L-penicillamine, 4-methylbenzyl-D-penicillamine, 4-methylbenzyl-L-penicillamine, benzyl-D-cysteine, benzyl-L-cysteine, benzyl-DL-homocysteine, carbamoyl-L-cysteine, carboxyethyl-L-cysteine, carboxymethyl-L-cysteine, diphenylmethyl-L-cysteine, ethyl-L-cysteine, methyl-L-cysteine, t-butyl-D-cysteine, trityl-L-homocysteine, trityl-D-penicillamine, cystathionine, homocystine, L-homocystine, (2-aminoethyl)-L-cysteine, seleno-L-cystine, cystathionine, Cys(StBu)-OH, and acetamidomethyl-D-penicillamine.

Amino acid analogs include analogs of phenylalanine and tyrosine. Examples of amino acid analogs of phenylalanine and tyrosine include 3-methyl-phenylalanine, 3-hydroxyphenylalanine, α-methyl-3-methoxy-DL-phenylalanine, α-methyl-D-phenylalanine, α-methyl-L-phenylalanine, 1,2,3,4-tetrahydroisoquinoline-3-carboxylic acid, 2,4-dichloro-phenylalanine, 2-(trifluoromethyl)-D-phenylalanine, 2-(trifluoromethyl)-L-phenylalanine, 2-bromo-D-phenylalanine, 2-bromo-L-phenylalanine, 2-chloro-D-phenylalanine, 2-chloro-L-phenylalanine, 2-cyano-D-phenylalanine, 2-cyano-L-phenylalanine, 2-fluoro-D-phenylalanine, 2-fluoro-L-phenylalanine, 2-methyl-D-phenylalanine, 2-methyl-L-phenylalanine, 2-nitro-D-phenylalanine, 2-nitro-L-phenylalanine, 2;4;5-trihydroxy-phenylalanine, 3,4,5-trifluoro-D-phenylalanine, 3,4,5-trifluoro-L-phenylalanine, 3,4-dichloro-D-phenylalanine, 3,4-dichloro-L-phenylalanine, 3,4-difluoro-D-phenylalanine, 3,4-difluoro-L-phenylalanine, 3,4-dihydroxy-L-phenylalanine, 3,4-dimethoxy-L-phenylalanine, 3,5,3′-triiodo-L-thyronine, 3,5-diiodo-D-tyrosine, 3,5-diiodo-L-tyrosine, 3,5-diiodo-L-thyronine, 3-(trifluoromethyl)-D-phenylalanine, 3-(trifluoromethyl)-L-phenylalanine, β-amino-L-tyrosine, 3-bromo-D-phenylalanine, 3-bromo-L-phenylalanine, 3-chloro-D-phenylalanine, 3-chloro-L-phenylalanine, 3-chloro-L-tyrosine, 3-cyano-D-phenylalanine, 3-cyano-L-phenylalanine, 3-fluoro-D-phenylalanine, 3-fluoro-L-phenylalanine, 3-fluoro-tyrosine, 3-iodo-D-phenylalanine, 3-iodo-L-phenylalanine, 3-iodo-L-tyrosine, 3-methoxy-L-tyrosine, 3-methyl-D-phenylalanine, 3-methyl-L-phenylalanine, 3-nitro-D-phenylalanine, 3-nitro-L-phenylalanine, 3-nitro-L-tyrosine, 4-(trifluoromethyl)-D-phenylalanine, 4-(trifluoromethyl)-L-phenylalanine, 4-amino-D-phenylalanine, 4-amino-L-phenylalanine, 4-benzoyl-D-phenylalanine, 4-benzoyl-L-phenylalanine, 4-bis(2-chloroethyl)amino-L-phenylalanine, 4-bromo-D-phenylalanine, 4-bromo-L-phenylalanine, 4-chloro-D-phenylalanine, 4-chloro-L-phenylalanine, 4-cyano-D-phenylalanine, 4-cyano-L-phenylalanine, 4-fluoro-D-phenylalanine, 4-fluoro-L-phenylalanine, 4-iodo-D-phenylalanine, 4-iodo-L-phenylalanine, homophenylalanine, thyroxine, 3,3-diphenylalanine, thyronine, ethyl-tyrosine, and methyl-tyrosine.

Amino acid analogs include analogs of proline. Examples of amino acid analogs of proline include, but are not limited to, 3,4-dehydro-proline, 4-fluoro-proline, cis-4-hydroxy-proline, thiazolidine-2-carboxylic acid, and trans-4-fluoro-proline.

Amino acid analogs include analogs of serine and threonine. Examples of amino acid analogs of serine and threonine include, but are not limited to, 3-amino-2-hydroxy-5-methylhexanoic acid, 2-amino-3-hydroxy-4-methylpentanoic acid, 2-amino-3-ethoxybutanoic acid, 2-amino-3-methoxybutanoic acid, 4-amino-3-hydroxy-6-methylheptanoic acid, 2-amino-3-benzyloxypropionic acid, 2-amino-3-benzyloxypropionic acid, 2-amino-3-ethoxypropionic acid, 4-amino-3-hydroxybutanoic acid, and α-methylserine.

Amino acid analogs include analogs of tryptophan. Examples of amino acid analogs of tryptophan include, but are not limited to, the following: α-methyl-tryptophan; β-(3-benzothienyl)-D-alanine; β-(3-benzothienyl)-L-alanine; 1-methyl-tryptophan; 4-methyl-tryptophan; 5-benzyloxy-tryptophan; 5-bromo-tryptophan; 5-chloro-tryptophan; 5-fluoro-tryptophan; 5-hydroxy-tryptophan; 5-hydroxy-L-tryptophan; 5-methoxy-tryptophan; 5-methoxy-L-tryptophan; 5-methyl-tryptophan; 6-bromo-tryptophan; 6-chloro-D-tryptophan; 6-chloro-tryptophan; 6-fluoro-tryptophan; 6-methyl-tryptophan; 7-benzyloxy-tryptophan; 7-bromo-tryptophan; 7-methyl-tryptophan; D-1,2,3,4-tetrahydro-norharman-3-carboxylic acid; 6-methoxy-1,2,3,4-tetrahydronorharman-1-carboxylic acid; 7-azatryptophan; L-1,2,3,4-tetrahydro-norharman-3-carboxylic acid; 5-methoxy-2-methyl-tryptophan; and 6-chloro-L-tryptophan.

In some embodiments, amino acid analogs are racemic. In some embodiments, the D isomer of the amino acid analog is used. In some embodiments, the L isomer of the amino acid analog is used. In other embodiments, the amino acid analog comprises chiral centers that are in the R or S configuration. In still other embodiments, the amino group(s) of a β-amino acid analog is substituted with a protecting group, e.g., tert-butyloxycarbonyl (BOC group), 9-fluorenylmethyloxycarbonyl (FMOC), tosyl, and the like. In yet other embodiments, the carboxylic acid functional group of a β-amino acid analog is protected, e.g., as its ester derivative. In some embodiments the salt of the amino acid analog is used.

A “non-essential” amino acid residue is a residue that can be altered from the wild-type sequence of a polypeptide without abolishing or substantially abolishing its essential biological or biochemical activity (e.g., receptor binding or activation). An “essential” amino acid residue is a residue that, when altered from the wild-type sequence of the polypeptide, results in abolishing or substantially abolishing the polypeptide's essential biological or biochemical activity.

A “conservative amino acid substitution” is one in which the amino acid residue is replaced with an amino acid residue having a similar side chain. Families of amino acid residues having similar side chains have been defined in the art. These families include amino acids with basic side chains (e.g., K, R, H), acidic side chains (e.g., D, E), uncharged polar side chains (e.g., G, N, Q, S, T, Y, C), nonpolar side chains (e.g., A, V, L, I, P, F, M, W), beta-branched side chains (e.g., T, V, I) and aromatic side chains (e.g., Y, F, W, H). Thus, a predicted nonessential amino acid residue in a polypeptide, for example, is replaced with another amino acid residue from the same side chain family. Other examples of acceptable substitutions are substitutions based on isosteric considerations (e.g., norleucine for methionine) or other properties (e.g., 2-thienylalanine for phenylalanine).

The term “peptide” refers to one or more amino acid residues which are bonded together. The term “polypeptide” refers to a linear organic polymer consisting of a large number of amino-acid residues (20 or more) bonded together in a chain, forming part of (or the whole of) a protein molecule.

The term “cyclic peptide” refers to polypeptide chains which contain a circular sequence of bonds. For example, in any aspect or embodiment described herein, cyclic peptides include, without limitation, structures according to the following:

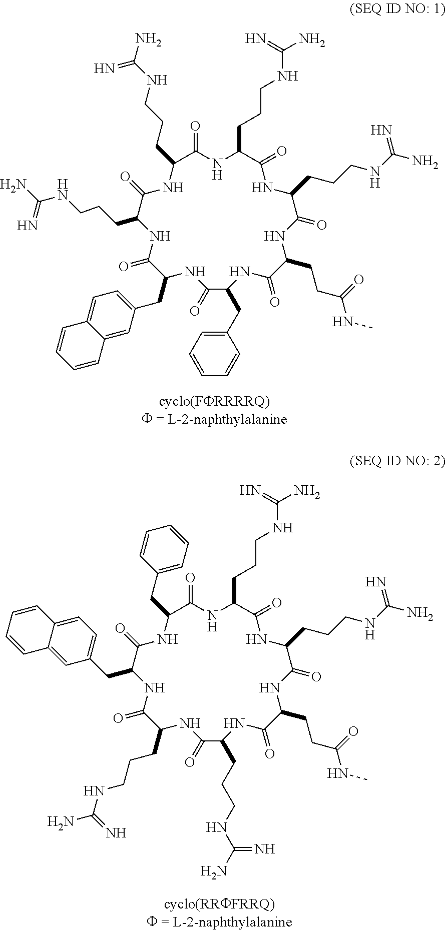

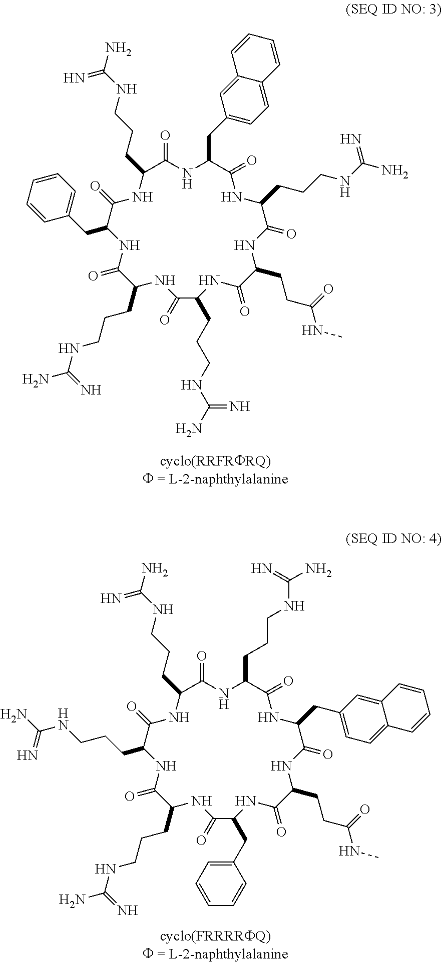

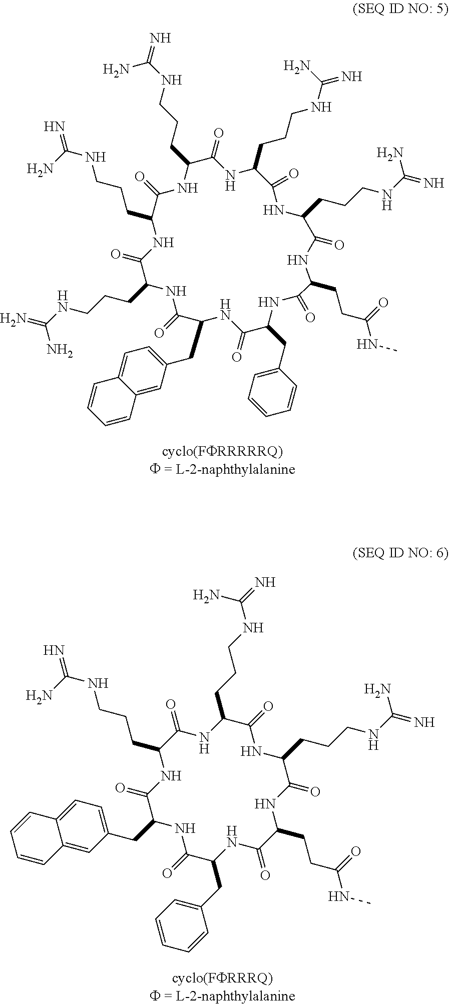

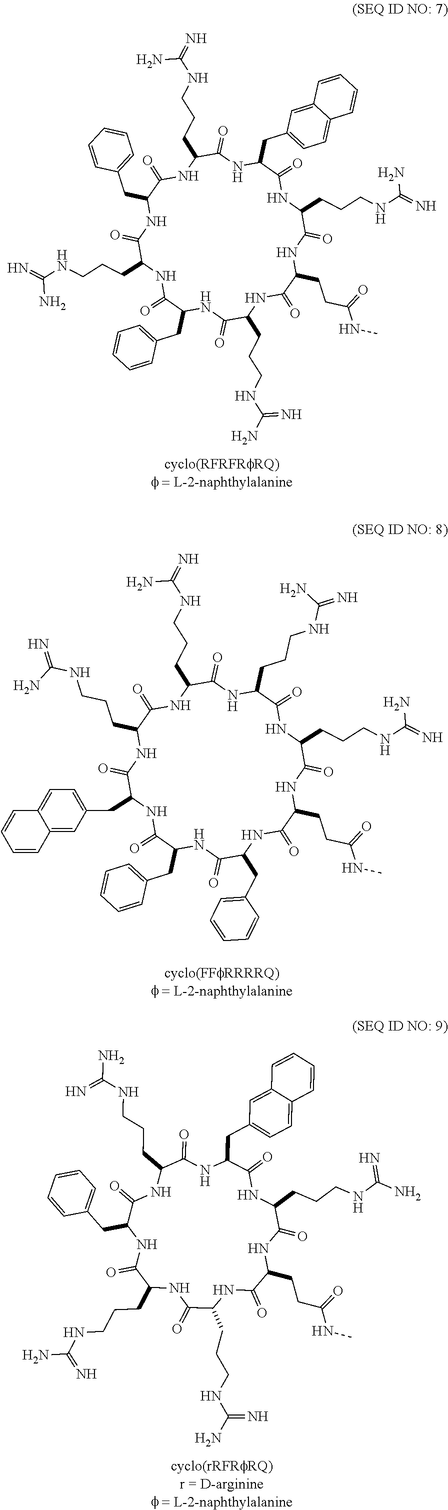

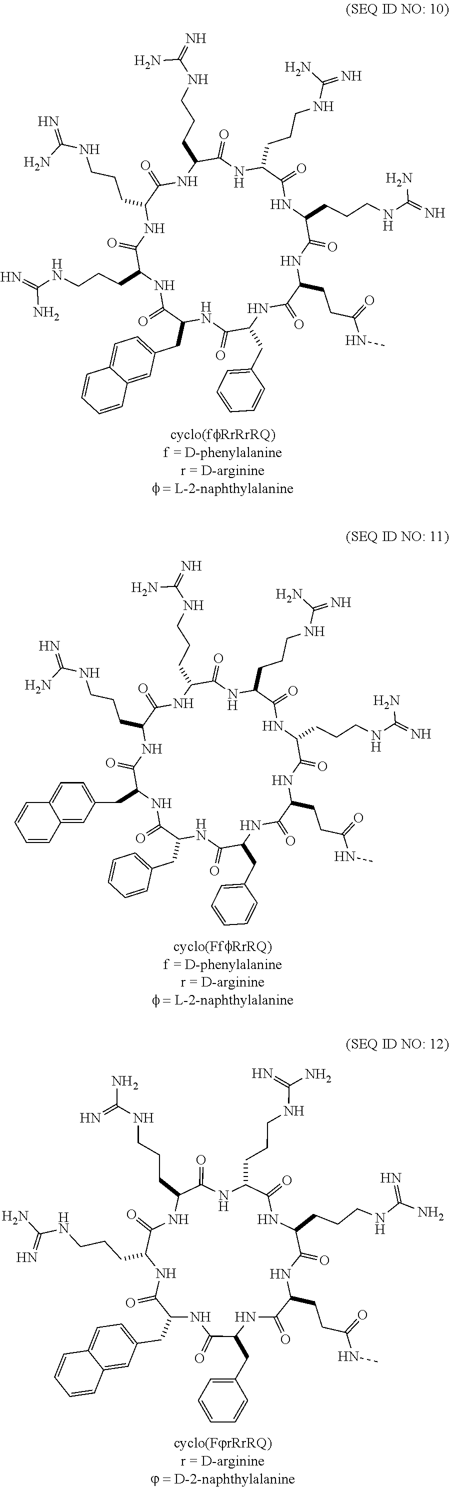

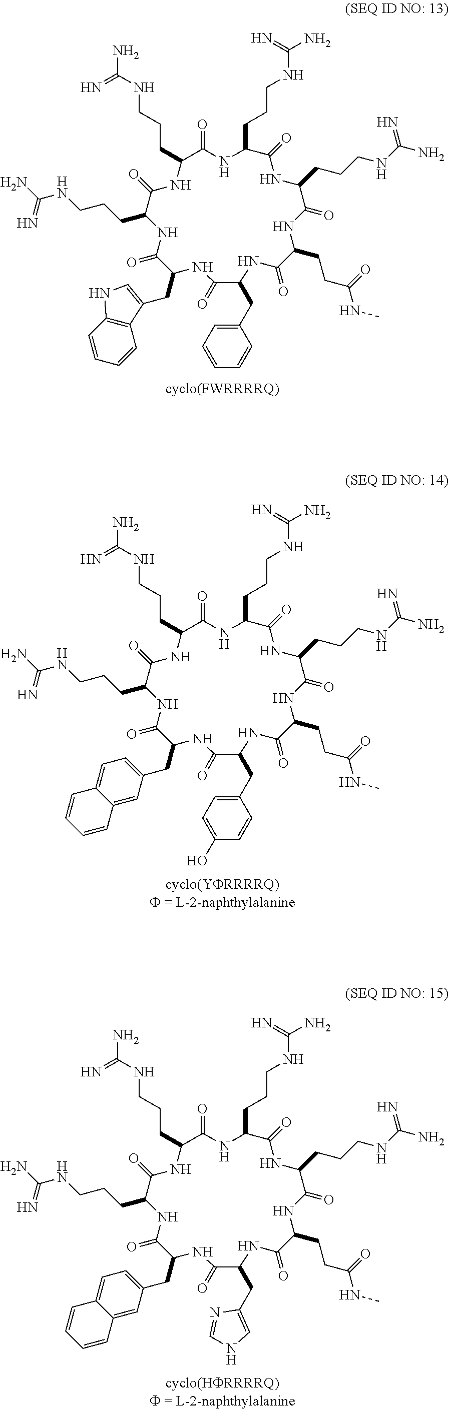

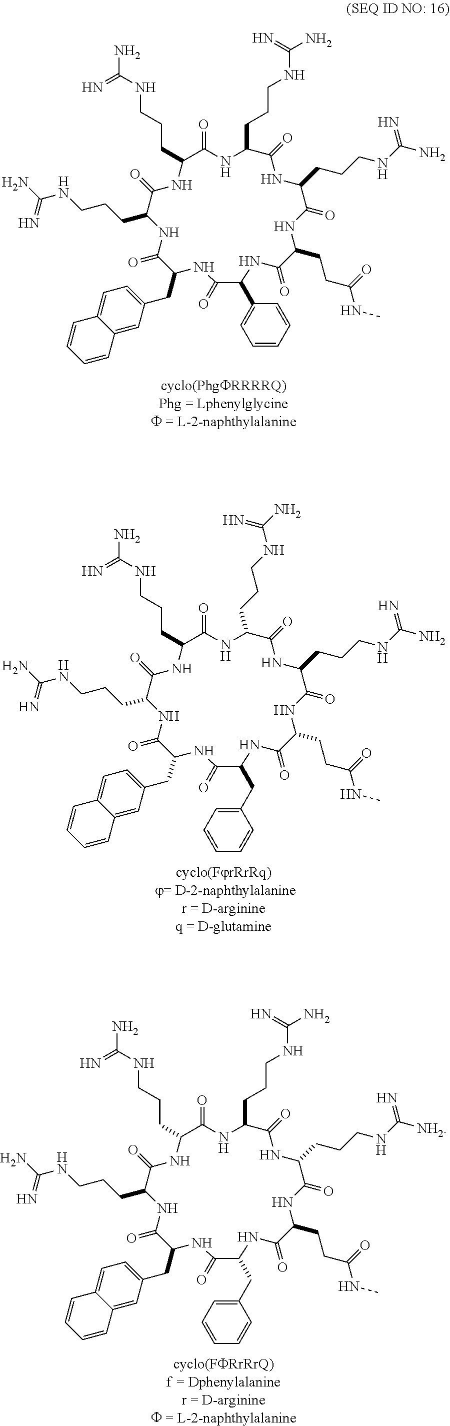

The term “α-polypeptide” refers to are polypeptides derived from α-amino acids.

The term “β-polypeptide” refers to are polypeptides derived from β-amino acids.

The term “phosphate ester” refers to esters of phosphoric acid, a central phosphate molecule with alkyl or aromatic substituents. For example, in any aspect or embodiment described herein, the phosphate esters include, without limitation, structures according to the following:

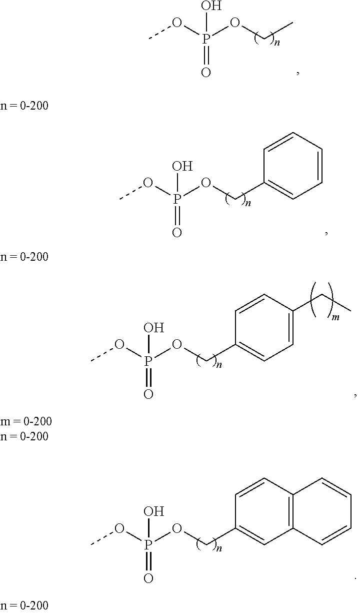

The term “aliphatic” or “aliphatic group” refers to a hydrocarbon moiety that may be straight-chain (i.e., unbranched), branched, or cyclic (including fused, bridging, and spiro-fused polycyclic) and may be completely saturated or may contain one or more units of unsaturation. Suitable aliphatic groups include, but are not limited to, linear or branched, alkyl, alkenyl, and alkynyl groups, and hybrids thereof. As used herein the terms “aliphatic” or “aliphatic group”, also encompass partially substituted analogs of these moieties where at least one of the hydrogen atoms of the aliphatic group is replaced by an atom that is not carbon or hydrogen.

The term “linker” refers to a chemical group that connects one or more other chemical groups via at least one covalent bond.

While the invention has been described with reference to an exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiments disclosed as the best mode contemplated for carrying out this invention, but that the invention will include all embodiments falling within the scope of the appended claims. Any combination of the described elements in all possible variations thereof is encompassed by the invention unless otherwise indicated herein or otherwise clearly contradicted by context.

### Self-Assembled Nanomaterial

The self-assembled nanomaterial of the present disclosure comprises a Janus base nanotube (JBNT). The JBNT is composed of structural units based on single ring system, and self-assembles into a nanomaterial which can be used for drug delivery and scaffolding. In particular, the JBNT provides a solution for the delivery of a biologically active molecule to a specific cell and/or tissue. In an embodiment the JBNT comprises a biologically active molecule covalently or non-covalently adhered to the JBNT. Further, the JBNT may be coupled/conjugated to a targeting moiety in order to facilitate active targeting of the self-assembled nanomaterial including the biologically active molecule to a specific cell and/or tissue.

The JBNT is a biocompatible, biodegradable material having relatively low cytotoxicity and low immunogenicity. The JBNT also combines the advantages of lipid nanoparticles and cationic polymers for improved endosomal escape and high efficacy. Advantageously, the JBNT efficiently enter cells via macropinocytosis (using the same mechanism as lipid nanoparticles) and can effectively escape from endosomes via the “proton sponge” effect, which is the same mechanism as cationic polymers. Therefore, the JBNT can achieve excellent delivery of a biologically active molecule and present extremely low cytotoxicity.

In an embodiment, the self-assembled nanomaterial comprising a Janus base nanotube, wherein the Janus base nanotube comprises at least one compound represented by Formulas I to XII, or a pharmaceutically acceptable salt thereof.

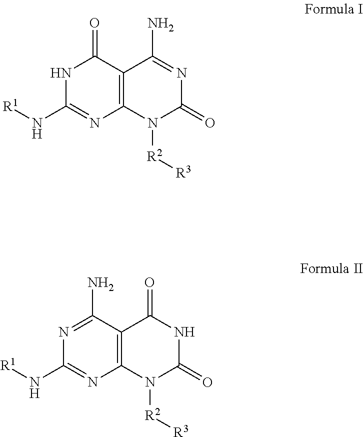

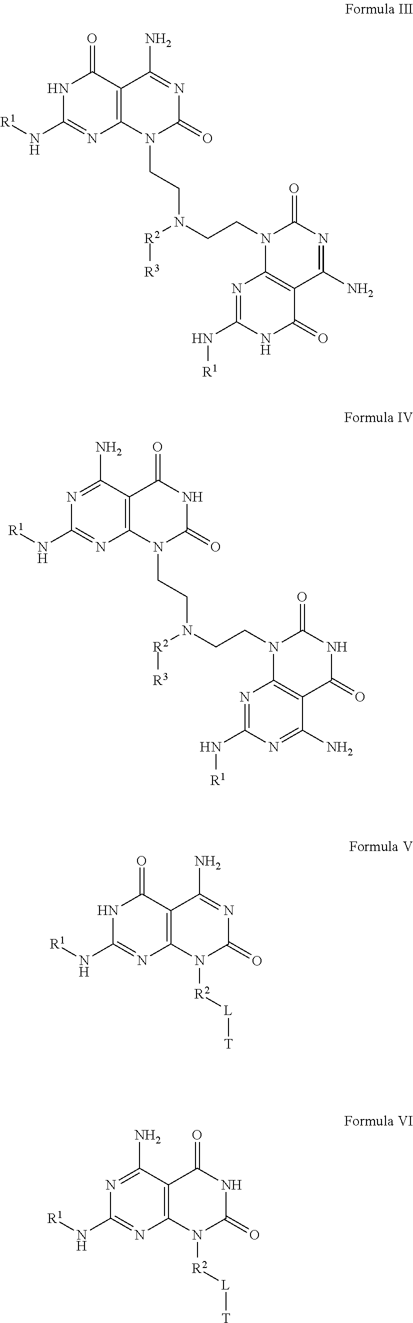

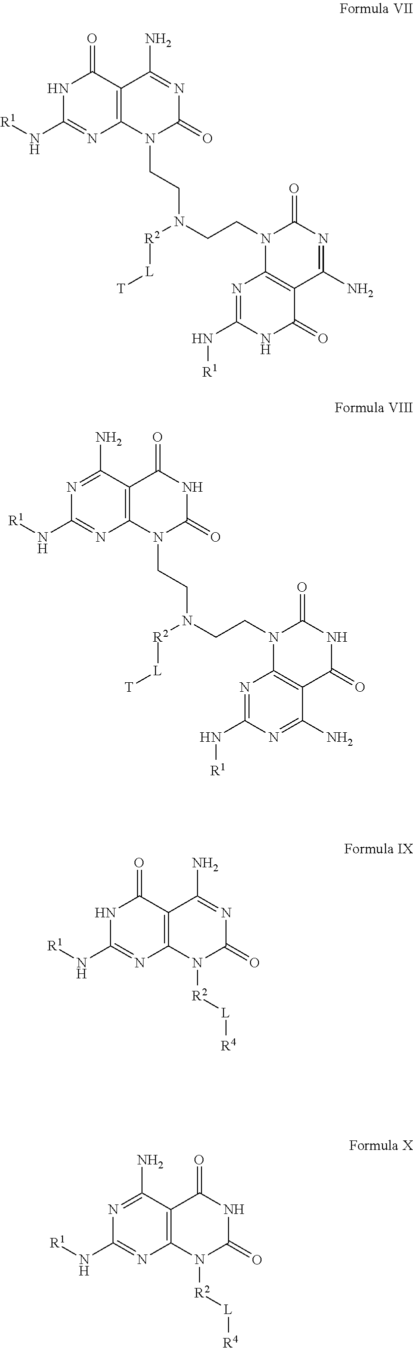

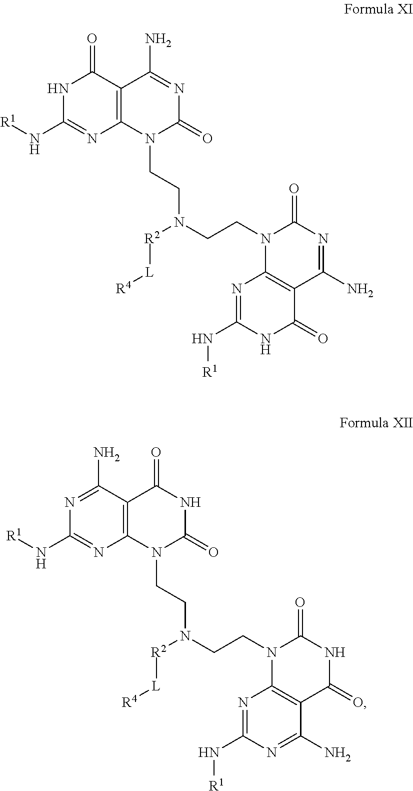

wherein, R¹ is H or CH₃; R² is (CH₂)ⱼ, (CH₂CH₂O)ₖ or (CH₂CH₂NH)ₘ, where j, k and m are independently 1-200; R³ is an α-amino acid, a β-amino acid, an α-polypeptide, or a 3-polypeptide; L is a bond or a linker group; T is a biologically active molecule or a targeting molecule; and R⁴ is a coating material. In an embodiment, T is a targeting molecule.

In any aspect or embodiment described herein, the targeting molecule or moiety includes or is a peptide, cyclic peptide, small molecule, another molecular structure mentioned herein as a targeting molecule or moiety, or a combination thereof.

In any aspect or embodiment described herein, the coating material comprises a polymer, a peptide, a polypeptide, a lipid-based material, phosphate ester, or a biomimetic membrane.

In an embodiment, L is the linker group, and is selected from an acid-cleavable group, a reducible disulfide group, an α-amino acid, a β-amino acid, an α-polypeptide, a β-polypeptide, an enzyme cleavable group, a stimuli-responsive group, or a combination thereof. The acid-cleavable group can include N-acyl hydrazone, a carbonate group, or an ester group. The reducible disulfide linker can include N-succinimidyl-4-(2-pyridyldithio)pentanoate (SPP), N-succinimidyl-4-(2-pyridyldithio)butyrate (SPDB), or 4-(4′-acetylphenoxy)butanoic acid (AcBut), Val-Cit dipeptide, Phe-Lys dipeptide, an α-methyl substituted disulfide, an engineered cysteine residue, or a thiol-containing maytansinoid. The stimuli-responsive linker can include a trans-cyclooctene linker or a thioether-containing linker. The enzyme cleavable linker can include GPLGOAGQ (SEQ ID NO:89), GDEVEAPKGC (SEQ ID NO:90), citrulline-valine, a glycosidase-cleavable linker, a (3-glucoronidase-cleavable linker, a β-Galactosidase-cleavable linker, a phosphatase-cleavable linker, a pyrophosphate-containing linker, a dipeptide-containing linker, Gly-Phe-Leu-Gly, Ala-Leu-Ala-Leu, Phe-Lys-PABC (para-aminobenzyl carbamate), a Val-Cit-PABC containing linker, a Glu-Val-Cit-containing linker, or a Val-Ala containing linker.

In an embodiment, T is a targeting molecule, and the targeting molecule is selected from a biologically active molecule, an amphiphilic polymer, an aptamer, a peptide, a protein, a polysaccharide, a polyunsaturated fatty acid, or a carbohydrate. The selection of the targeting molecule depends upon the cell and/or tissue to which the self-assembled nanomaterial is to be delivered.

In any aspect or embodiment described herein, non-limiting examples of the targeting molecule include: (i) small molecules such as folic acid, thiamine, and dimercaptosuccinic acid; (ii) proteins such as bovine serum albumin (BSA), transferrin, antibodies, nanobodies, lectins, cytokines, fibrinogen, and thrombin; (iii) polysaccharides such as hyaluronic acid, chitosan, dextran, oligosaccharides, and heparin; (iv) polyunsaturated fatty acids such as palmitic acid and phospholipids; (v) targeting molecules for infected cells/tissue targeting molecules such as RGD, c(CMGRC) (SEQ ID NO:17), PHSRN (SEQ ID NO:18), LHRD (SEQ ID NO:19), antigenic peptides, internalization peptides, cell-penetrating peptides, VP22, RPRAPARSASRPRRPVE (SEQ ID NO:20), sC18, GLRKRLRKFRNKIKEK (SEQ ID NO: 21), Peptl, and PLILLRLLRGQF (SEQ ID NO:22); (vi) targeting molecules for blood-brain barrier (BBB) penetration such as transferrin, OX26, CAQK (SEQ ID NO:23), and lactoferrin; (vii) tumor targeting molecules such as F3, KDEPQRRSARLSAKPAPPKPEPKPKKAPAKK (SEQ ID NO:24), Lyp-1, CGNKRTRGC (SEQ ID NO:25), CREKA (SEQ ID NO:26), Bld-3, CSNRDARRC (SEQ ID NO:27), AHNP (SEQ ID NO:28), YCDGFYACYMDV (SEQ ID NO:29), SP204 (KQFSALPFNFYT peptide; SEQ ID NO:30), EGF, VEGF, LFA-1, and apolipoprotein A1; (viii) targeting molecules for infarcted cardiac tissue and/or atherosclerotic-related disease such as SP204 (KQFSALPFNFYT; SEQ ID NO:30), PLGLAGGWGERDGS (SEQ ID NO:31), GGGGYDRVTIHPF (SEQ ID NO:32), VHSPNKK (SEQ ID NO:33), VHPKQHR (SEQ ID NO:34), VLTTGLPALISWIKRKRQQ (SEQ ID NO:35), NNSKSHT (SEQ ID NO:36), VHPKQHRAEEAK (SEQ ID NO:37), C*NNSKSHTC*C (SEQ ID NO:38), VHPK (SEQ ID NO:39), VHPKQHRGGSKGC (SEQ ID NO:40), Ab(429), antibody specific for VCAM-1 (e.g., Ab M/K-2.7), PECAM-1, ICAM-1 (Ab R6.5), or LFA-1 Integrin; (ix) white fat targeting molecules, such as SP204 and CKGGRAKDC (SEQ ID NO:41); (x) alveoli targeting molecules such as WGA; (xi) intestinal targeting protein such as UEA-1; (xii) membrane dipeptidase targeting molecules, such as GFE and CGFECVRQCPERC (SEQ ID NO:42); (xiii) endoplasmic reticulum (ER) targeting molecules, such as KDEL (SEQ ID NO:43) peptide, SEKDEL (SEQ ID NO:44), Eriss, and MRYMILGLLALAAVCSA (SEQ ID NO:45) peptide; (xiv) chondrocyte targeting peptides, such as RLDPTSYLRTFW (SEQ ID NO:46); (xv) cartilage targeting peptides, such as WYRGRL (SEQ ID NO:47); (xvi) mitochondrial membrane targeting molecules, such as RGD-4C-GG-D (KLAKLAK)₂ (SEQ ID NO:48), D-Arg-Dmt-Lys-Phe-NH₂, Phe-D-Arg-Phe-Lys-NH₂, D-Arg-Dmt-Orn-Phe-NH₂,D-Arg-(2′6′-dimethylTyr)-Lys-Phe-NH₂, (1,7-bis-4-hydroxy-3-methoxyphenyl-1,6-heptadiene-3,5-dione)-triphenyl-phospine, 1,5-dioctadecyl-L-glutamyl 2-histidly-hexahydrobenzoic acid-SPC-L, MSVLTPLLLRGLTGSARRLPVPRAKIHWLC (SEQ ID NO:49), GKRK (SEQ ID NO:50), and D[KLAKLAK]2 (SEQ ID NO:51); (xvii) nucleus targeting molecules, such as KKKRKV (SEQ ID NO:52), KRPAATKKAGQAKKKKL (SEQ ID NO:53), HIV-1 TAT, GRKKRRQRRRPQ (SEQ ID NO:54), R8, RRRRRRRR (SEQ ID NO:55), penetratin, RQIKIWFQNRRMKWKK (SEQ ID NO:56), HA2 peptide, GDIMGEWGNEIFGAIAAGFLG (SEQ ID NO:57), GALA (SEQ ID NO:58), WEAALAEALAEALAEHLAEALAEALEALAA (SEQ ID NO:59), Pas, FFLIPKG (SEQ ID NO:60), THRPPMWSPWVWP (SEQ ID NO:61), Angiopep2, TFFYGGSRGKRNNFKTEEY (SEQ ID NO:62), Glutathione, (γE)CG, CDX, FKESWREARGTRIERG (SEQ ID NO:63), Chlorotoxin, MCMPCFTTDHQMARKCDDCCGGKGRGKCYGPQCLCR (SEQ ID NO:64), MiniAP-4, c(DLATEPAL[Dap]) (SEQ ID NO:65), g7, GFTGFLS(Glucose) (SEQ ID NO:66), RV29, YTIWMPENPRPGTPCDIFTNSRGKRASNG (SEQ ID NO:67), iRGD, CRGDKRGPDEC (SEQ ID NO:68), IL-13p, TAMRAVDKLLLHLKKLFREGQFNRNFESIIICRDRT (SEQ ID NO:69), CGEMGWVRC (SEQ ID NO:70), Lyp-1, c(CGNKRTRGC) (SEQ ID NO:25), DOPAC-MYIEALDKYAC-COOH (SEQ ID NO:71), Pro-Lys-Lys-Lys-Arg-Lys-Val (SEQ ID NO:72), Ala-Ala-Phe-Glu-Asp-Leu-Arg-Val-Leu-Ser (SEQ ID NO:73), and Lys-Arg-Pro-Ala-Ala-Thr-Lys-Lys-Arg-Gly-Qln-Arg-Lys-Lys-Lys-Lys (SEQ ID NO:74); (xviii) MMP targeting peptides, such as GPLGIAGQ (SEQ ID NO:75); (xix) Transferrin receptor targeting peptides, such as THRPPMWSPVWP (SEQ ID NO:76); (xx) synovial targeting peptides, such as SFHQFARATLAS (SEQ ID NO:77); (xxi) tumor-associated macrophage (TAMs) targeting peptides, such as YEQDPWGVKWWY (SEQ ID NO:78), CSPGAKVRC (SEQ ID NO:79); (xxii) regulatory T lymphocytes (Tregs) targeting peptides, such as CGNKRTRGC (SEQ ID NO:25); (xxiii) myeloid-derived suppressor cells (MDSCs) targeting peptides, such as MEWSLEKGYTIK (SEQ ID NO:80).

In any aspect or embodiment described herein, T is a biologically active molecule.

In any aspect or embodiment described herein, T is a targeting molecule, and the self-assembling nanomaterial further comprises a biologically active molecule which is covalently or non-covalently adhered to the self-assembled nanomaterial. In any aspect or embodiment described herein, the self-assembled nanomaterial includes a biologically active molecule which is non-covalently adhered to (associated with) the JBNT. In any aspect or embodiment described herein, the biologically active molecule is at least partially encapsulated by the JBNT. For example, in any aspect or embodiment described herein, at least 10%, or at least 20%, or at least 30%, or at least 40%, or at least 50%, or at least 60%, or at least 80%, or 100% of the biologically active molecule is encapsulated by the JBNT.

Non-limiting examples of the biologically active agent include nucleic acids, proteins, peptides, and small molecule drugs. In an embodiment, the biologically active agent is a nucleic acid such as mRNA, guide RNA (gRNA or sgRNA), crRNA, tracrRNA, tRNA, ssDNA, dsDNA, cDNA, or a combination thereof.

In any aspect or embodiment described herein, R² is a coating material comprising a polymer, a peptide, a polypeptide, a lipid-based material, or a biomimetic membrane. The coating material may help protect the self-assembled nanomaterial from specific or non-specific clearance from the body by cells and/or organs. In any aspect or embodiment described herein, the polymer coating material includes, but not limited to, polyethylene glycol (PEG), chitosan, hyaluronic acid, a poloxamer, polyvinyl alcohol, a polysaccharide, a neutral charged poly(amino acid), negatively charged poly(amino acid), or a combination thereof. In any aspect or embodiment described herein, the peptide coating material includes, but not limited to, a “self” peptide (peptide generated by proteolytic degradation of self protein within cells expressing MHC class I or II molecules, e.g., a TCR-peptide-MHC Class II peptide), an antithrombotic peptide (e.g., CD-31 agonist peptide), CALNN (SEQ ID NO:81), CCVVT (SEQ ID NO:82), CLPFFD (SEQ ID NO:83), (γE)C(γE)C(γE)CG (SEQ ID NO:84), GCGGCGGKGGCGGCG (SEQ ID NO:85), GNYTCEVTELTREGETIIELK (SEQ ID NO:86), hexahistidine, or a combination thereof. In any aspect or embodiment described herein, the polypeptide (protein) coating material includes, but not limited to, phytochelatin, GCK15, PD-L1, CD47, CD24, beta-2-microglobulin, bovine serum albumin (BSA), hydrophobin, clusterin/ApoJ, fibrinogen, or a combination thereof. In any aspect or embodiment described herein, the lipid-based coating material includes, but not limited to, natural waxes (e.g., carnauba wax, candelilla wax, rice bran wax, beeswax), petroleum based waxes (e.g., paraffin and polyethylene wax), petroleum-based oil, mineral oil, vegetable oil, acetoglycerides, fatty acids, resins (e.g., shellac and wood rosin), or a combination thereof. In any aspect or embodiment described herein, the biomimetic membrane coating material includes, but not limited to, the membranes of red blood cells (RBC), white blood cells (WBC), cancer cells, mesenchymal stem cells, platelets, beta cells, or a combination thereof. A combination comprising at least one (e.g., 1, 2, 3, 4, 5, 6, 7, or more) of the foregoing coating materials may also be used.

In any aspect or embodiment described herein, the self-assembled nanomaterial can include a single type of JBNT or can include more than one JBNT. In any aspect or embodiment described herein, the self-assembled nanomaterial is co-assembled from more than one (e.g., a plurality) JBNT, each having properties different from one another. For example, in any aspect or embodiment described herein, the different types of JBNT can have properties that increase the hydrophobicity, stability, and/or self-assembly of the self-assembled nanomaterial. The properties may be designed to affect the cellular delivery, including proton sponge effect, circulation time in vivo, passive or active targeting, subcellular targeting, improved cellular uptake, and/or enhanced endosomal escape of the self-assembled nanomaterial.

In any aspect or embodiment described herein, the Janus base nanotube comprises at least one compound represented by Formulas I to IV.

In any aspect or embodiment described herein, the Janus base nanotube comprises at least one compound represented by Formulas V to VIII.

In any aspect or embodiment described herein, the Janus base nanotube comprises at least one compound represented by Formulas IX to XIII.

In any aspect or embodiment described herein, the Janus base nanotube comprises a combination of:
a compound represented by Formulas I to IV and a compound represented by Formulas V to VIII;
a compound represented by Formulas I to IV and a compound represented by Formulas IX to XIII;
a compound represented by Formulas V to VIII and a compound represented by Formulas IX to XIII;
a compound represented by Formulas I to IV, a compound represented by Formulas V to VIII, and a compound represented by Formulas IX to XIII; or
a combination thereof.

In any aspect or embodiment described herein, the total amount of JBNT present in the self-assembled nanomaterial ranges from 0.1 weight % (wt %) to 99.9 wt %, or from 1 wt % to 90 wt %, based on the total weight of the self-assembled nanomaterial. In any aspect or embodiment described herein, the total concentration of the JBNT in the self-assembled nanomaterial ranges from 1 microgram per milliliter (μg/mL) to 1 gram per milliliter (g/mL).

In any aspect or embodiment described herein, the self-assembled nanomaterial is in the form of fibrils, having an average diameter of 5 nm to 500 nm, or 5 nm to 250 nm, or 10 nm to 100 nm, or 20 nm to 60 nm, and an average length of 75 nm to 100 mm, or 100 nm to 10 mm, or 100 nm to 5 mm, or 100 nm to 1 mm, or 125 nm to 500 μm.

In any aspect or embodiment described herein, the pH of the self-assembled nanomaterial is from 1 to 10.

In any aspect or embodiment described herein, the self-assembled nanomaterial can have a single or multiple compartment structure. As used herein, in any aspect or embodiment described herein, a single compartment nanomaterial includes a single population of self-assembled nanomaterials, that is, a single type of JBNT and one or more ECM molecules adhering to the JBNT. As used herein, in any aspect or embodiment described herein, a multiple compartment nanomaterial includes two or more populations of self-assembled nanomaterials that form a multi-compartmental structure, through electrostatic layer-by-layer assembly. For example, in any aspect or embodiment described herein, opposite electrostatic charges on the first and second populations of JBNTs can drive assembly of the multiple compartment nanomaterial. By assembling the first and second populations in order, one population will form the interior compartment and the second population will form the exterior compartment.

In any aspect or embodiment described herein, the self-assembled nanomaterial can optionally be combined with an extracellular matrix (ECM) molecule. In any aspect or embodiment described herein, the self-assembled nanomaterials described herein are tunable materials comprising the Janus base nanotube and the ECM. In any aspect or embodiment described herein, the JBNT can assemble with different ECMs to form different nanomatrix materials, e.g., scaffold, for different cells/tissues. In any aspect or embodiment described herein. these nanomatrix materials can have a single or multiple compartment structure. In any aspect or embodiment described herein, the nanomatrix materials can be fabricated with multi-functional layers or compartments to achieve various functions (e.g., supporting cell growth, drug release). In any aspect or embodiment described herein, the ECM molecules include, but not limited to, hydroxyapatite, fibronectin, Matn1, MAtn3, laminin, a collagen (e.g., type I collagen, type II collagen), elastin, vitronectin, fibrillin, perlecan, fibrinogen, osteonectin, tenascin, thrombospondin, an intercellular adhesion molecule (ICAM1-5), an integrin, a proteoglycan (aggrecan, a glycosaminoglycan (e.g., hyaluronic acid, chondroitin sulfate, dermatin sulfate, keratan sulfate, heparin, heparin sulfate), a glycoprotein, or a combination thereof.

In any aspect or embodiment described herein, the weight ratio of JBNTs to ECM molecule is 1000:1 to 1:1.

In any aspect or embodiment described herein, the coating material includes polyethylene glycol (PEG). In any aspect or embodiment described herein, the coating material includes PEG and the self-assemble nanomaterials includes a targeting molecule.

In any aspect or embodiment described herein, the JBNP or JBNT comprises a targeting molecule comprising the amino acid sequence WYRGRL and miR140 (WYRGRL-JBNP-miR140). In any aspect or embodiment described herein, the WYRGRL-JBNP-miR140 is used and effective in the treatment, prevent, or amelioration of one or more symptoms of osteoarthritis.

In any aspect or embodiment described herein, the JBNP or JBNT comprises CRISPR targeting sgIL-1R (JBNP-CRISPR-sgIL-1R). In any aspect or embodiment described herein, the JBNP-CRISPR-sgIL-1R is used in the treatment, prevent, or amelioration of one or more symptoms of osteoarthritis, such as inflammation (e.g., inflammation in the knee joint).

In any aspect or embodiment described herein, the JBNP or JBNT comprises IL-1R siRNA (JBNP-IL-1R siRNA). In any aspect or embodiment described herein, the JBNP-IL-1R siRNA is used and effective in the treatment, prevent, or amelioration of one or more symptoms of osteoarthritis, such as inflammation (e.g., inflammation in the knee joint).

In any aspect or embodiment described herein, the JBNP or JBNT comprises IL-1RA mRNA (JBNP-IL-1RA mRNA). In any aspect or embodiment described herein, the JBNP-IL-1RA mRNA is used and effective in the treatment, prevent, or amelioration of one or more symptoms of osteoarthritis, such as inflammation (e.g., inflammation in the knee joint). In any aspect or embodiment described herein, the mRNA sequence of an IL-1RA is:

(SEQ ID NO: 87)
ATGGAAATCTGCAGAGGCCTCCGCAGTCACCTAATCACTCTCCTCCTCT
TCCTGTTCCATTCAGAGACGATCTGCCGACCCTCTGGGAGAAAATCCAG
CAAGATGCAAGCCTTCAGAATCTGGGATGTTAACCAGAAGACCTTCTAT
CTGAGGAACAACCAACTAGTTGCTGGATACTTGCAAGGACCAAATGTCA
ATTTAGAAGAAAAGATAGATGTGGTACCCATTGAGCCTCATGCTCTGTT
CTTGGGAATCCATGGAGGGAAGATGTGCCTGTCCTGTGTCAAGTCTGGT
GATGAGACCAGACTCCAGCTGGAGGCAGTTAACATCACTGACCTGAGCG
AGAACAGAAAGCAGGACAAGCGCTTCGCCTTCATCCGCTCAGACAGTGG
CCCCACCACCAGTTTTGAGTCTGCCGCCTGCCCCGGTTGGTTCCTCTGC
ACAGCGATGGAAGCTGACCAGCCCGTCAGCCTCACCAATATGCCTGACG
AAGGCGTCATGGTCACCAAATTCTACTTCCAGGAGGACGAGTAG.

In any aspect or embodiment described herein, the JBNP or JBNT comprises IL-1RA peptide mRNA (JBNP-IL-1RA peptide mRNA). In any aspect or embodiment described herein, the JBNP-IL-1RA peptide mRNA is used and effective in the treatment, prevent, or amelioration of one or more symptoms of osteoarthritis, such as inflammation (e.g., inflammation in the knee joint). In any aspect or embodiment described herein, the mRNA sequence of an IL-1RA peptide is:

(SEQ ID NO: 88)
ATGCGGCCTAGCGGCAGAAAGTCTAGCAAGATGCAGGCCTTTAGAATCT
GGGACGTTAATCAGAAAACCTTCTACCTCAGAAACAACCAGCTGGTCGCC
GGCTACCTGCAGGGCCCCAACGTGAATCTGGAAGAGAAGATCGACGTGG
TGCCAATCGAGCCTCACGCCCTGTTTCTGGGCATCCACGGCGGAAAAAT
GTGCCTGTCTTGTGTGAAGTCCGGAGATGAGACAAGACTGCAACTGGAA
GCCGTGAACATTACAGACCTGAGCGAGAACCGGAAGCAGGACAAGCGGT
TCGCCTTCATCAGAAGCGACAGCGGCCCTACAACCAGCTTCGAGAGCGC
CGCTTGCCCCGGCTGGTTCCTGTGCACCGCCATGGAAGCTGATCAGCCT
GTGTCCCTGACCAACATGCCTGATGAAGGCGTGATGGTGACCAAGTTCT
ACTTCCAGGAGGACGAGATGTGA.

In any aspect or embodiment described herein, the JBNT or JBNP includes a lysine or arginine side chain. In any aspect or embodiment described herein, the JBNT or JBNP includes a lysine or arginine side chain to target the self-assembled nanomaterials to the liver.

In any aspect or embodiment described herein, the JBNT or JBNP includes a histine side chain. In any aspect or embodiment described herein, the JBNT or JBNP includes a histidine side chain to target the self-assembled nanomaterials to organs other than the liver.

In any aspect or embodiment described herein, the JBNT or JBNP comprises a cartilage targeting molecule, such as WYRGRL or RLDPTSYLRTFW. In any aspect or embodiment described herein, the JBNT or JBNP comprises a cartilage targeting molecule, such as WYRGRL or RLDPTSYLRTFW, is used in a method to deliver the JBNT or JBNP to cartilage.

In any aspect or embodiment described herein, the JBNP comprises an agent that is mRNA, optionally with a targeting molecule, and optionally a reporter or label (e.g., tdTomato mRNA, Cy5, AF488, etc.). In any aspect or embodiment described herein, the JBNP comprises an agent that is mRNA (JBNP-mRNA), optionally with a targeting molecule, and optionally a reporter/label (.g., tdTomato mRNA, Cy5, AF488, etc) that is delivered to the liver, kidney, brain, lung, spleen, lymph nodes, bone, muslbe, heart, pancreas, intestine, solid tumor, or a combination thereof.

In any aspect or embodiment described herein, the JBNP comprises Cas9mRNA and sgRB1. In any aspect or embodiment described herein, the JBNP comprising Cas9mRNA and sgRB1 is used in a method to deliver the sgRB1 to (and edit) liver cells.

In any aspect or embodiment described herein, the JBNP comprises CRISPR (e.g., Cas9mRNA and gRNA) and a tumor targeting moiety. In any aspect or embodiment described herein, the JBNP comprising CRISPR (e.g., Cas9mRNA and gRNA) and a tumor targeting moiety is used in a method of delivering and editing the DNA of a tumor cell.

In any aspect or embodiment described herein, the JBNT and JBNP due not cause acute toxicity, an innate immune response, an adaptive immune response, or a combination thereof, when administered.

In any aspect or embodiment described herein, the side chain of the JBNT is modified to facilitate a particular type of cellular update. For example, in any aspect or embodiment described herein, lysine-based JBNTs are used to facilitate cellular uptake of JBNPs through micropinocytosis. By way of further example, in any aspect or embodiment described herein, arginine-based JBNTs are used to facilitate cellular update of JBNPs through clathrin-mediated endocytosis.

In any aspect or embodiment described herein, the JBNP comprises lysine-based JBNTs, which have significantly better endosomal escape that appears to be due to the proton sponge effect

In any aspect or embodiment described herein, the JBNP comprises arginine-based JBNTs, which have significantly enhanced endosomal escape that appears to be due to pore-forming effect to escape early endosome.

In any aspect or embodiment described herein, the JBNP of the present disclosure have lower cell-cytotoxicity than lipid nanoparticles, polymer nanoparticles (such as poly-l-lysine (PLL) nanoparticles or polyethylenimine (PEI) nanoparticles), single-wall nanotubes, or a combination thereof.

In any aspect or embodiment described herein, the JBNP comprises Cas9mRNA-eGFP and gRNA-ATTO550.

### Pharmaceutical Compositions

The self-assembled nanomaterials disclosed herein can be formulated as an injectable composition. In any aspect or embodiment described herein, the injectable compositions comprise the self-assembled nanomaterials and a pharmaceutically acceptable carrier. In any aspect or embodiment described herein, the self-assembled nanomaterials may be administered parenterally in a sterile medium, either subcutaneously, intravenously, intramuscularly, intrasternally, or by infusion techniques, in the form of sterile injectable aqueous or oleaginous suspensions. Advantageously, in any aspect or embodiment described herein, an adjuvant(s), such as a local anesthetic, preservative and buffering agents, can be dissolved in the vehicle.

The pH of pharmaceutical composition including the self-assembled nanomaterial may be at a physiological pH.

This disclosure is further illustrated by the following examples, which are non-limiting.

This disclosure is further illustrated by the following examples, which are non-limiting.

### EXAMPLES

Materials: All reagents and solvents disclosed herein were obtained from commercial suppliers and used without further purification. Commercial suppliers include Sigma-Aldrich, Alfa Aesar, Fisher Scientific and Thermo Fisher.

### Example 1

The compound designated ArgJBNT was prepared as follows.

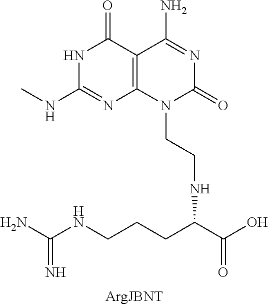 [SMILES: CNc1nc2c(c(N)nc(=O)n2CCN[C@@H](CCCNC(=N)N)C(=O)O)c(=O)[nH]1]

Synthesis of compound A3. Previously synthesized compounds A1 (96 mg) and A2 (154 mg) were dissolved in 1,2-dichloroethane (DCE, 4 mL). N,N-diisopropylethylamine (DIPEA, 78 μL) was added to the reaction mixture and after stirring at room temperature for 15 min, solid NaBH(OAc)₃ (49 mg) was added to the solution. The resulting slurry was then stirred at room temperature for 24 hours. After completion, the reaction mixture was quenched with water, and extracted with dichloromethane. The organic layers were combined and washed with brine. After drying over MgSO₄, filtration, and evaporation of solvent under reduced pressure, the crude product A3 (156 mg) was obtained and used in the next step without further purification.

Synthesis of compound ArgJBNT. Compound A3 (156 mg) was added into 94% TFA/thioanisole (2.8 mL) solution. After stirring at room temperature for 72 hours, diethyl ether (Et₂O) was added. A white precipitate formed and was then centrifuged down. After pouring out the supernatant, the white precipitate was then washed with Et₂O to yield a crude product. The crude product was purified using HPLC to produce compound ArgJBNT (37 mg, 70%). 1H NMR (500 MHz, DCl/D2O) δ 4.62-4.43 (m, 2H), 4.11 (dd, J=7.4, 4.8 Hz, 1H), 3.58-3.45 (m, 2H), 3.26-3.19 (m, 2H), 3.03 (s, 3H), 2.07-1.94 (m, 2H), 1.80-1.60 (m, 2H). HRMS (ESI) [M+H]+ calculated for 409.2055, found 409.2078.

Reaction scheme 1 illustrates the formation of compound ArgJBNT.

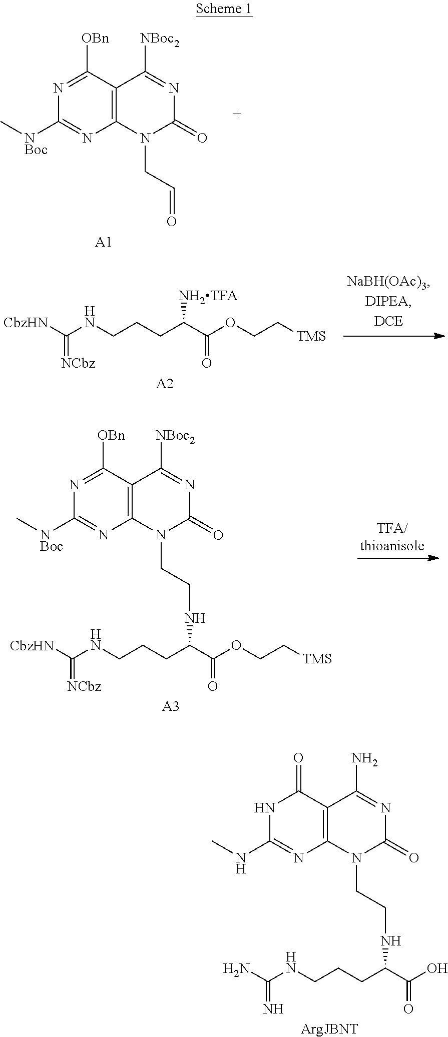

### Example 2

The compound designated GlyJBNT was prepared as follows.

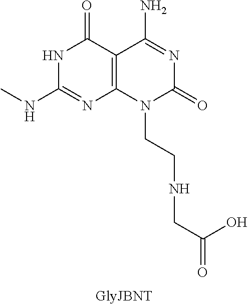 [SMILES: CNc1nc2c(c(N)nc(=O)n2CCNCC(=O)O)c(=O)[nH]1]

Synthesis of compound A5. Previously synthesized compound A1 (96 mg) and commercially available compound A4 (40 mg) were dissolved in 1,2-dichloroethane (DCE, 4 mL). N,N-diisopropylethylamine (DIPEA, 78 μL) was added to the reaction mixture and after stirring at room temperature for 15 min, solid NaBH(OAc)₃ (49 mg) was added to the solution. The resulting slurry was then stirred at room temperature for 24 hours. After completion, the reaction mixture was quenched with water, and extracted with dichloromethane. The organic layers were combined and washed with brine. After drying over MgSO₄, filtration, and evaporation of solvent under reduced pressure, the crude product A5 (102 mg) was used in the next step without further purification.

Synthesis of compound GlyJBNT. Compound A5 (102 mg) was added into 94% TFA/thioanisole (2.8 mL) solution. After stirring at room temperature for 72 hours, diethyl ether (Et₂O) was added. A white precipitate formed and then centrifuged down. After pouring out the supernatant, the white precipitate was washed with Et₂O to yield crude product. The crude product was purified using HPLC to produce compound GlyJBNT (34 mg, 85%). ¹H NMR (500 MHz, DMSO-d6) δ 11.83 (br, m, 1H), 8.88 (br, m, 2H), 8.54 (br, m, 1H), 8.13 (br, m, 1H), 7.70 (br, m, 1H), 4.34 (br, m, 2H), 3.88 (br, m, 2H), 3.26 (br, m, 2H), 2.93 (br, m, 3H). HRMS (ESI) [M+H]⁺ calculated for 310.2158, found 310.1208.

Reaction scheme 2 illustrates the formation of compound GlyJBNT.

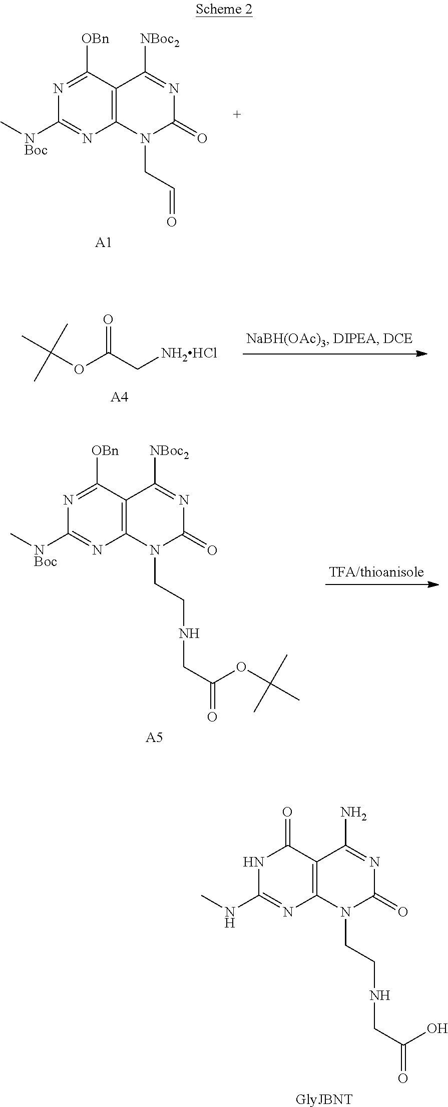

### Example 3

The compound designated AspJBNT was prepared as follows.

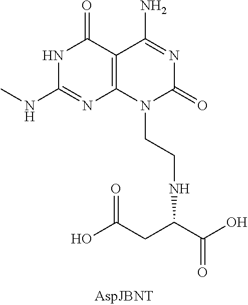 [SMILES: CNc1nc2c(c(N)nc(=O)n2CCN[C@@H](CC(=O)O)C(=O)O)c(=O)[nH]1]

Synthesis of compound A7. Previously synthesized compound A1 (96 mg) and commercially available compound A6 (68 mg) were dissolved in 1,2-dichloroethane (DCE, 4 mL). N,N-diisopropylethylamine (DIPEA, 78 μL) was added to the reaction mixture and after stirring at room temperature for 15 min, solid NaBH(OAc)₃ (49 mg) was added to the solution. The resulting slurry was then stirred at room temperature for 24 hours. After completion, the reaction mixture was quenched with water, and extracted with dichloromethane. The organic layers were combined and washed with brine. After drying over MgSO₄, filtration, and evaporation of solvent under reduced pressure, the crude product A7 (110 mg) was used in the next step without further purification.

Synthesis of compound AspJBNT. Compound A7 (110 mg) was added into 94% TFA/thioanisole (2.8 mL) solution. After stirring at room temperature for 72 hours, diethyl ether (Et₂O) was added. A white precipitate formed and was then centrifuged down. After pouring out the supernatant, the white precipitate was washed with Et₂O to yield crude product. The crude product was purified using HPLC to produce compound AspJBNT (47 mg, 98%). ¹H NMR (500 MHz, DMSO-d6) δ 11.77 (br, m, 1H), 8.51 (br, m, 1H), 8.06 (br, m, 1H), 7.55 (br, m, 1H), 4.34 (br, m, 2H), 4.16 (br, m, 1H), 2.92 (br, m, 3H), 2.85 (br, m, 2H). HRMS (ESI) [M+H]⁺ calculated for 368.1313, found 368.1278.

Reaction scheme 3 illustrates the formation of compound AspJBNT.

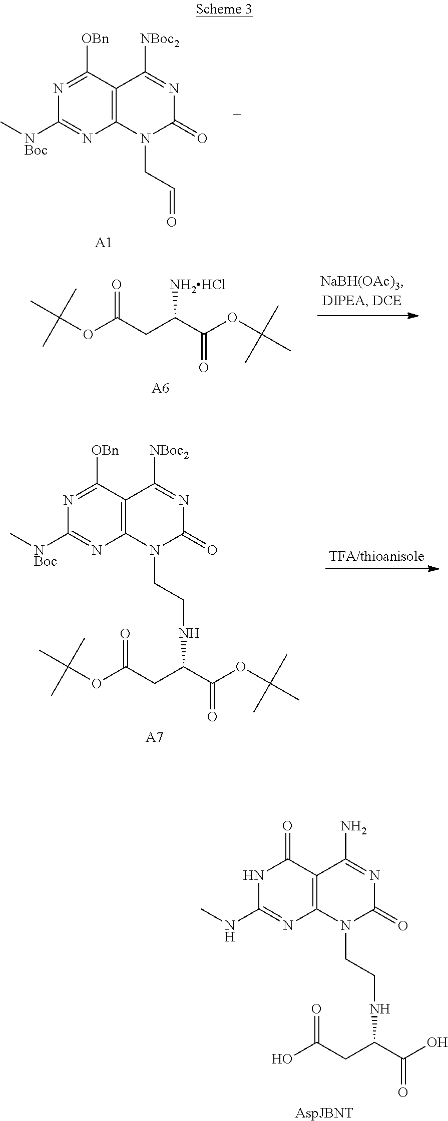

### Example 4

The compound designated HisJBNT was prepared as follows.

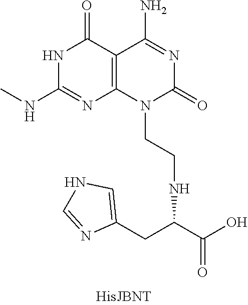 [SMILES: CNc1nc2c(c(N)nc(=O)n2CCN[C@@H](Cc2c[nH]cn2)C(=O)O)c(=O)[nH]1]

Synthesis of compound A9. Previously synthesized compound A1 (96 mg) and commercially available compound A8 (56 mg) were dissolved in 1,2-dichloroethane (DCE, 4 mL). N,N-diisopropylethylamine (DIPEA, 100 μL) was added to the reaction mixture and after stirring at room temperature for 15 min, solid NaBH(OAc)₃ (49 mg) was added to the solution. The resulting slurry was then stirred at room temperature for 24 hours. After completion, the reaction mixture was quenched with water and extracted with dichloromethane. The organic layers were combined and washed with brine. After drying over MgSO₄, filtration, and evaporation of solvent under reduced pressure, the crude product A9 (17 mg) was used in the next step without further purification.

Synthesis of compound HisJBNT. Compound A7 (17 mg) was added into 94% TFA/thioanisole (0.44 mL) solution. After stirring at room temperature for 72 hours, diethyl ether (Et₂O) was added. A white precipitate formed and was then centrifuged down. After pouring supernatant out, the white precipitate was then washed with Et₂O to yield crude product. The crude product was purified using HPLC to produce compound HisJBNT (8 mg, 14%). ¹H NMR (500 MHz, DMSO-d6) δ 11.77 (br, m, 1H), 8.51 (br, m, 1H), 8.06 (br, m, 1H), 7.88 (br, m, 1H), 7.55 (br, m, 1H), 7.14 (br, m, 1H), 4.34 (br, m, 2H), 4.16 (br, m, 1H), 2.92 (br, m, 3H), 2.85 (br, m, 2H). HRMS (ESI) [M+H]⁺ calculated for 390.1633, found 390.1601.

Reaction scheme 4 illustrates the formation of compound HisJBNT.

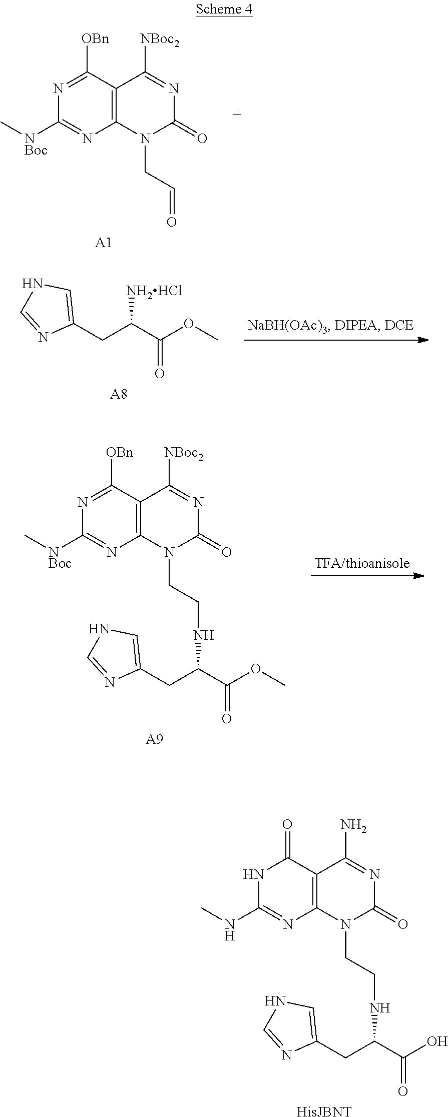

### Example 5

The compound designated PEGLysJBNT was prepared as follows.

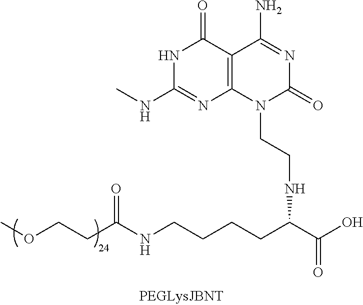

Synthesis of compound PEGLysJBNT. A solution of synthesized LysJBNT (1 mg) in aq. NaHCO₃ (0.8 mL, 0.01 M) was added to a solution of MS(PEG)24 Methyl-PEG-NHS-Ester (5 mg) in DMF (0.2 mL). After 24 hours, the reaction mixture was purified using HPLC to produce compound PEGLysJBNT (1 mg, 33%). HRMS (ESI) [M+H]⁺ calculated for 1479.8390, found 1479.8654.

Reaction scheme 5 illustrates the formation of PEGLysJBNT.

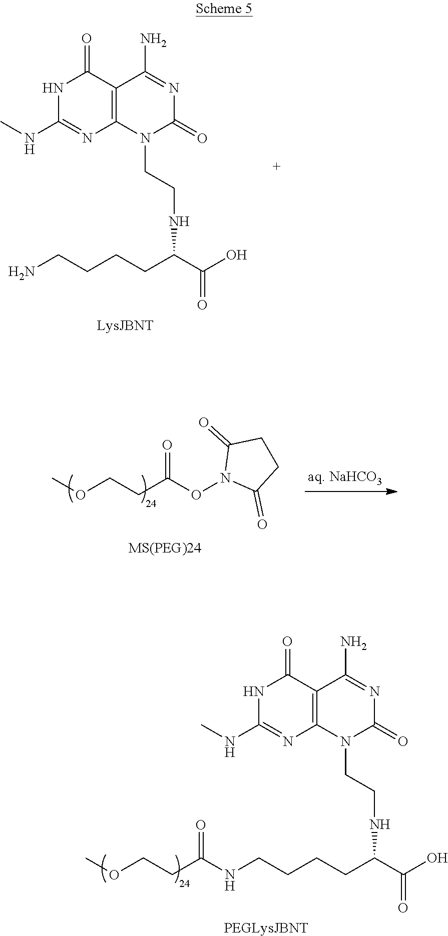

### Example 6

The compound designated LPLysJBNT, was prepared as follows.

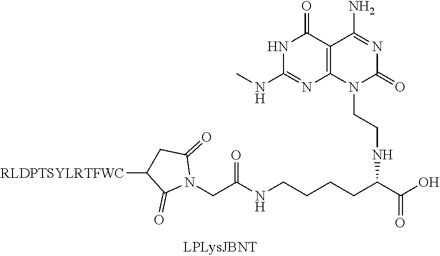

Synthesis of compound LPLysJBNT. A solution of previous synthesized LysJBNT (1 mg) in aqueous NaHCO₃ (0.8 mL, 0.01 M) was added to a solution of N-α-maleimidomethyl succinimide ester (AMAS, 1 mg) in DMF (0.2 mL). After 24 hours, peptide RLDPTSYLRTFWC (7 mg) which targets human collagen was added to the reaction mixture and reacted for another 24 hours. The reaction mixture was purified using HPLC to produce compound LPLysJBNT (5 mg, 100%). HRMS (ESI) [M+2H]²⁺ calculated for 1095.5226, found 1095.5038.

Reaction scheme 5 illustrates the formation of LPLysJBNT.

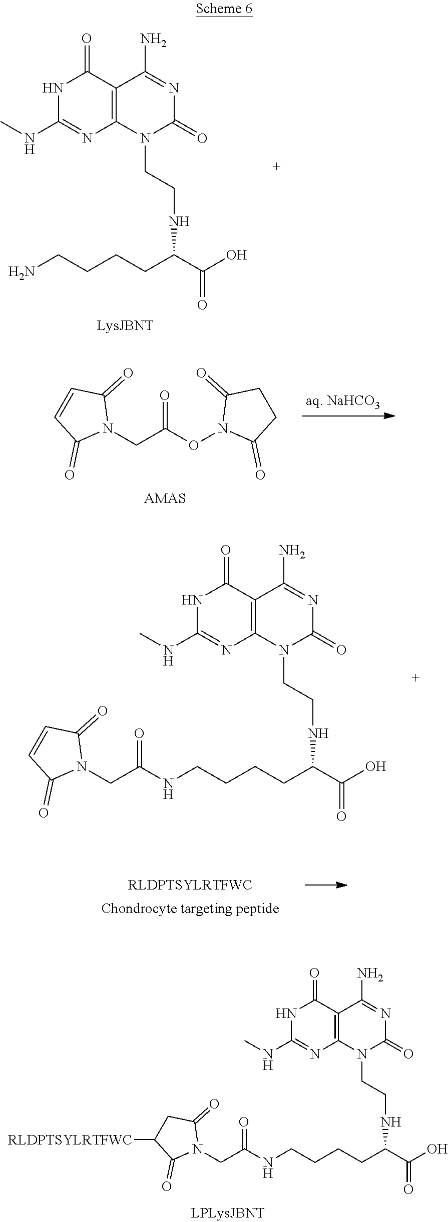

### Example 7

The compound designated SPLysJBNT was prepared as follows.

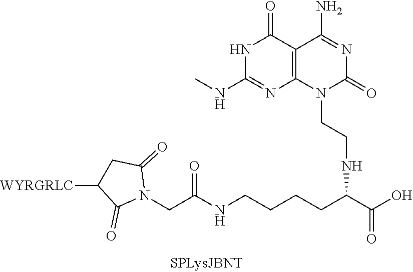

Synthesis of compound SPLysJBNT. A solution of previous synthesized LysJBNT (1 mg) in aq. NaHCO₃ (0.8 mL, 0.01 M) was added to a solution of N-α-maleimidomethyl succinimide ester (AMAS, 1 mg) in DMF (0.2 mL). After 24 hours, the targeting peptide WYRGRLC (4 mg) which targets human collagen was added to the reaction mixture and waited for another 24 hours. The reaction mixture was purified using HPLC to produce compound SPLysJBNT (3 mg, 100%). HRMS (ESI) [M+3H]³⁺ calculated for 490.8984, found 490.8820.

Reaction scheme 5 illustrates the formation of SPLysJBNT.

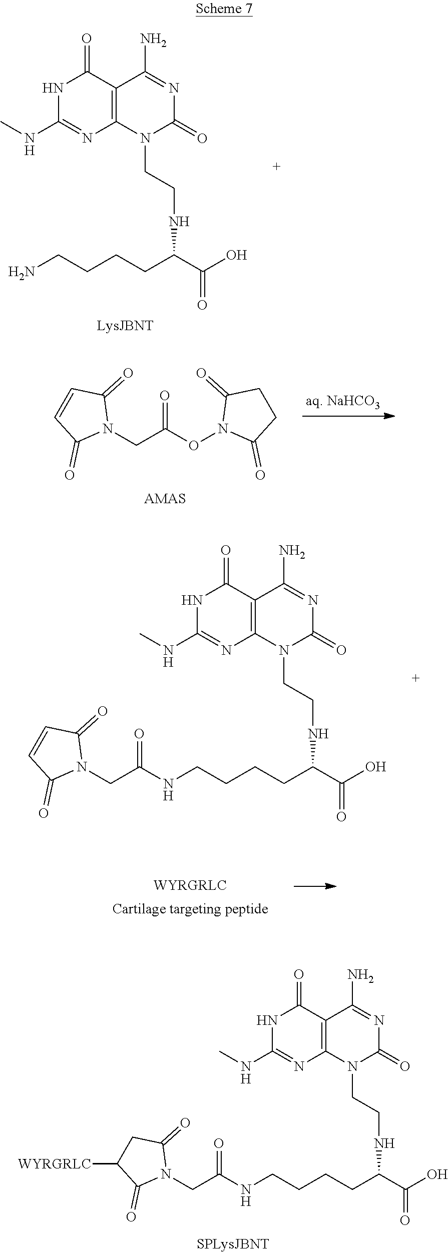

### Example 8: Multi-Functional Janus Based Nanoparticle (JBNP)

Multi-functional JBNTs. Multi-functional JBNTs were combined at an appropriate molar ratio at room temperature for 24 h to facilitate self-assembly of the JBNT. To prepare JBNTs composed of PEGLysJBNT and ArgJBNT, the molar ratio of PEGLys/ArgJBNT is 0.05:1. To prepare JBNTs composed of Peptide-LysJBNT, PEG LysJBNT and ArgJBNT, the molar ratio of Peptide-LysJBNT/PEGLys/ArgJBNT is 0.05:0.05:1.

Multi-functional JBNPs. Multi-functional JBNPs were prepared by mixing a cargo molecule (e.g., RNA, biologically active small molecule, protein) with the multi-functional JBNT at an appropriate molar ratio in nuclease-free water, followed by sonication with a sonicator at 100% amplitude, for 2 min and 30 s, at room temperature. The concentrations of different components are shown as below: c(JBNT)=1 mg/mL; c(siRNA)=5 uM; c(mRNA)=0.05 mg/mL. c(protein)=0.1 mg/mL. The ratios (volume to volume ratio) for each cargo: (4:1 ratio (Arg-JBNT:siRNA), (20:1 ratio Arg-JBNT:mRNA), (3:1 Arg-JBNT: protein).

Arg-JBNP-siRNA were assembled by mixing Arg-JBNT(1 mg/ml) and siRNA-AF488 (5 uM) in nuclease-free water as ratio of volume to volume of 4:1. Arg-JBNP-mRNA were assembled by mixing Arg-JBNT (1 mg/ml) to Cas9mRNA (0.05 mg/ml) as ratio of volume to volume of 20:1. Arg-JBNP-albumin were assembled by mixing Arg-JBNT (1 mg/ml) to albumin (0.1 mg/ml) as ratio of volume to volume, 3:1. Then mixture of Arg-JBNT and cargoes in nuclease-free water are sonicated with sonicator at 100% amplitude for 2 min and 30 sec.

JBNP Characterization. The particles and ζ potential of the JBNPs were measured by dynamic light scattering (Zetasizer) and the morphology was observed by transmission electron microscope (TEM). The get retardation assay was conducted at 0.8% low-melting agarose gel followed by electrophoresis. The UV-Vis absorption spectra were recorded with a NanoDrop One. The buffering capacity of JBNPs and polymers, JBNP and cationic polymers at the same 0.08 μmol were titrated by either adding the 2 μL of 10 mM HCl or 10 mM NaOH.

A Nikon A1 confocal laser scanning microscope was used for fluorescence imaging.

### Example 9: Characterization and Testing

Lys-JBNP delivery. Assembled Lys-JBNPs (30 μg/ml) were transfected with C28/I2 cells and then incubated at 37° C. and 5% CO₂ for 24 h or 48 h. Then, the cells were fixed with 4% formaldehyde, treated with Triton™ X-100, and stained with rhodamine phalloidin (30 min) and DAPI (10 min). Uptake of the siRNA-AlexaFluor® 488 (AllStars Neg. siRNA488, Qiagen), was quantified by flow cytometry after 24 h or 48 h following transfection into the cells. For the siRNA knockdown study, assembled Lys-JBNPs (30 μg/ml) were used to deliver GAPDH siRNA (On-TARGETplus Human GAPDH siRNA, Horizon Discovery) for 24 h. Lipofectamine™ 2000 (Invitrogen) was used as a control according to the manufacturer's protocol. The gene expression of GAPDH was analyzed by RT-PCR.

Endosomal escape study. For endosomal escape studies, Lysotracker™ Red was added to the cells prior to fixing with 4% formaldehyde. The degree of colocalization of Lysotracker™ Red and JBNPs was quantified based upon Pearson's correlation coefficient (R) using Image J software following the colocalization threshold and coloc2 plugin.

Cell uptake mechanisms study. C28/I2 human chondrocyte cells were exposed to several different inhibitors for 1 h, specifically, with chlorpromazine (Cpz) hydrochloride (100 μM for 30 min), methyl-β-cyclodextrin (Mβcd, 1 mM for 30 min), cytochrome D (CytD, 4 μM for 1 h), latrunculin-A (2 μM for 30 min), bafilomycin A1 (2001 nM for 30 min), and chloroquine (10 μM for 30 min).

Antiviral study. GFP expressing, RGD fiber modified adenovirus was added to human lung fibroblast cells. JBNPs or LNP containing the eGFP siRNA were contacted with the infected cells for 24 h. A fluorescence microscope was used to obtain cell images.

### Results for Examples 1-9

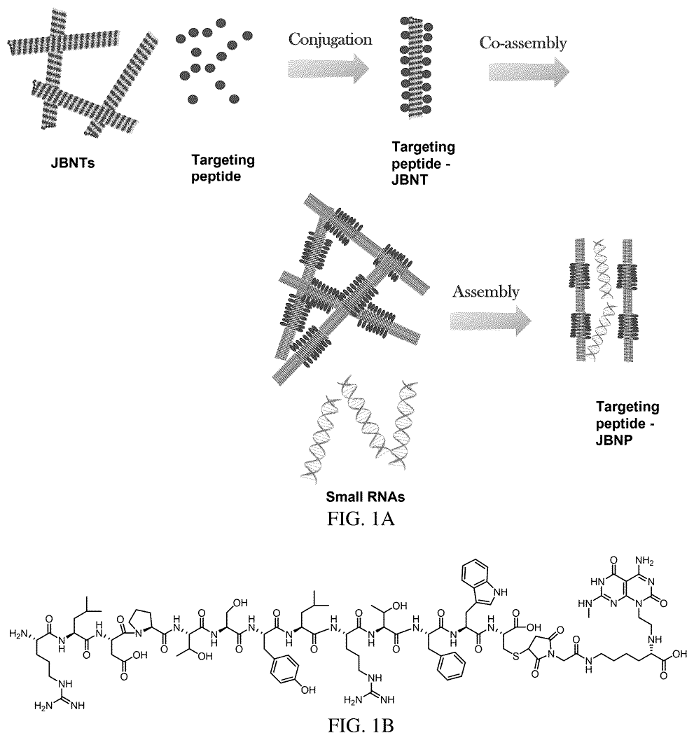

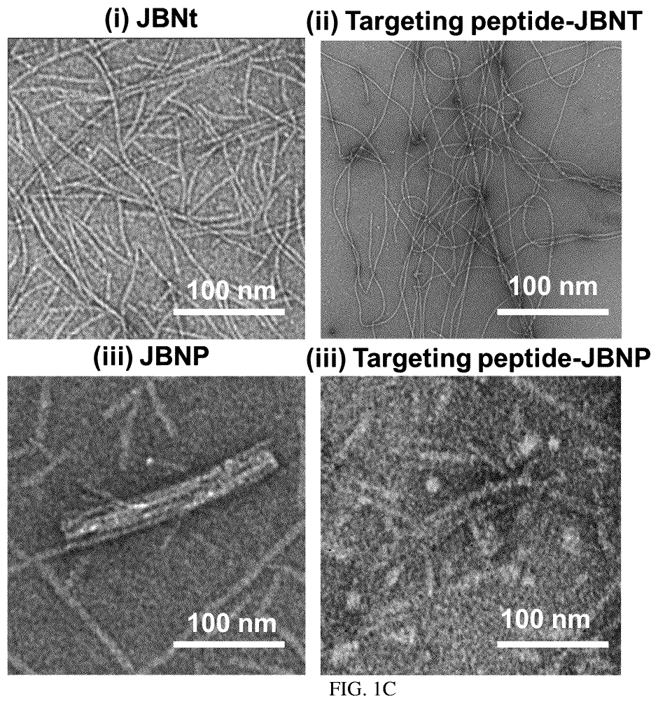

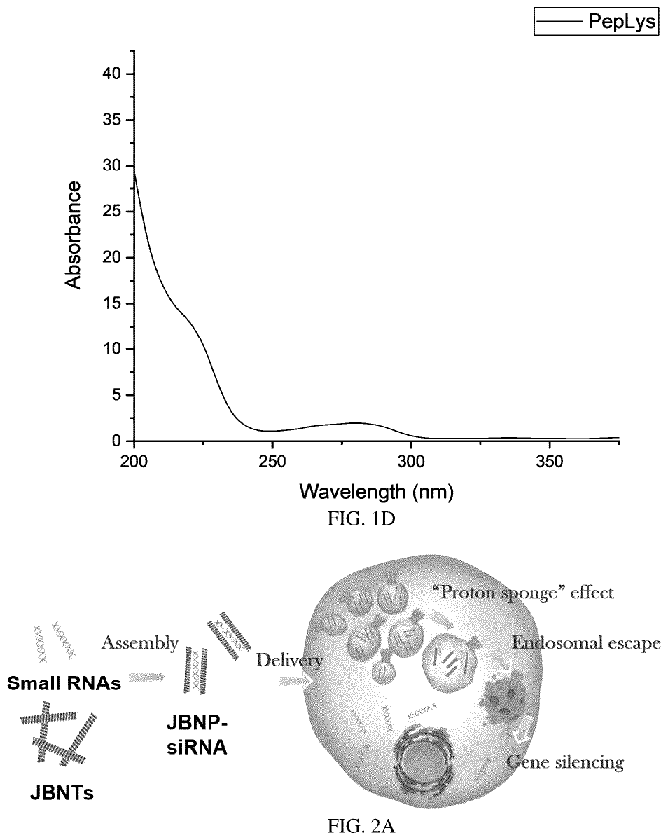

JBNT successfully delivers RNA with enhanced endosomal escape and high biocompatibility. As illustrated in FIG. 1A, the targeting moiety (peptide) was conjugated to the JBNTs and co-assembled with the JBNTs to provide targeting peptide-JBNT. In particular, the JBNTs are combined with the targeting peptide under conditions which facilitate binding of the peptide to the JBNT. In general, the binding of the targeting peptide is covalent. The JBNT containing the targeting peptide is contacted with a biologically active molecule (e.g., a nucleic acid (e.g., siRNA), protein, and/or a small molecule drug). The structure of an exemplary targeting peptide-JBNT is shown in FIG. 1B.

By optimizing the molar ratios of JBNT, targeting peptide and biologically active molecule, it was possible to make the targeting peptide-JBNP shown in FIG. 1C (i) LysJBNT ii) RLDPTSYLRTFWC peptide-PEGLysJBNT iii) LysJBNP iv) RLDPTSYLRTFWC peptide-PEG LysJBNP) and having the absorbance properties shown in FIG. 1D.

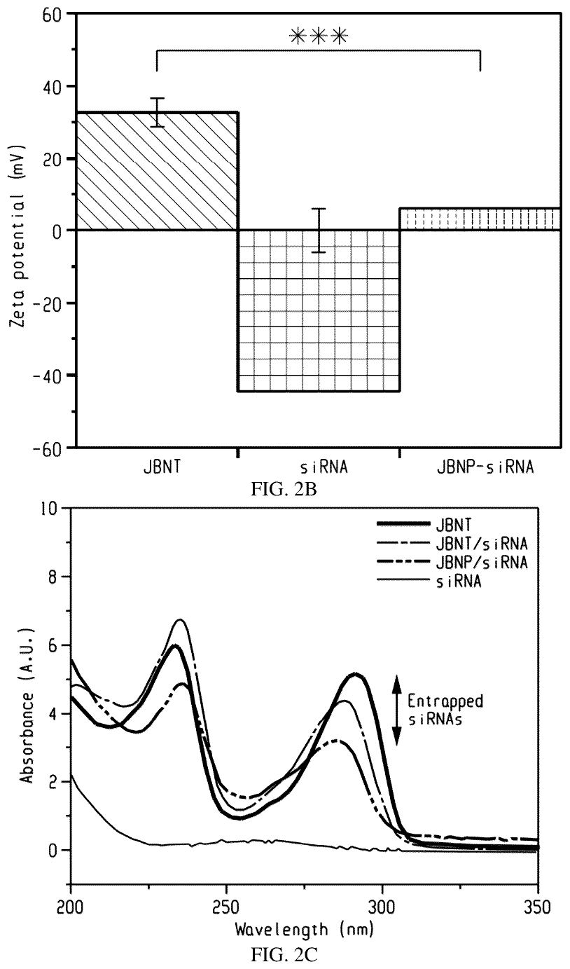

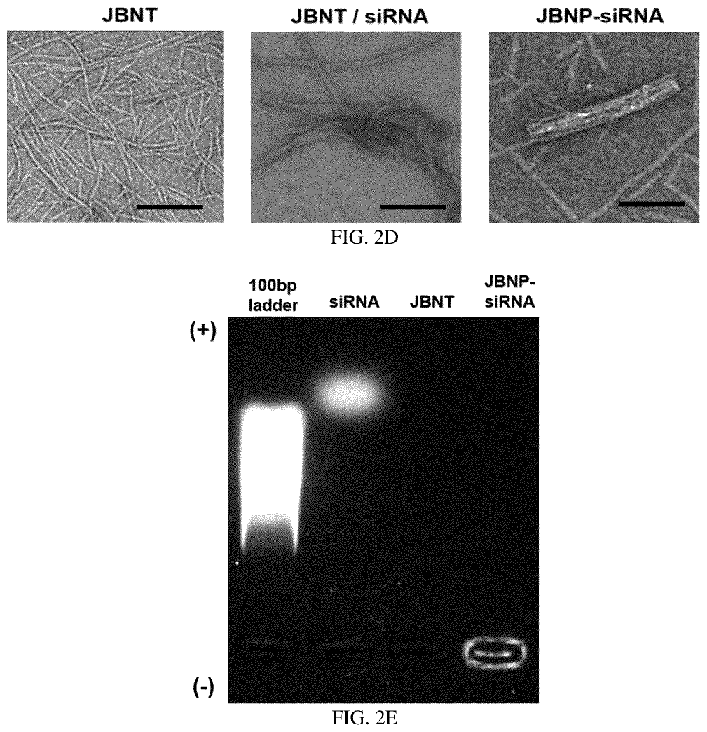

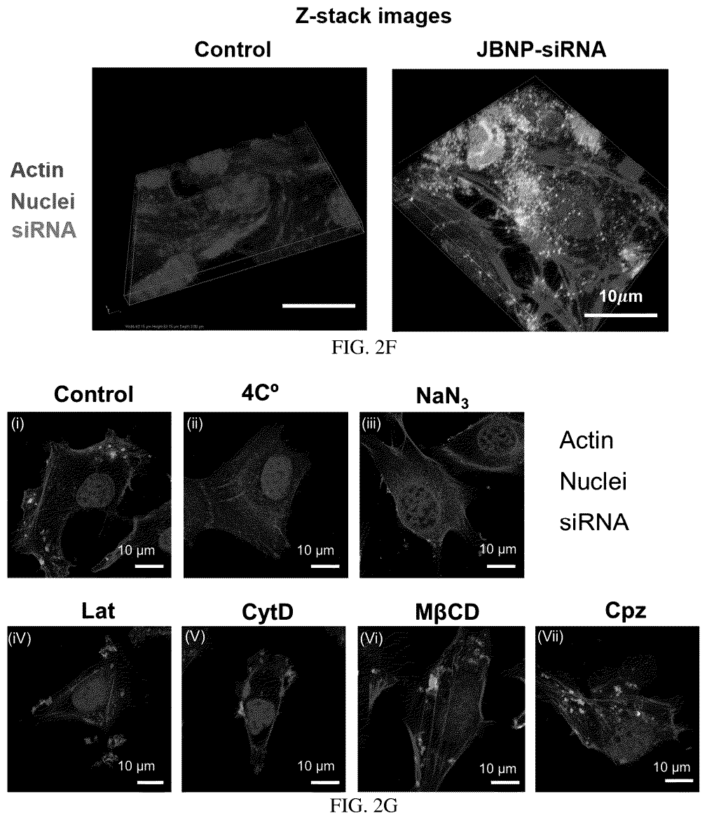

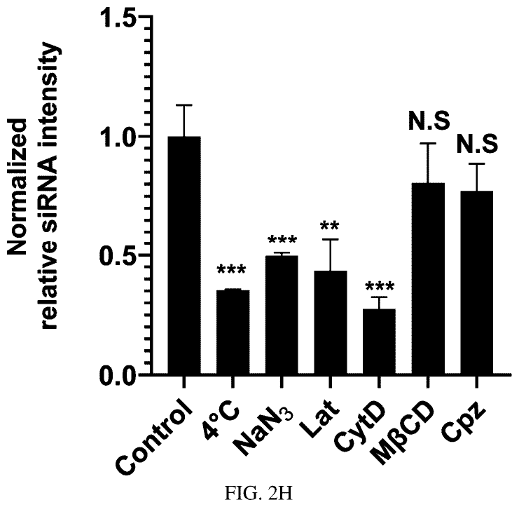

As shown in FIGS. 2A-2H, the JBNP includes a biologically active molecule as cargo, such as siRNA, protein, and/or small molecule drug, and can be used to deliver the biologically active molecules to target cells in vitro or in vivo. FIG. 2G-2H show the effects of various inhibitors on the uptake of the JBNP by target cell. Cells exposed to low temperature (4° C.), or treated with NaN₃, latrunculin-A (Lat), or Cytochalasin D (CytD), demonstrate decreased uptake of the JBNP as evidence by decreased intracellular fluorescence.

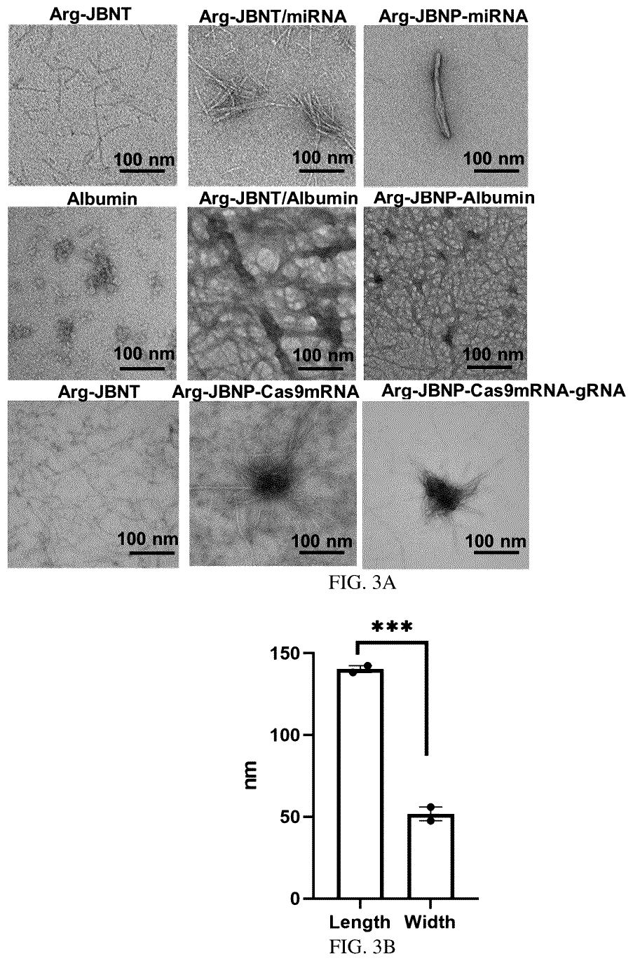

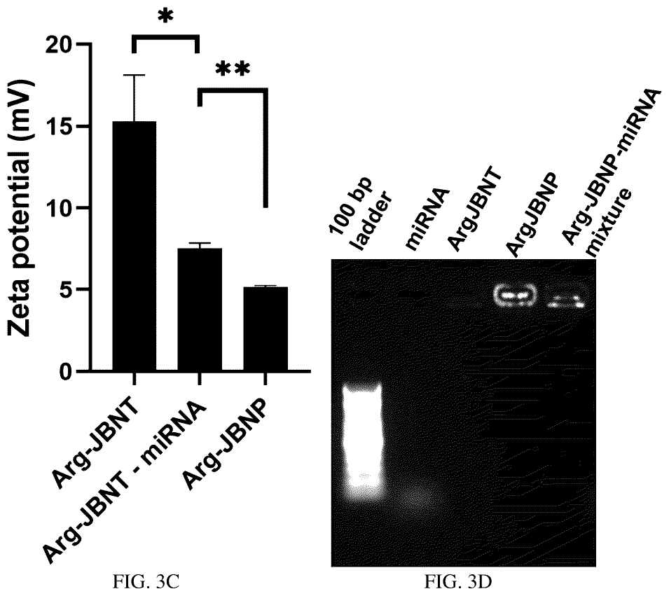

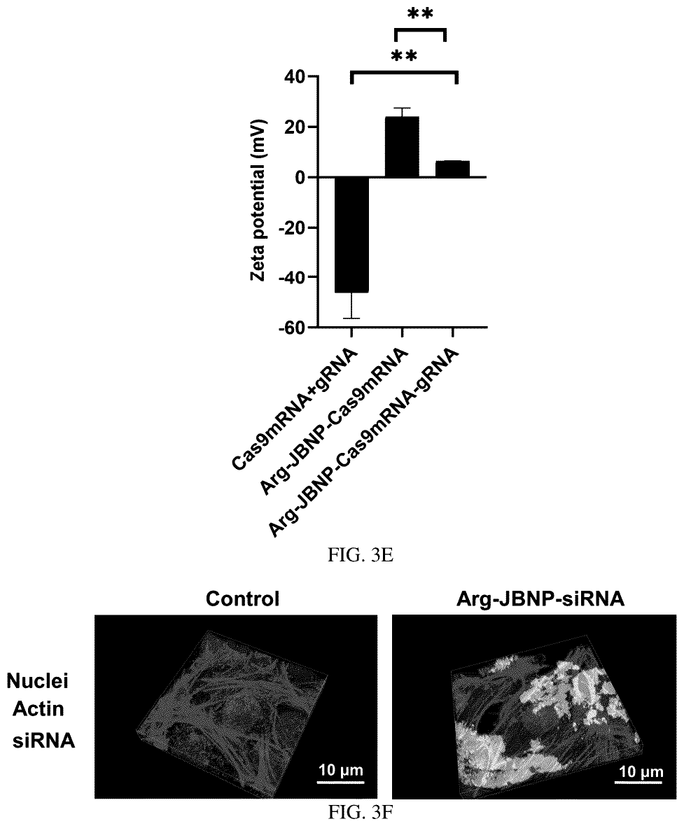

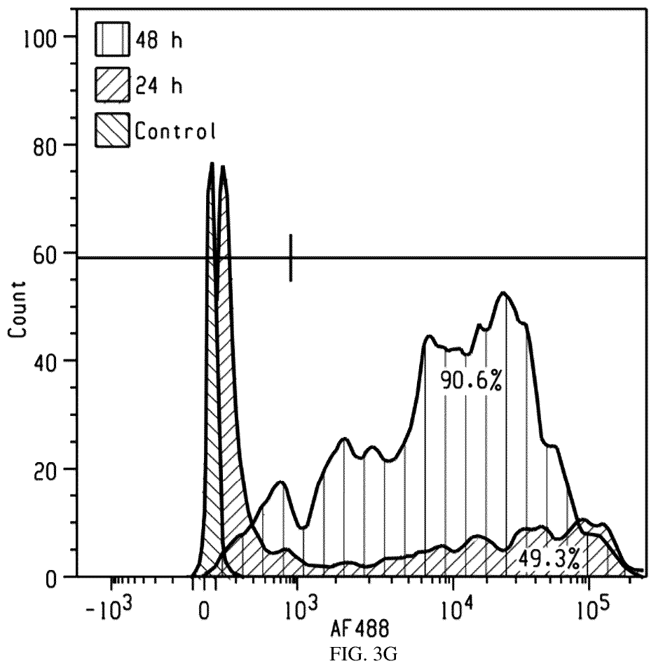

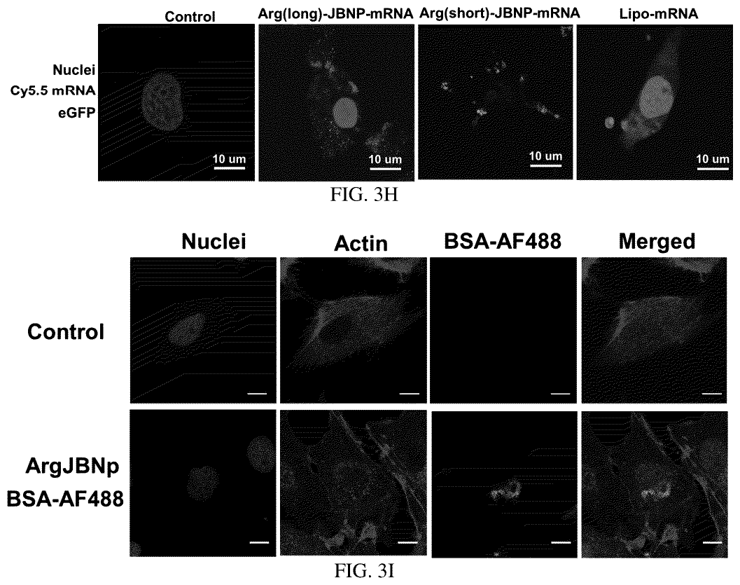

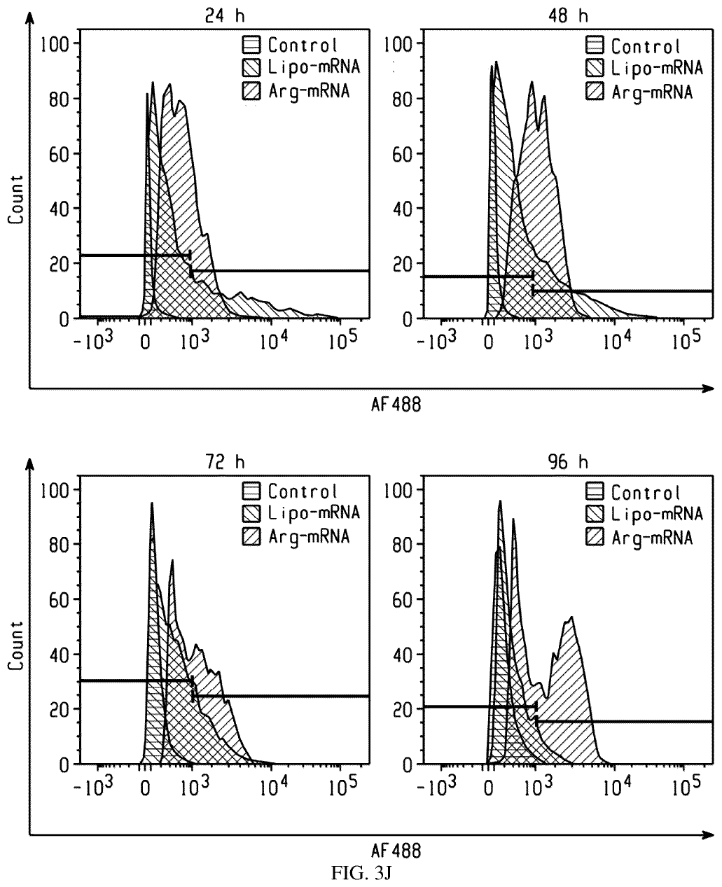

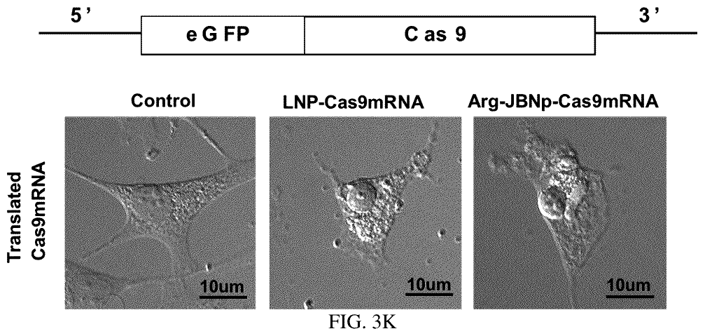

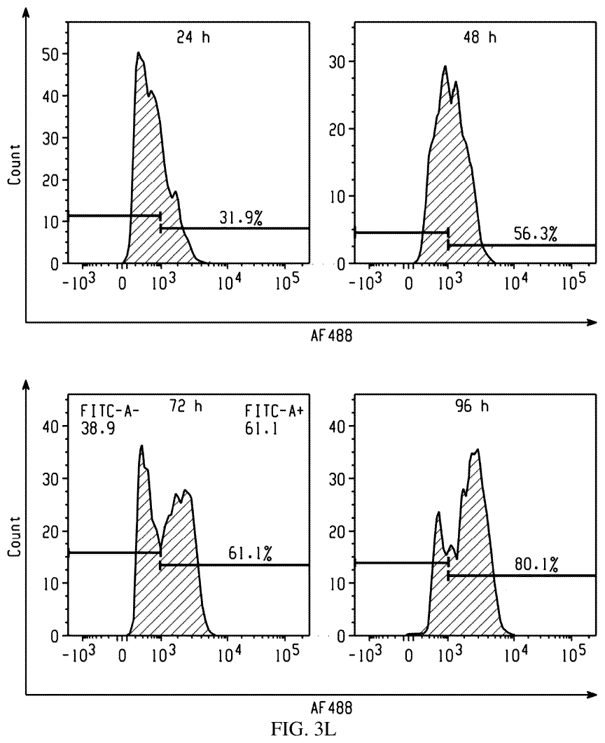

FIGS. 3A-3E shows the successful delivery of small RNAs, mRNAs, and albumin (protein) using ArgJBNTs. In FIG. 3A TEM images of Arg-JBNP containing miR140 (Arg-JBNP-miR140), Cas9 mRNA (Arg-JBNP-Cas9mRNA), and albumin (Arg-JBNP-Albumin). The results in FIG. 3B are based on analysis of TEM image data and show the average size and width of Arg-JBNP-miRNA. FIGS. 3C and 3E are a zeta potential analyses of Arg-JBNP-miR140 and Arg-JBNP-Cas9mRNA, respectively. FIG. 3D show the results of a gel retardation assay, while FIG. 3F shows CLSM z-stack images of siRNA-AF488 delivered by the Arg-JBNPs,

FIG. 3G is a flow cytometry analysis graph showing uptake of siRNA-AF488 by cells. FIG. 3H is fluorescence images of Cy5.5-eGFP-mRNA delivery via Arg-JBNP or Lipofectamine® 200. FIG. 3I is fluorescence images of BSA-AF488 delivery via Arg-JBNP. FIG. 3J is a series of flow cytometry graphs showing the time-dependent uptake of Cas9eGFP.

mRNA including the sequence encoding GFP upstream of Cas9 (see FIG. 3K) was added to Arg-JBNP to create Arg-JBNP-eGFP-Cas9mRNA. Fluorescence images showing uptake of Arg-JBNP-eGFP-Cas9mRNA by cells in shown in FIG. 3K. The time-dependent delivery of Arg-JBNP-eGFP-Cas9mRNA as measured by flow cytometry is shown in FIG. 3L.

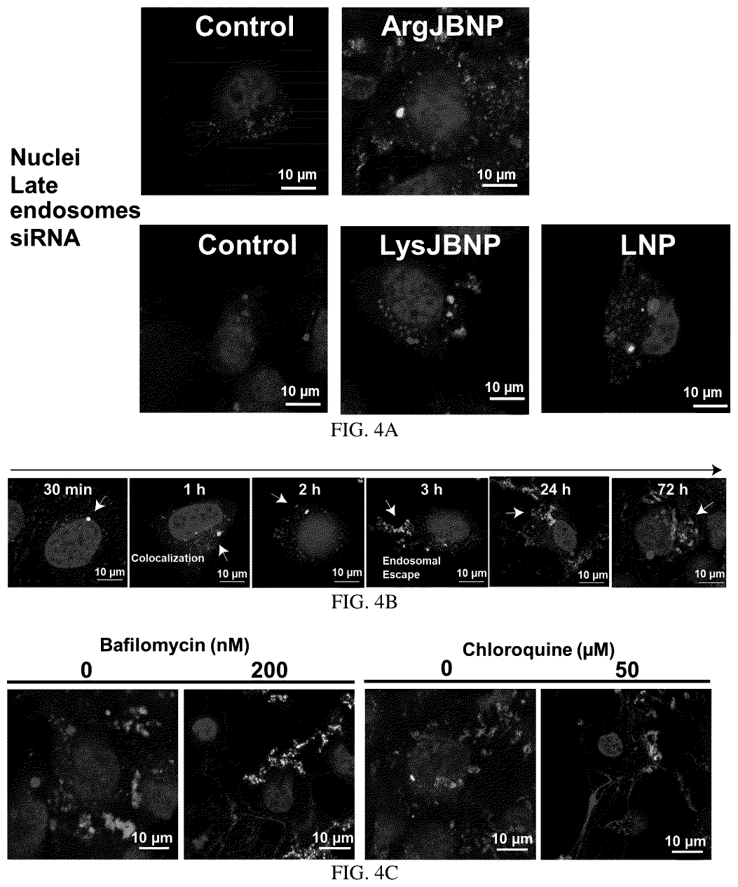

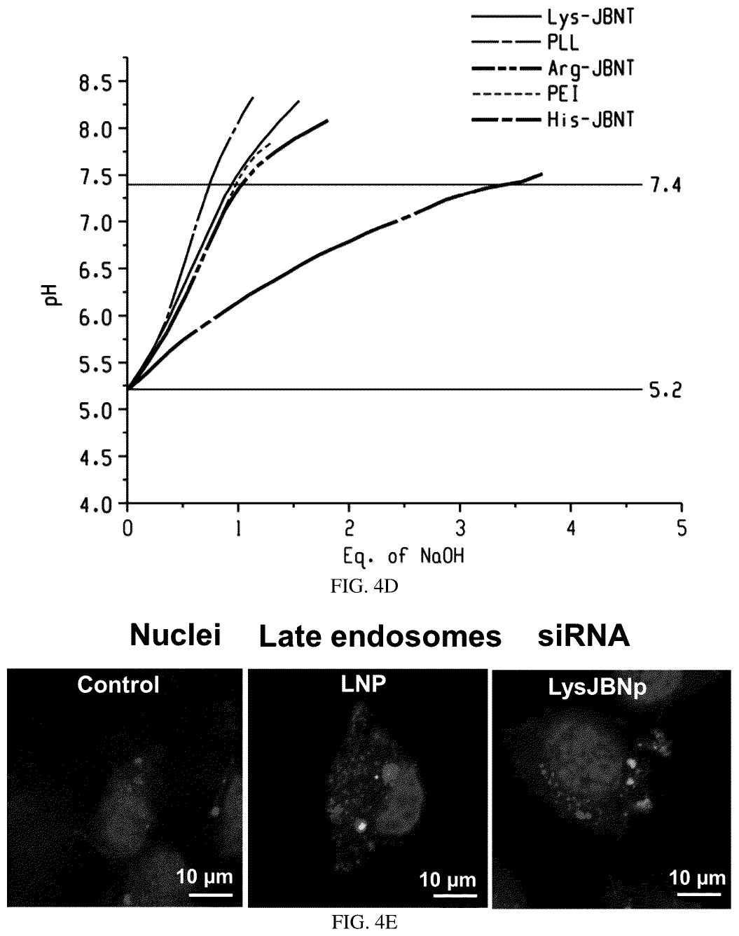

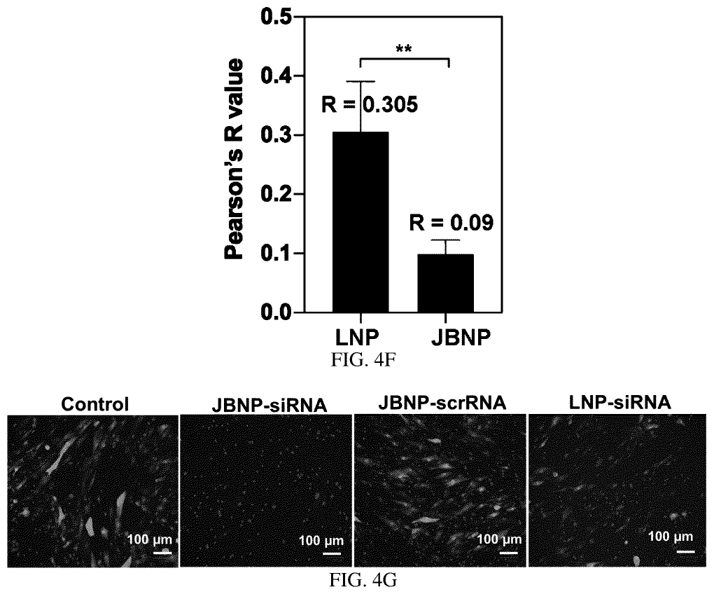

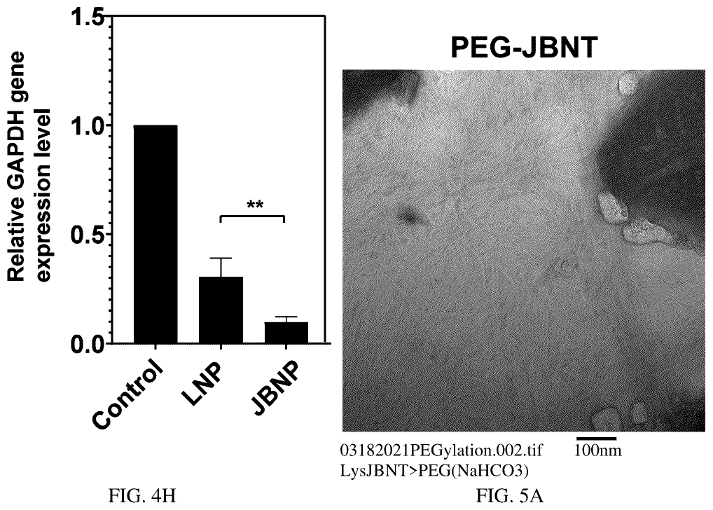

It was also demonstrated that the JBNPs have enhanced endosomal escape properties compared to lipid nanoparticles that are excellent for delivery of cargo biologically active molecules. The results are shown in FIGS. 4A-4G.

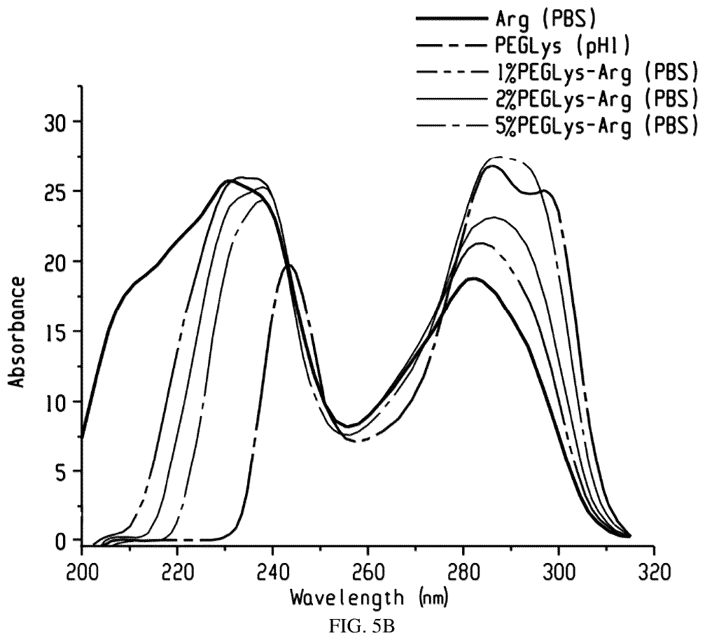

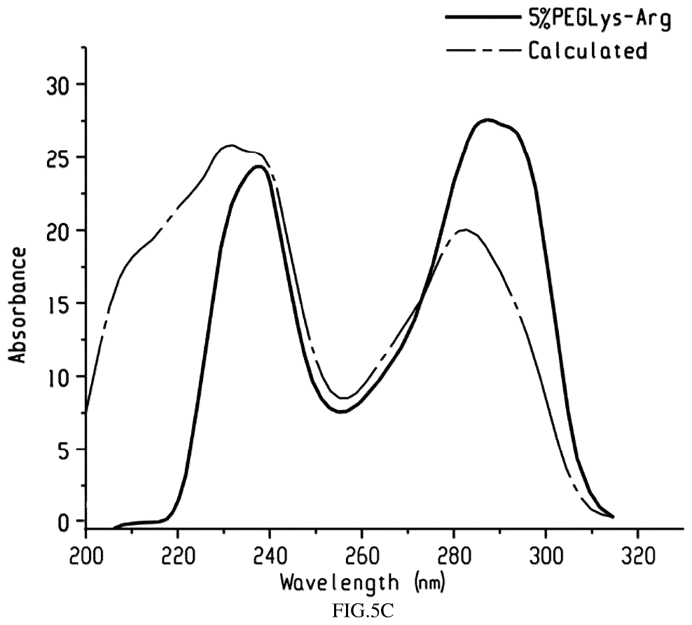

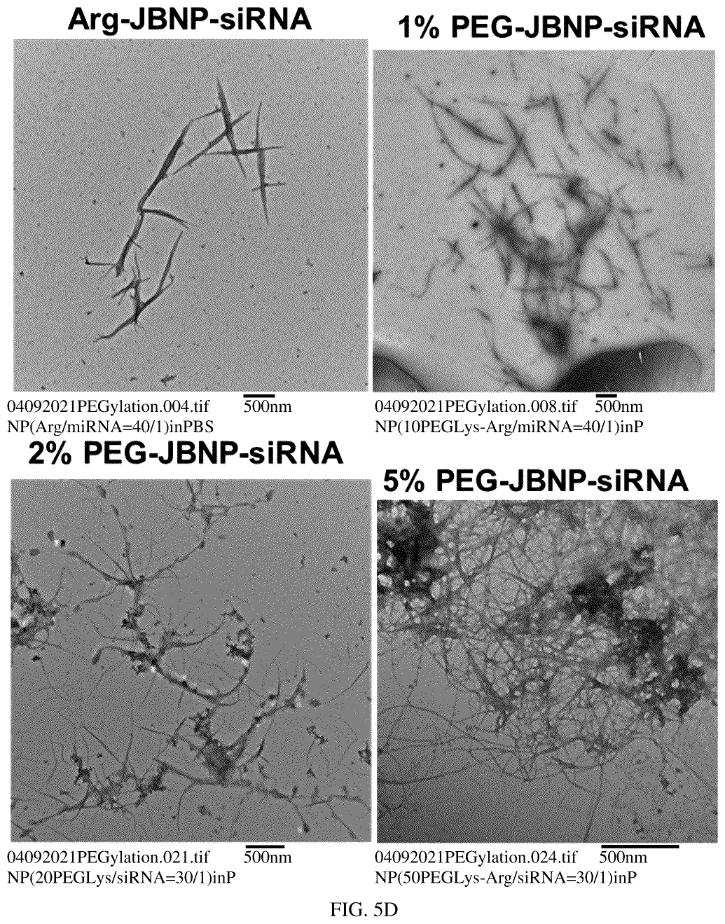

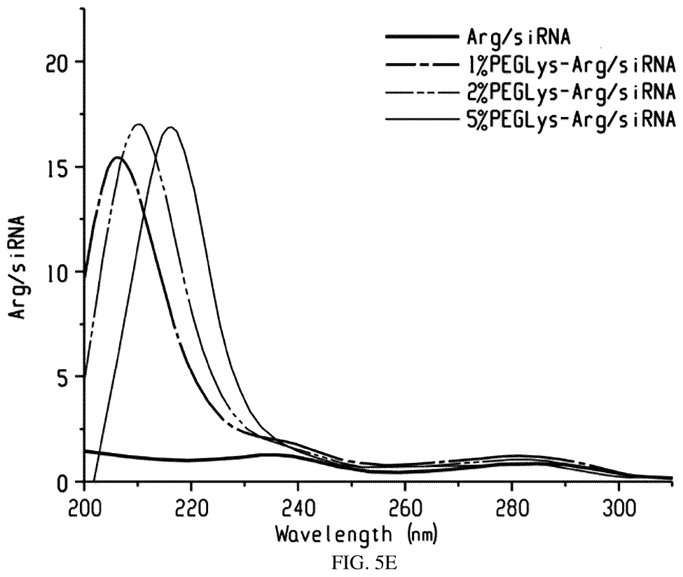

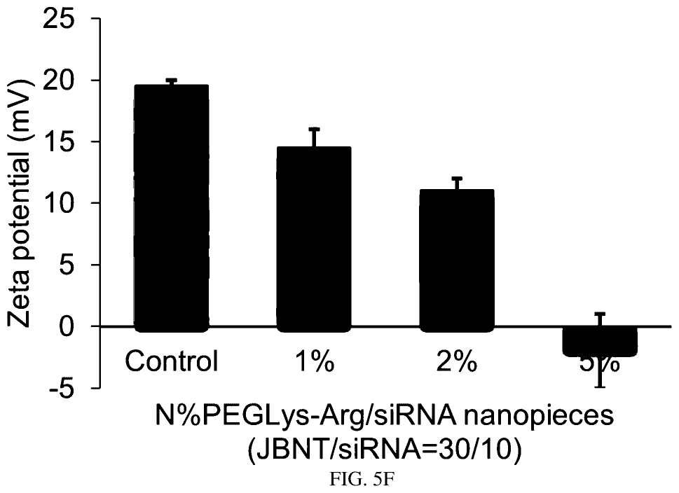

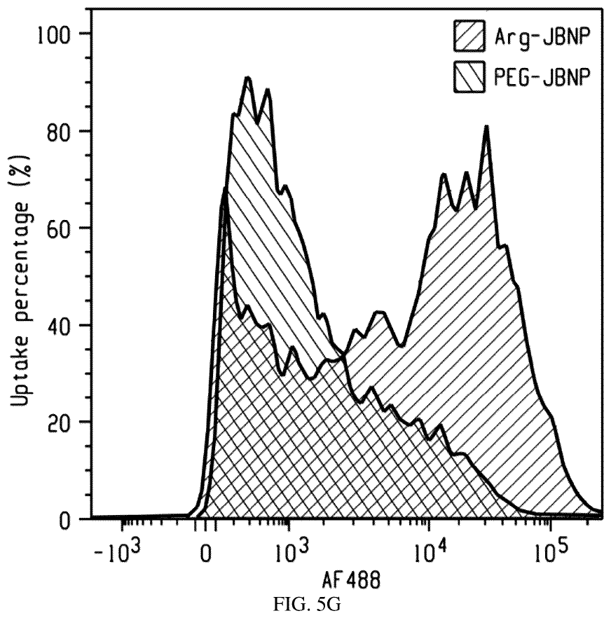

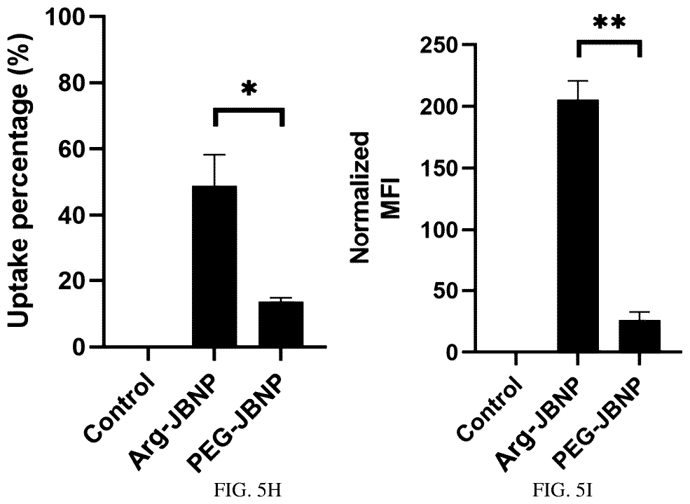

Moreover, as shown in FIGS. 5A-5H, JBNP modified with PEG are protected from non-specific cellular uptake by undesired types of cells. FIG. 5A shows the nanotube structure of PEGLysJBNT in TEM. FIGS. 5B and C show different ratios (0, 0.01:1, 0.02:1, 0.05:1) of PEGLysJBNT/ArgJBNT were compared in UV-Vis. FIG. 5D shows different ratios of (0, 0.01:1:0.33, 0.02:1:0.33, 0.05:1:0.33) PEGLysJBNT/ArgJBNT/siRNA were compared in TEM. FIG. 5E shows different ratios of (0, 0.01:1:0.33, 0.02:1:0.33, 0.05:1:0.33)PEGLysJBNT/ArgJBNT/siRNA were compared in UV-Vis. FIG. 5F shows different ratios of (0, 0.01:1:0.33, 0.02:1:0.33, 0.05:1:0.33)PEGLysJBNT/ArgJBNT/siRNA were compared by zeta-potential using zetasizer. In FIG. 5G we transfected the ArgJBNP or PEG-JBNP for 24 h and then performed flow-cytometry. FIGS. 5H and 5I show the analysis by flow-cytometry. PEGylation is to protect from non-specific cellular uptake (such as immune cells and other undesired cells) and then we want to add a targeting peptide with the PEG, so that we can achieve active targeting.

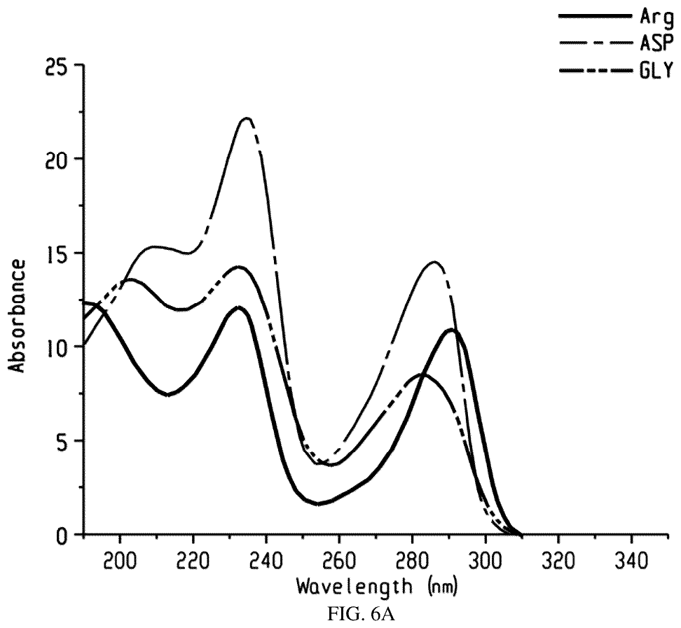

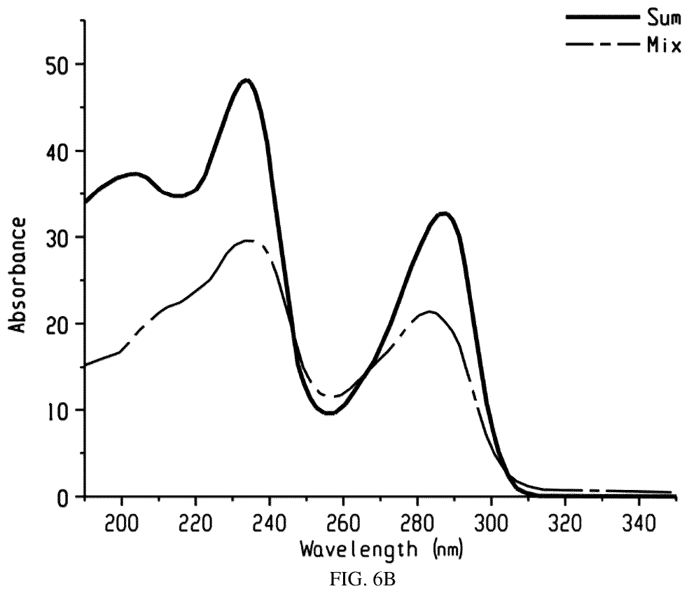

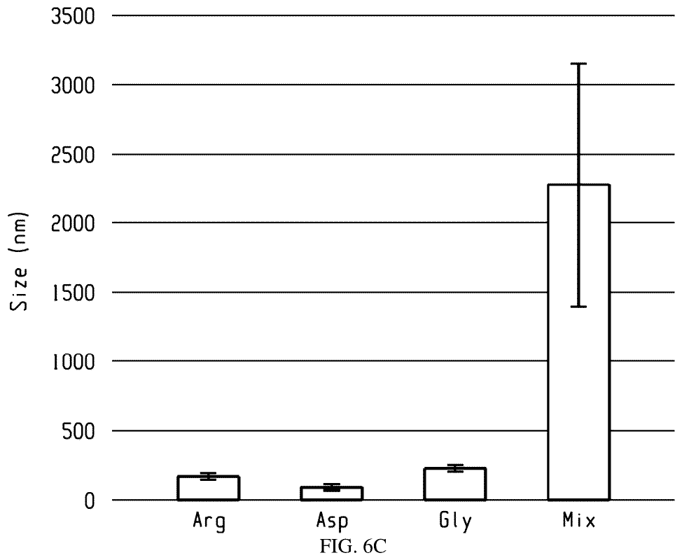

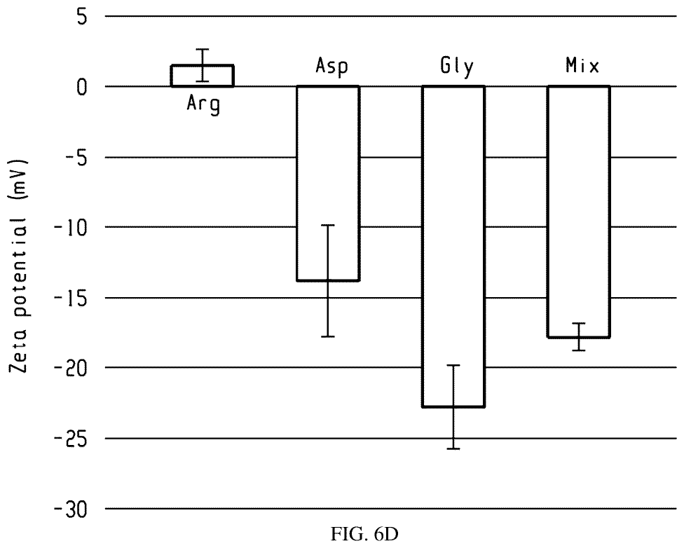

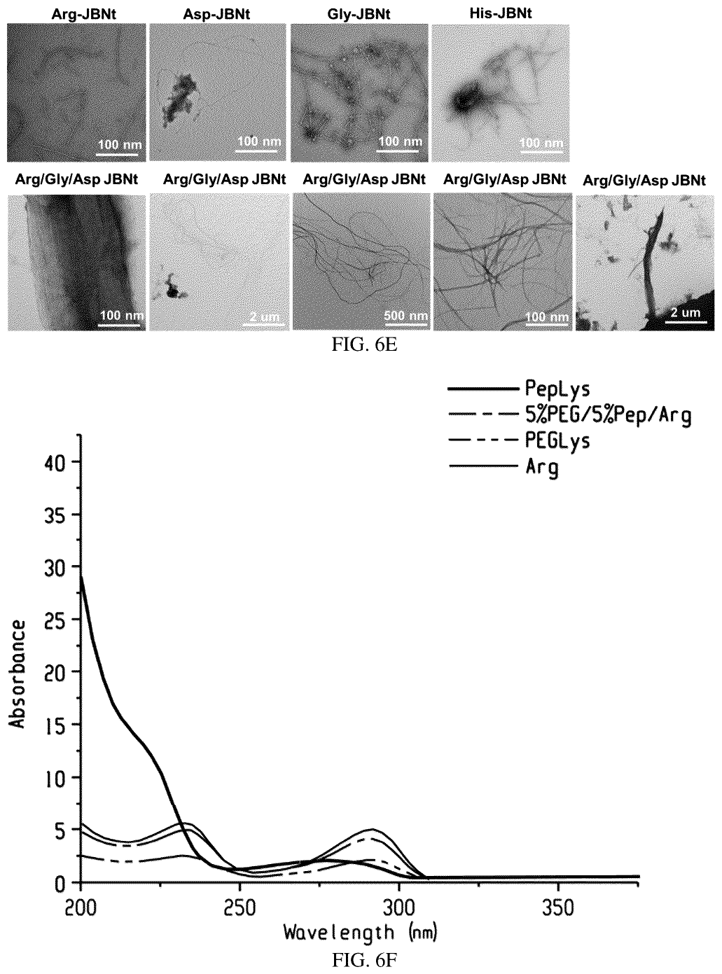

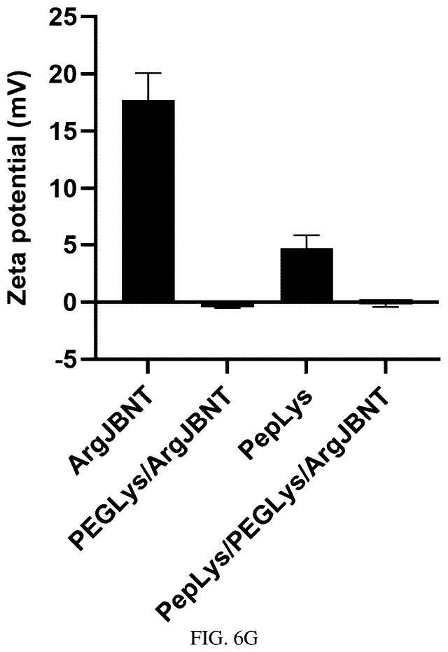

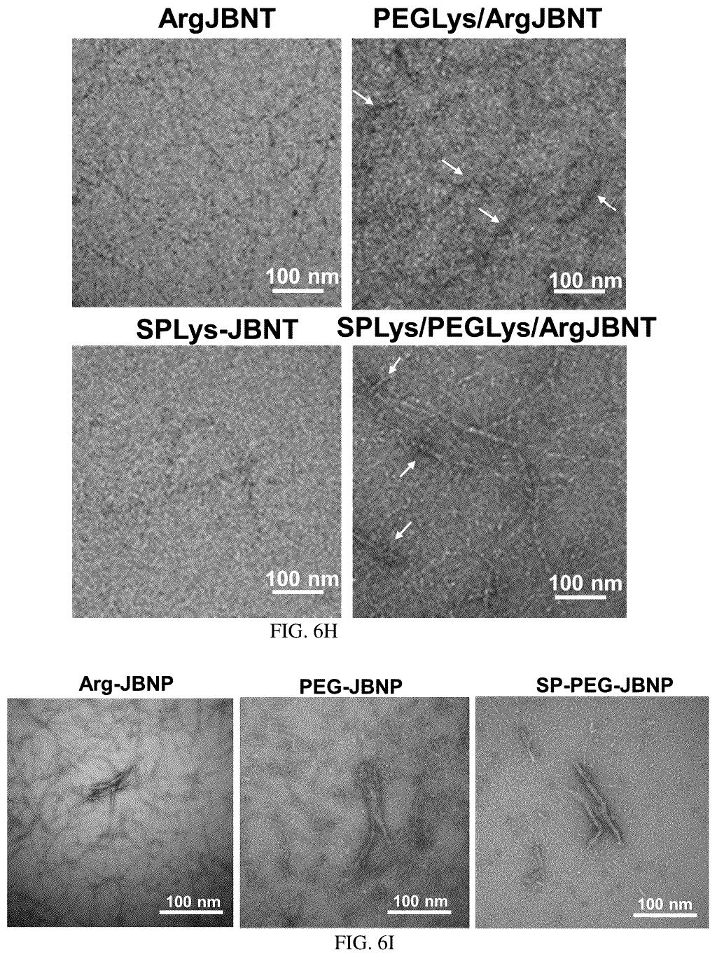

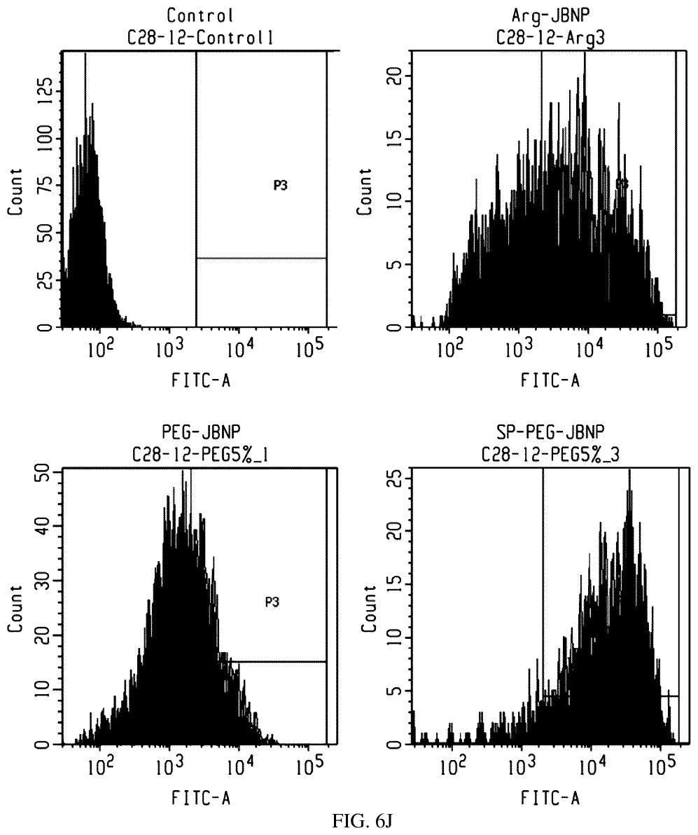

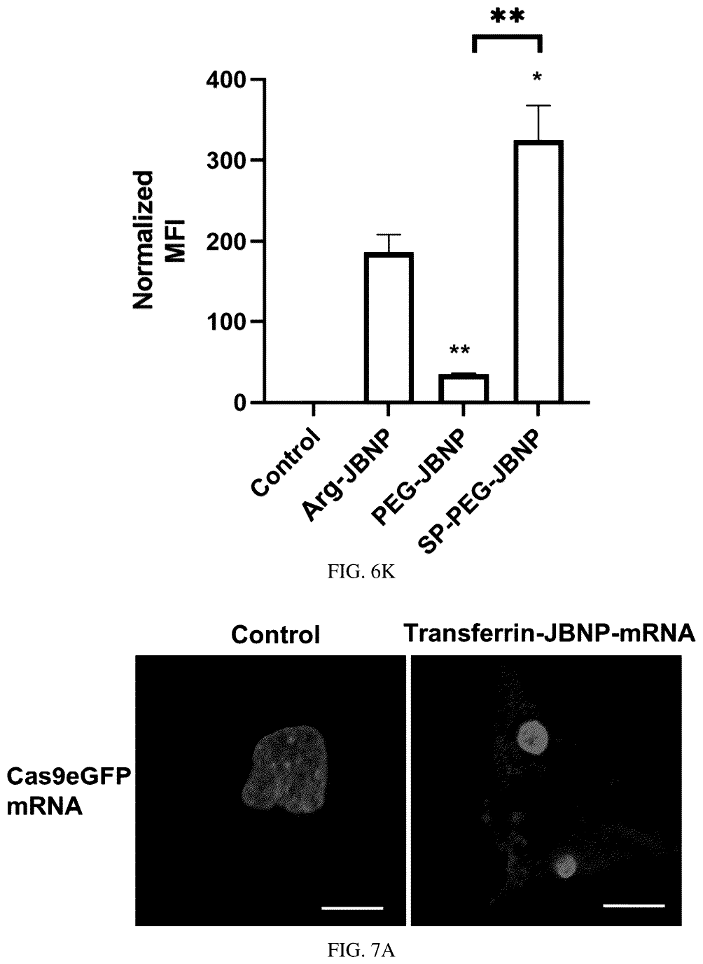

It was also demonstrated that combining multiple JBNTs having differing functions (i.e., multi-functional JBNT) provided multi-functional properties to the JBNTs. For example, it was possible to combine both the PEG effect and targeting ability of JBNPs. (See FIGS. 6A-6K). It can also be possible to combine to more positive charge JBNT such as Arginine-JBNT to increase the cell uptake efficiency. (See 3F-3L) Another example could be combining enhanced endosomal escape effect to the JBNT. This can be achieved by combining Histidine-JBNTs which has superior proton-sponge effect capacity (see FIG. 4D).

### Example 10: Examining the Ability of Cartilage-Targeting-JBNP-IL-1RA mRNA to Inhibit Osteoarthritis-Related Inflammation in the Knee Joint

Osteoarthritis (OA) severity is assessed by MANKIN score prior to administering treatment (saline, JBNP-IL-1RA mRNA, JBNP-scrambled mRNA, JBNP-without mRNA) via an intra-articular injection into surgical destabilization of the medial meniscus (DMM) model (3-month-old male) mice 1-month post-surgery. Safranin-O staining is performed on histological sections and examined via fluorescent microscopy. The following behavioral studies are performed to examine treatment efficacy: Y-Maze Spontaneous Alteration, Open Field, Object Recognition Test, and Fear Conditioning. An exemplary, nonlimiting example of an, mRNA sequence for IL-1RA is:

(SEQ ID NO: 87)
ATGGAAATCTGCAGAGGCCTCCGCAGTCACCTAATCACTCTCCTCCTCT
TCCTGTTCCATTCAGAGACGATCTGCCGACCCTCTGGGAGAAAATCCAG
CAAGATGCAAGCCTTCAGAATCTGGGATGTTAACCAGAAGACCTTCTAT
CTGAGGAACAACCAACTAGTTGCTGGATACTTGCAAGGACCAAATGTCA
ATTTAGAAGAAAAGATAGATGTGGTACCCATTGAGCCTCATGCTCTGTT
CTTGGGAATCCATGGAGGGAAGATGTGCCTGTCCTGTGTCAAGTCTGGT
GATGAGACCAGACTCCAGCTGGAGGCAGTTAACATCACTGACCTGAGCG
AGAACAGAAAGCAGGACAAGCGCTTCGCCTTCATCCGCTCAGACAGTGG
CCCCACCACCAGTTTTGAGTCTGCCGCCTGCCCCGGTTGGTTCCTCTGC
ACAGCGATGGAAGCTGACCAGCCCGTCAGCCTCACCAATATGCCTGACG
AAGGCGTCATGGTCACCAAATTCTACTTCCAGGAGGACGAGTAG.

### Example 11: Examining the Ability of Cartilage-Targeting-JBNP-IL-1RA Peptide mRNA to Inhibit Osteoarthritis-Related Inflammation in the Knee Joint

OA severity is assessed by MANKIN score prior to administrating treatment (saline, JBNP-IL-1RA peptide mRNA, JBNP-scrambled mRNA, JBNP-without mRNA) via an intra-articular injection into surgical destabilization of the medial meniscus (DMM) model (3-month-old) mice 1-month post-surgery. The IL-1RA peptide is an interleukin-1 receptor antagonist peptide. Safranin-O staining is performed on histological sections and examined via fluorescent microscopy. The following behavioral studies are performed to examine treatment efficacy: Y-Maze Spontaneous Alteration, Open Field, Object Recognition Test, and Fear Conditioning. An exemplary, nonlimiting example of an, mRNA sequence for an IL-1RA peptide is:

(SEQ ID NO: 88)
ATGCGGCCTAGCGGCAGAAAGTCTAGCAAGATGCAGGCCTTTAGAATCT
GGGACGTTAATCAGAAAACCTTCTACCTCAGAAACAACCAGCTGGTCGCC
GGCTACCTGCAGGGCCCCAACGTGAATCTGGAAGAGAAGATCGACGTGG
TGCCAATCGAGCCTCACGCCCTGTTTCTGGGCATCCACGGCGGAAAAAT
GTGCCTGTCTTGTGTGAAGTCCGGAGATGAGACAAGACTGCAACTGGAA
GCCGTGAACATTACAGACCTGAGCGAGAACCGGAAGCAGGACAAGCGGT
TCGCCTTCATCAGAAGCGACAGCGGCCCTACAACCAGCTTCGAGAGCGC
CGCTTGCCCCGGCTGGTTCCTGTGCACCGCCATGGAAGCTGATCAGCCT
GTGTCCCTGACCAACATGCCTGATGAAGGCGTGATGGTGACCAAGTTCT
ACTTCCAGGAGGACGAGATGTGA.

### Example 12: Amino Acid Side Change Modification can be Utilized to Target Self-Assembled Nanomaterials of the Present Disclosure to Organs of Interest

The protein corona of the self-assembled nanomaterials described herein and compositions comprising the same can be further modified through the modification of the side chain of the amino acid residue. Assembled JBNPs are incubated with mice serum for 30 minutes. Then, centrifuge for 15 minutes at 13800 relative centrifugal force (RCF) at 4° C. The supernatant is discarded, and pellet washed three times with phosphate-buffered saline. The proteins are separates via SDS-PAGE and the protein bands identified via Liquid Chromatography-tandem Mass spectrometry (LC/MS/MS). Lysine and arginine side chains bind Apolipoprotein E (APOE) family and believed to target the self-assembled nanomaterials of the present disclosure to the liver. By way of further example, it is believed that the histidine side chain in the protein corona composition targets the self-assembled nanomaterials of the present disclosure will target to organs other than liver.

### Example 13: JBNP of the Present Disclosure Actively Targets Cartilage and has Enhance Half-Life when a Cartilage Targeting Peptide WYRGRL is Attached to the JBNT

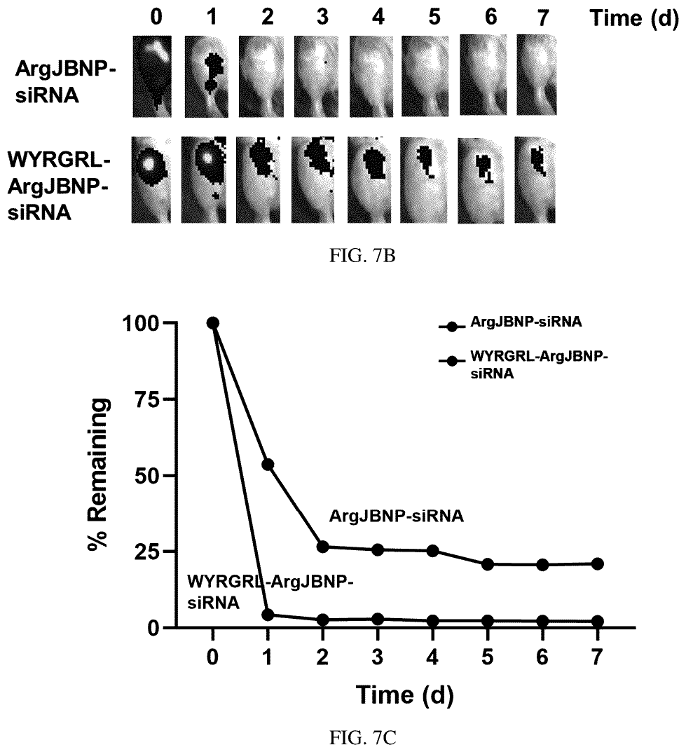

The cartilage targeting peptide WYRGRL (SEQ ID NO:47) was attached to JBNP with an siRNA reporter and the assembled WYRGRL-JBNP with the siRNA-dye was transferred to human chondrocytes C28/I2 cells. Fluorescence activated cell sorting (FACS) was performed to quantify the targeting ability of the WYRGRL-JBNP to target cartilage. The JBNP of the present disclosure actively targets cartilage and has enhance half-life when a cartilage-targeting molecule or moiety is attached thereto (FIG. 7B).

### Example 14: Examining the Ability of miR140 Delivery with Cartilage-Targeting-ArgJBNP to Slow Down or Prevent Osteoarthritis Progression

The cartilage-targeting can be accomplished, e.g., via peptide WYRGRL. OA severity is assessed by MANKIN score prior to administering treatment (saline, JBNP-miR140, JBNP-scrambled miRNA, or JBNP-without miR140) via an intra-articular injection into surgical destabilization of the medial meniscus (DMM) model mice 1-month post-surgery. The following behavioral studies are performed to examine treatment efficacy: Y-Maze Spontaneous Alteration, Open Field, Object Recognition Test, and Fear Conditioning. DAPI staining, H&E staining, and Safranin-O staining of histological sections is used to demonstrate effective targeting and delivery via fluorescent microscopy.

### Example 15: Examining the Ability of Cartilage-Targeting-JBNP-CRISPR Targeting sgIL-1R to Treat Osteoarthritis, Slow Down the Progression of Osteoarthritis, and Inhibit Inflammation

The cartilage-targeting can be accomplished, e.g., via peptide WYRGRL. OA severity is assessed by MANKIN score prior to administering treatment (saline, JBNP-CRISPR-sgIL-1R, JBNP-CRISPR-sgNeg, or JBNP-CRISPR-without sgRNA) via an intra-articular injection into surgical destabilization of the medial meniscus (DMM) model mice 2 months post-surgery or 129SVE-M wild-type mice. DAPI staining, H&E staining, and Safranin-O staining of histological sections examines OA progress via fluorescent microscopy. The following behavioral studies are performed to examine treatment efficacy: Y-Maze Spontaneous Alteration, Open Field, Object Recognition Test, and Fear Conditioning.

### Example 16: Examining the Ability of Cartilage-Targeting-JBNP-IL-1R siRNA to Inhibit Osteoarthritis-Related Inflammation in the Knee Joint

The cartilage-targeting can be accomplished, e.g., via peptide WYRGRL. OA severity is assessed by MANKIN score prior to administering treatment (saline, JBNP-IL-1R siRNA, JBNP-negative siRNA, JBNP-without siRNA) via an intra-articular injection into surgical destabilization of the medial meniscus (DMM) model (3-month-old male) mice 1-month post-surgery. Safranin-O staining is performed on histological sections and examined via fluorescent microscopy. The following behavioral studies are performed to examine treatment efficacy: Y-Maze Spontaneous Alteration, Open Field, Object Recognition Test, and Fear Conditioning.

### Example 17: Examining the Ability of Cartilage-Targeting JBNP-IL-1RA mRNA to Inhibit Osteoarthritis-Related Inflammation in the Knee Joint

The cartilage-targeting can be accomplished, e.g., via peptide WYRGRL. OA severity is assessed by MANKIN score prior to administering treatment (saline, JBNP-IL-1RA mRNA, JBNP-scrambled mRNA, JBNP-without mRNA) via an intra-articular injection into surgical destabilization of the medial meniscus (DMM) model (3-month-old male) mice 1-month post-surgery. Safranin-O staining is performed on histological sections and examined via fluorescent microscopy. The following behavioral studies are performed to examine treatment efficacy: Y-Maze Spontaneous Alteration, Open Field, Object Recognition Test, and Fear Conditioning. An exemplary, nonlimiting example of an mRNA sequence for IL-1RA is:

(SEQ ID NO: 89)
ATGGAAATCTGCAGAGGCCTCCGCAGTCACCTAATCACTCTCCTCCTCT
TCCTGTTCCATTCAGAGACGATCTGCCGACCCTCTGGGAGAAAATCCAG
CAAGATGCAAGCCTTCAGAATCTGGGATGTTAACCAGAAGACCTTCTAT
CTGAGGAACAACCAACTAGTTGCTGGATACTTGCAAGGACCAAATGTCA
ATTTAGAAGAAAAGATAGATGTGGTACCCATTGAGCCTCATGCTCTGTT
CTTGGGAATCCATGGAGGGAAGATGTGCCTGTCCTGTGTCAAGTCTGGT
GATGAGACCAGACTCCAGCTGGAGGCAGTTAACATCACTGACCTGAGCG
AGAACAGAAAGCAGGACAAGCGCTTCGCCTTCATCCGCTCAGACAGTGG
CCCCACCACCAGTTTTGAGTCTGCCGCCTGCCCCGGTTGGTTCCTCTGC
ACAGCGATGGAAGCTGACCAGCCCGTCAGCCTCACCAATATGCCTGACG
AAGGCGTCATGGTCACCAAATTCTACTTCCAGGAGGACGAGTAG.

### Example 18: Examining the Ability of Cartilage-Targeting JBNP-IL-1RA Peptide mRNA to Inhibit Osteoarthritis Inflammation in the Knee Joint

The cartilage-targeting can be accomplished, e.g., via peptide WYRGRL. OA severity is assessed by MANKIN score prior to administrating treatment (saline, JBNP-IL-1RA peptide mRNA, JBNP-scrambled mRNA, JBNP-without mRNA) via an intra-articular injection into surgical destabilization of the medial meniscus (DMM) model (3-month-old) mice 1-month post-surgery. The IL-1RA peptide is an interleukin-1 receptor antagonist peptide. Safranin-O staining is performed on histological sections and examined via fluorescent microscopy. The following behavioral studies are performed to examine treatment efficacy: Y-Maze Spontaneous Alteration, Open Field, Object Recognition Test, and Fear Conditioning. An exemplary, nonlimiting example of an, mRNA sequence for an IL-1RA peptide is:

(SEQ ID NO: 90)
ATGCGGCCTAGCGGCAGAAAGTCTAGCAAGATGCAGGCCTTTAGAATCT
GGGACGTTAATCAGAAAACCTTCTACCTCAGAAACAACCAGCTGGTCGC
CGGCTACCTGCAGGGCCCCAACGTGAATCTGGAAGAGAAGATCGACGTG
GTGCCAATCGAGCCTCACGCCCTGTTTCTGGGCATCCACGGCGGAAAAA
TGTGCCTGTCTTGTGTGAAGTCCGGAGATGAGACAAGACTGCAACTGGA
AGCCGTGAACATTACAGACCTGAGCGAGAACCGGAAGCAGGACAAGCGG
TTCGCCTTCATCAGAAGCGACAGCGGCCCTACAACCAGCTTCGAGAGCG
CCGCTTGCCCCGGCTGGTTCCTGTGCACCGCCATGGAAGCTGATCAGCC
TGTGTCCCTGACCAACATGCCTGATGAAGGCGTGATGGTGACCAAGTTC
TACTTCCAGGAGGACGAGATGTGA.

### Example 19: Amino Acid Side Change Modification can be Utilized to Target Self-Assembled Nanomaterials of the Present Disclosure to Organs of Interest

The protein corona of the self-assembled nanomaterials described herein and compositions comprising the same can be further modify through the modification of the side chain of the amino acid residue. Assembled JBNPs are incubated with mice serum for 30 minutes. Then, centrifuge for 15 minutes at 13800 RCF at 4° C. The supernatant is discarded, and pellet washed three times with phosphate-buffered saline. The proteins are separates via SDS-PAGE and the protein bands identified via Liquid Chromatography-tandem Mass spectrometry (LC/MS/MS). Lysine and arginine side chains bind Apolipoprotein E (APOE) family and believed to target the self-assembled nanomaterials of the present disclosure to the liver. By way of further example, it is believed that the histidine side chain in the protein corona composition targets the self-assembled nanomaterials of the present disclosure will target to organs other than liver.

### Example 20: JBNP of the Present Disclosure Actively Targets Cartilage and has Enhance Half-Life when Cartilage Targeting Peptide WYRGRL is Attached to the JBNT

The cartilage targeting peptide WYRGRL (SEQ ID NO:47) was attached to JBNP with an mRNA reporter and the assembled WYRGRL-JBNP with the mRNA-dye was transferred to human chondrocytes C28/I2 cells. FACS was performed to quantify the targeting ability of the WYRGRL-JBNP to target cartilage. The JBNP actively targeted cartilage and had an enhance half-life when a cartilage-targeting molecule or moiety was attached thereto (FIG. 7B and FIG. 7C)

### Example 21: Liver-Targeting-JBNP of the Present Disclosure can Deliver an Agent to the Liver

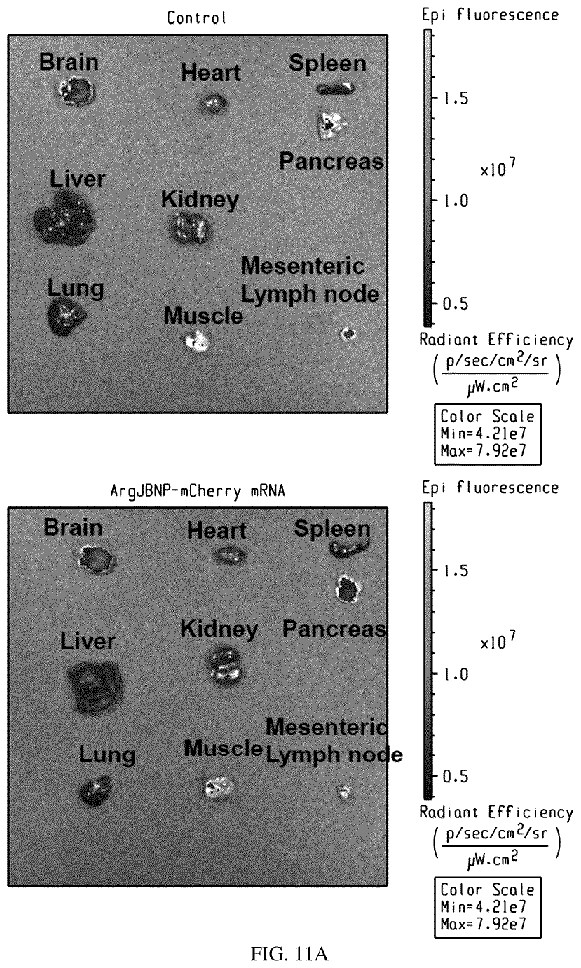

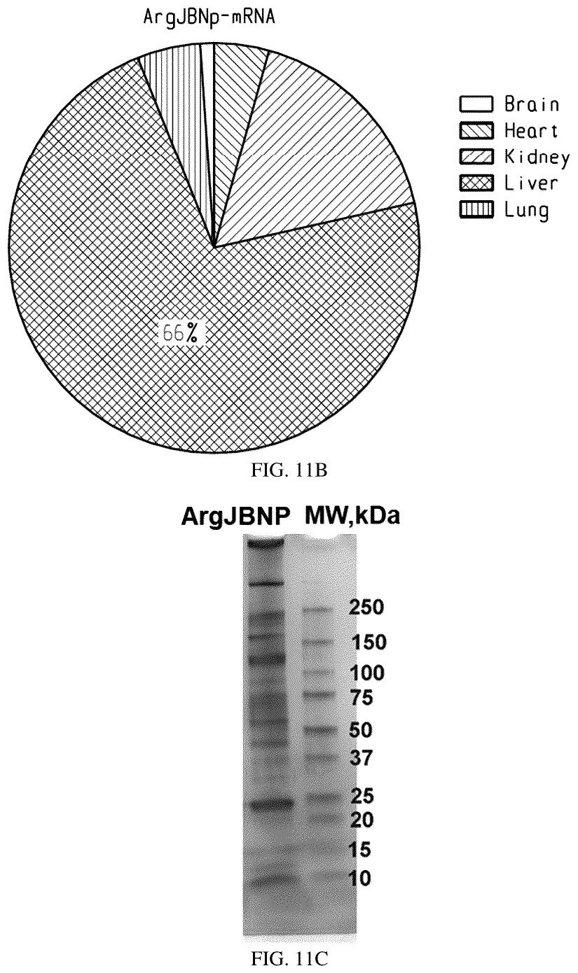

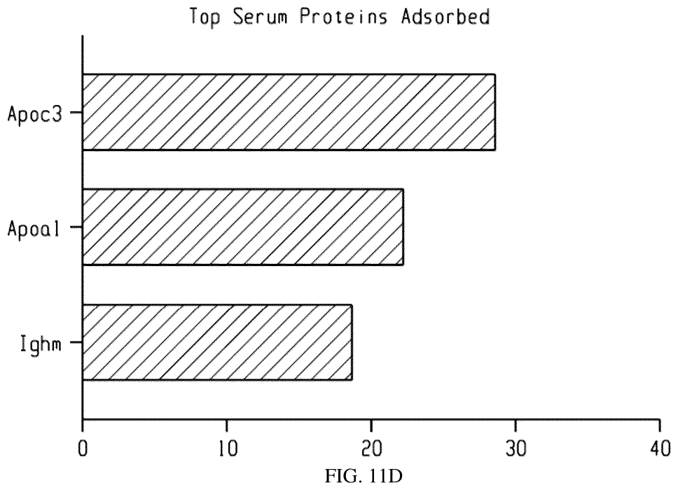

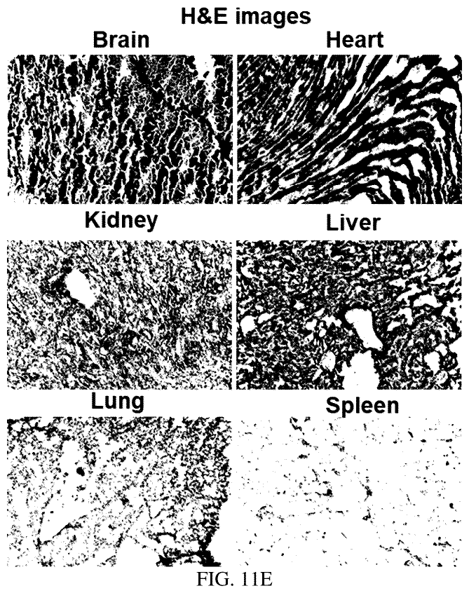

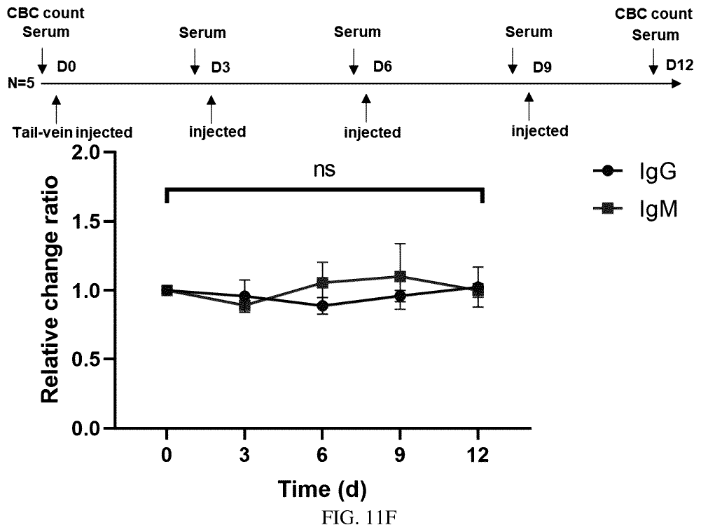

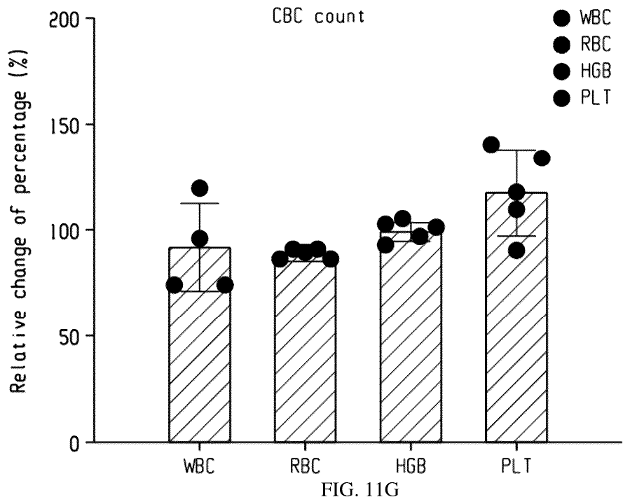

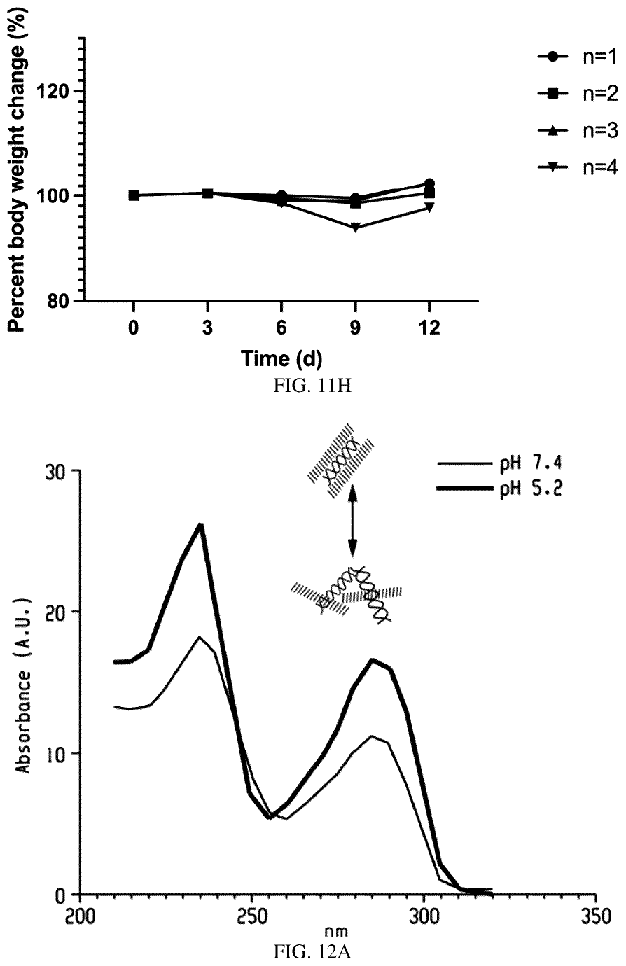

ArgJBNP-mcherry mRNA was intravenously administered to BALB/cJ mice. Biodistribution of the ArgJBNP-mcherry mRNA was monitored via IVIS® imaging (FIG. 11A and FIG. 11B). ArgJBNP has significant biodistribution to the liver. The transfected liver can then be homogenized. Western blot and real-time reverse transcriptase quantitative polymerase chain reaction (RT-qPCR) to determine the transfection efficiency.

### Example 22: Examining the Ability of Kidney-Targeting-JBNP of the Present Disclosure to Deliver an Agent to the Kidney

Kidney-targeting-JBNP-mRNA Cy5 is intravenously administered to BALB/cJ mice. Biodistribution of the Kidney-targeting-JBNP-mRNA Cy5 is monitored via IVIS® imaging. Then, the transfected kidney is homogenized. Western blot and RT-qPCR was used to determine the transfection efficiency.

### Example 23: Examining the Ability of Brain-Targeting-JBNP of the Present Disclosure to Deliver an Agent to the Brain

Brain-targeting-JBNP-mRNA Cy5 is intravenously administered to BALB/cJ mice. Biodistribution of the Brain-targeting-JBNP-mRNA Cy5 is monitored via IVIS® imaging. Then, the transfected brain is homogenized. Western blot and RT-qPCR are used to determine the transfection efficiency.

### Example 24: Examining the Ability of Lung-Targeting-JBNP of the Present Disclosure to Deliver an Agent to the Lung

Lung-targeting-JBNP-mRNA Cy5 is intravenously administered to BALB/cJ mice. Biodistribution of the Lung-targeting-JBNP-mRNA Cy5 is monitored via IVIS® imaging. Then, the transfected lung is homogenized. Western blot and RT-qPCR are used to determine the transfection efficiency.

### Example 25: Examining the Ability of Spleen-Targeting-JBNP of the Present Disclosure to Deliver an Agent to the Spleen and Lymph Nodes

Spleen-targeting-JBNP-mRNA Cy5 is intravenously administered to BALB/cJ mice. Biodistribution of the Spleen-targeting-JBNP-mRNA Cy5 is monitored via IVIS® imaging. Then, the transfected spleen and lymph nodes are homogenized. Western blot and RT-qPCR are used to determine the transfection efficiency.

### Example 26: Examining the Ability of Bone-Targeting-JBNP of the Present Disclosure to Deliver an Agent to Bone

Bone-targeting-JBNP-mRNA Cy5 is intravenously administered to BALB/cJ mice. Biodistribution of the Bone-targeting-JBNP-mRNA Cy5 is monitored via IVIS® imaging. Then, the transfected bone is homogenized. Western blot and RT-qPCR are used to determine the transfection efficiency.

### Example 27: Examining the Ability of Muscle-Targeting-JBNP of the Resent Disclosure to Deliver an Agent to Muscle

Muscle-targeting-JBNP-mRNA Cy5 is intravenously administered to BALB/cJ mice. Biodistribution of the Muscle-targeting-JBNP-mRNA Cy5 is monitored via IVIS® imaging. Then, the transfected muscle is homogenized. Western blot and RT-qPCR are used to determine the transfection efficiency.

### Example 28: Examining the Ability of Heart-Targeting-JBNP of the Present Disclosure to Deliver an Agent to the Heart

Heart-targeting-JBNP-mRNA Cy5 is intravenously administered to BALB/cJ mice. Biodistribution of Heart-targeting-JBNP-mRNA Cy5 is monitored via IVIS® imaging. Then, the transfected heart is homogenized. Western blot and RT-qPCR are used to determine the transfection efficiency.

### Example 29: Examining the Ability of Pancreas-Targeting-JBNP of the Present Disclosure to Deliver an Agent to the Pancreas

Pancreas-targeting-JBNP-mRNA Cy5 is intravenously administered to BALB/cJ mice. Biodistribution of Pancreas-targeting-JBNP-mRNA Cy5 is monitored via IVIS® imaging. Then, the transfected pancreas is homogenized. Western blot and RT-qPCR are used to determine the transfection efficiency.

### Example 30: Examining the Ability of Intestine-Targeting-JBNP of the Present Disclosure to Deliver an Agent to the Intestine

Intestine-targeting-JBNP-mRNA Cy5 is intravenously administered to BALB/cJ mice. Biodistribution of Intestine-targeting-JBNP-mRNA Cy5 is monitored via IVIS® imaging. Then, the transfected heart is homogenized. Western blot and RT-qPCR are used to determine the transfection efficiency.

### Example 31: Examining the Ability of Tumor-Targeting-JBNP of the Present Disclosure to Deliver an Agent to a Solid Tumor

Tumor-targeting-JBNP-mRNA Cy5 is intravenously administered to xenograft model. Biodistribution of the Tumor-targeting-JBNP-mRNA Cy5 is monitored via IVIS® imaging. Then, the transfected solid tumor is homogenized. Western blot and RT-qPCR are used to determine the transfection efficiency. The mouse/mice are monitored for tumor size, weight, complete blood count (CBC), tumor necrosis factor-alpha (TNF-alpha) levels, interferon-gamma (IFN-gamma) levels, immunoglobulin G (IgG) levels, immunoglobulin M (IgM) levels, and toxicity.

### Example 32: Optimizing Dosage and Delivery of JBNP-Crispr

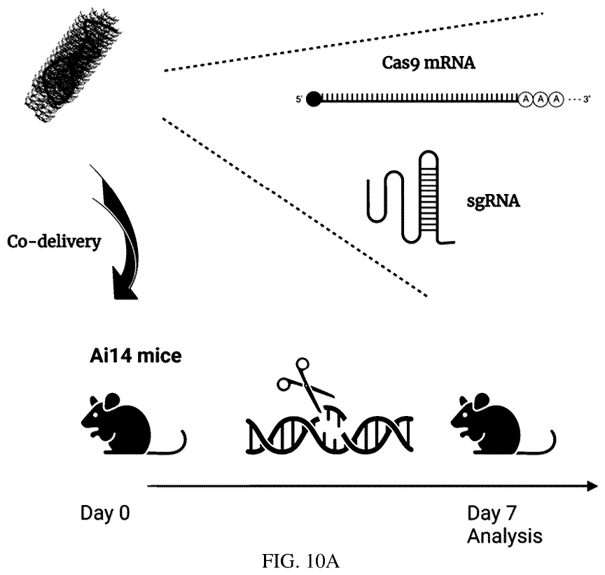

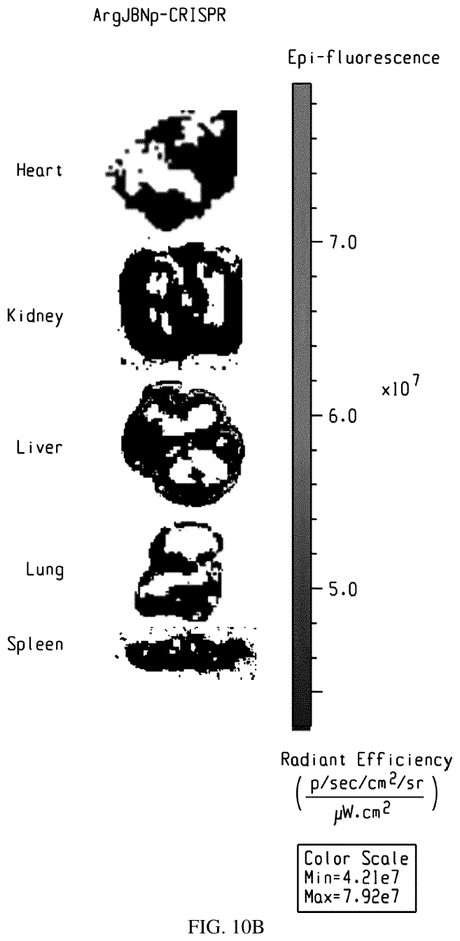

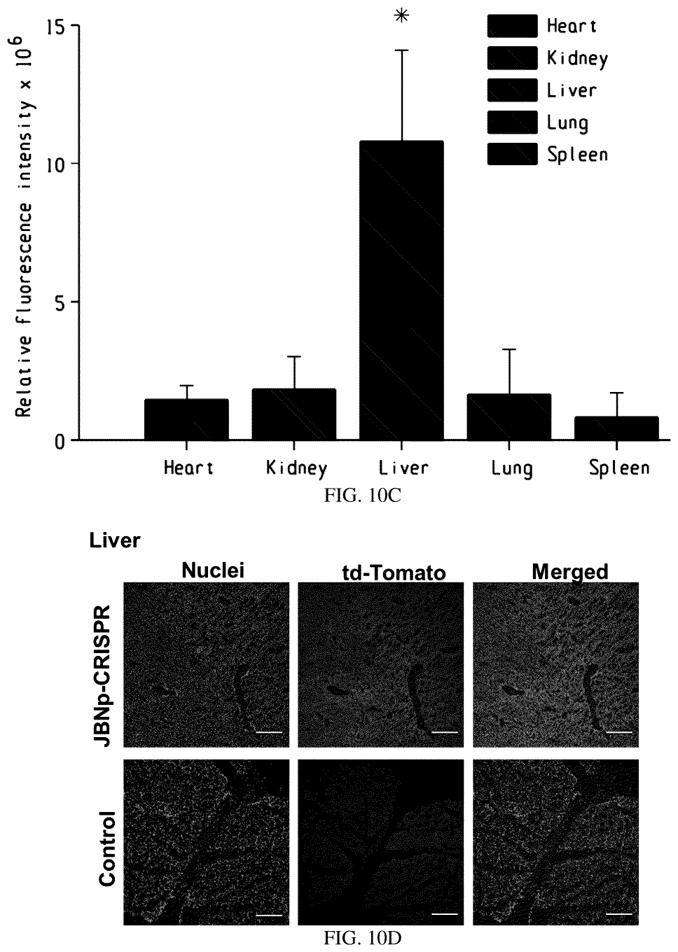

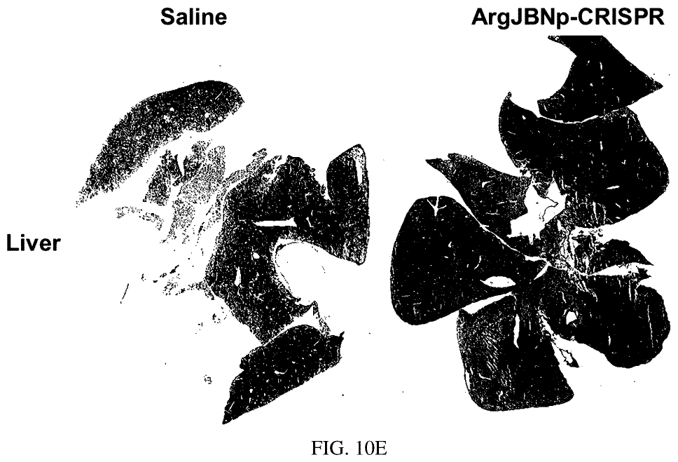

Optimized dosage and optimized delivery time for ArgJBNP-CRISPR was determined by performing gene editing with the Ail4 mouse model. ArgJBNP-CRISPR that is composed of Cas9mRNA (0.25 mg/kg) and sgLOXp was intravenously injected via tail vein injection or retro-orbital injection for 2-7 days. Positive tandem dimer Tomato (tdTomato) signals was monitored/detected via IVIS® imaging (FIGS. 10A-10D). Next-Generation Sequencing (NGS) can be used to quantify gene editing efficiency in the target organ.

### Example 33: Examining the Ability of JBNP-Crispr to Target and Edit the DNA of Major Organs

Assembled JBNP-CRISPR composed of Cas9mRNA and sgRB1 is intravenously injected to BALB/cJ mice for 7-28 days. Sanger sequencing or Next Generation Sequencing (NGS) is used to explore the RB1 gene editing efficiency.

### Example 34: Examining the Ability of JBNP-Crispr to Target and Edit the DNA of Different Organs Via the Attachment of a Targeting Molecule or Moiety

JBNP-CRISPR with a kidney targeting molecule or moiety can target the kidney and edit the DNA of kidney cells. JBNP-CRISPR with a heart targeting molecule or moiety can target the heart and edit the DNA of heart cells. JBNP-CRISPR with a spleen targeting molecule or moiety can target the spleen and edit the DNA of spleen cells. JBNP-CRISPR with lymph nodes targeting molecule or moiety can target the lymph nodes and edit the DNA of lymph node cells. JBNP-CRISPR with a lung targeting molecule or moiety can target the lungs and edit the DNA of lung cells. JBNP-CRISPR with a muscle targeting molecule or moiety can target muscles and edit the DNA of muscle cells. JBNP-CRISPR with a pancreas targeting molecule or moiety can target the pancreas and edit the DNA of pancreatic cells. JBNP-CRISPR with an intestine targeting molecule or moiety can target the intestines and edit the DNA of intestine cells.

### Example 35: Examining the Ability of JBNP-Crispr to Target and Edit the DNA of Solid-Tumors Via the Attachment of a Targeting Molecule or Moiety

JBNP-CRISPR with a tumor targeting molecule or moiety can target a solid-tumor and edit the DNA of tumor cells.

### Example 36: JBNP of the Present Disclosure do not Cause any Acute Toxicity

Acute toxicity was examined by tail-vein injection of the JBNP dosage (dosage optimized for each treatment) every 5 days for 20 days. H&E staining of histological sections of the liver, spleen, kidney, heart, pancreas, lung, brain, bone, and muscle were performed. No acute toxicity was detected in the examined organs from the administration of JBNP (FIGS. 11E-11H).

### Example 37: Examining Whether the JBNP of the Present Disclosure Causes an Innate Immune Response when Administered

JBNP is administered intravenously to BALB/cJ mice every 3 days. Serum (30 μl) is collected from submandibular laceration for each injection time point. IFN-gamma, IL-1beta, IL-6, IL-10, IL-12(p70), TNF-alpha, MCP-1, MIP-2, MIG, IL-2, IL-5, and IL-17 are examined via Immunology Multiplex Assay and enzyme-linked immunosorbent assay (ELISA).

### Example 38: JBNP of the Present Disclosure do not Cause an Adaptive Immune Response when Administered

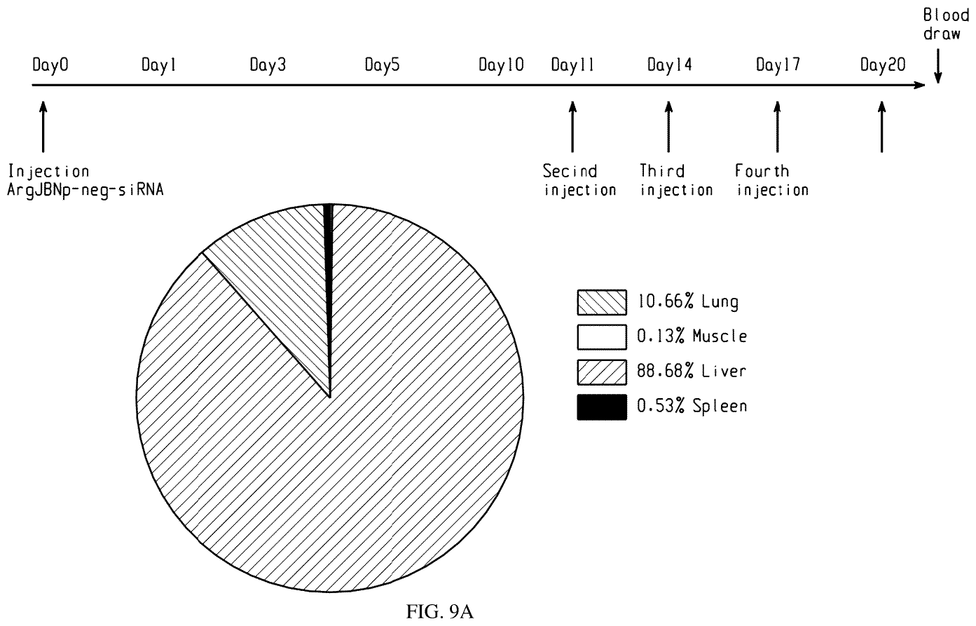

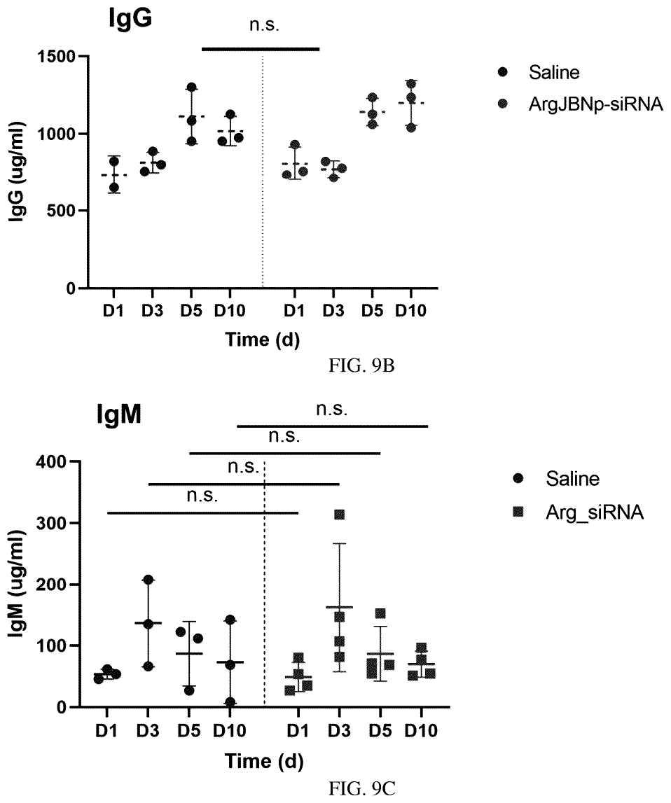

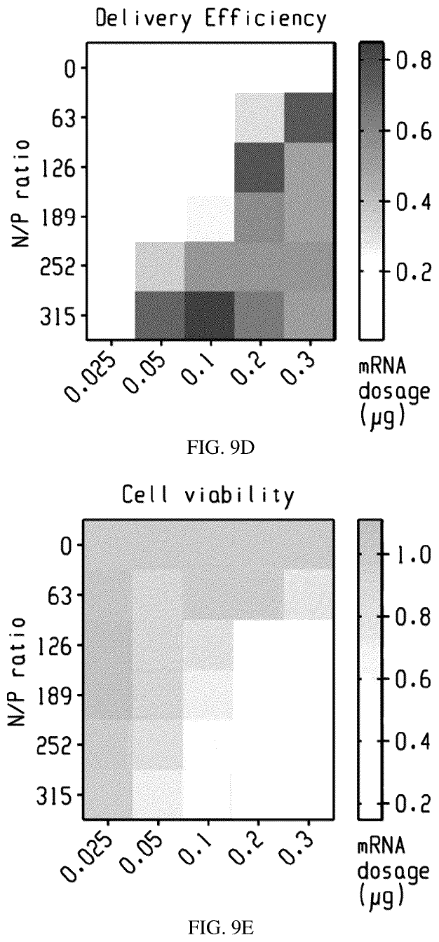

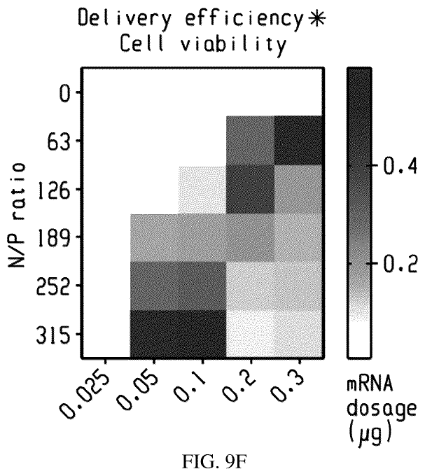

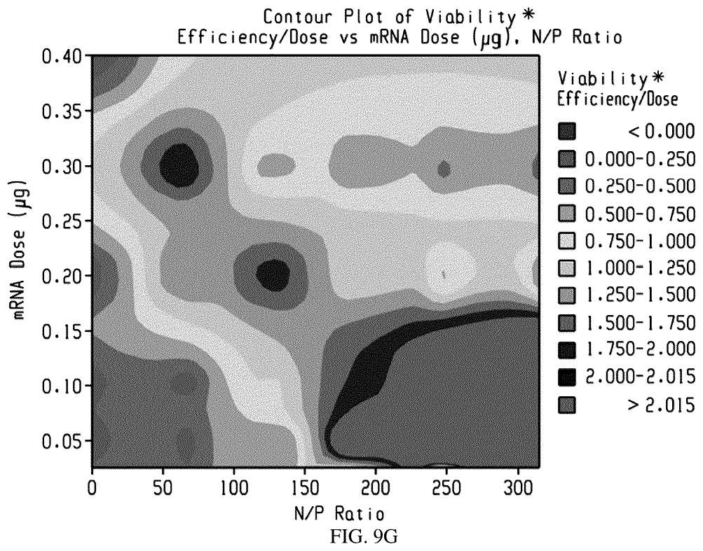

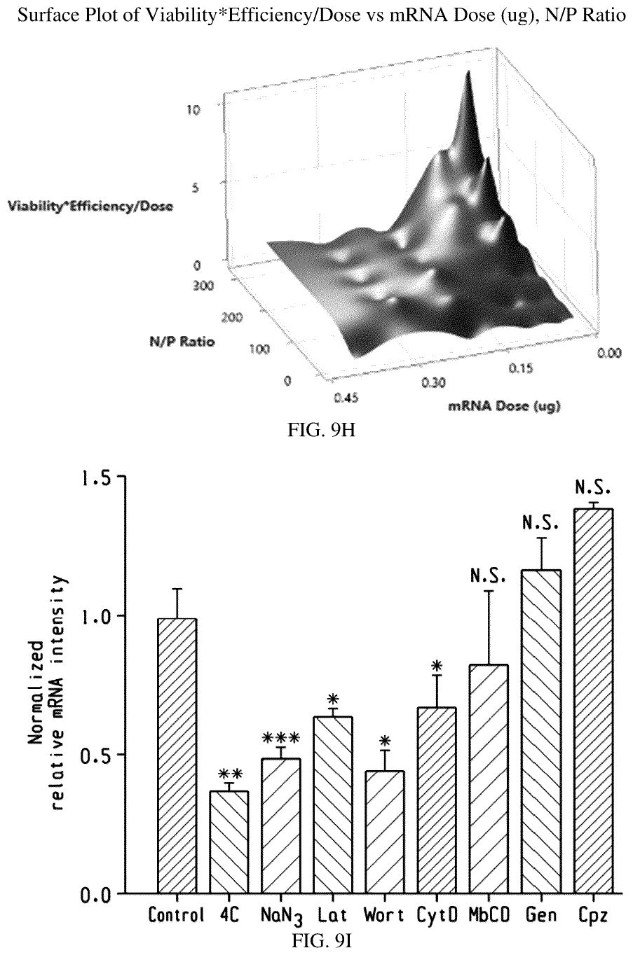

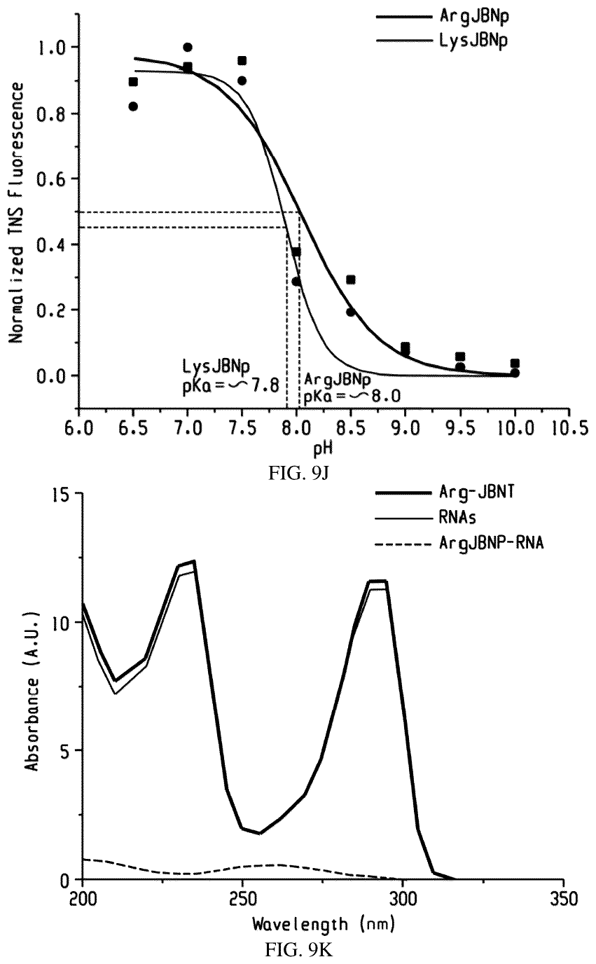

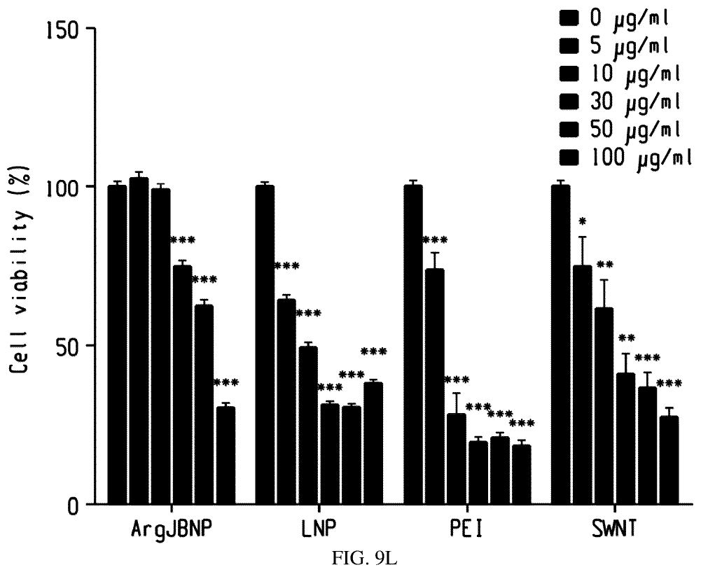

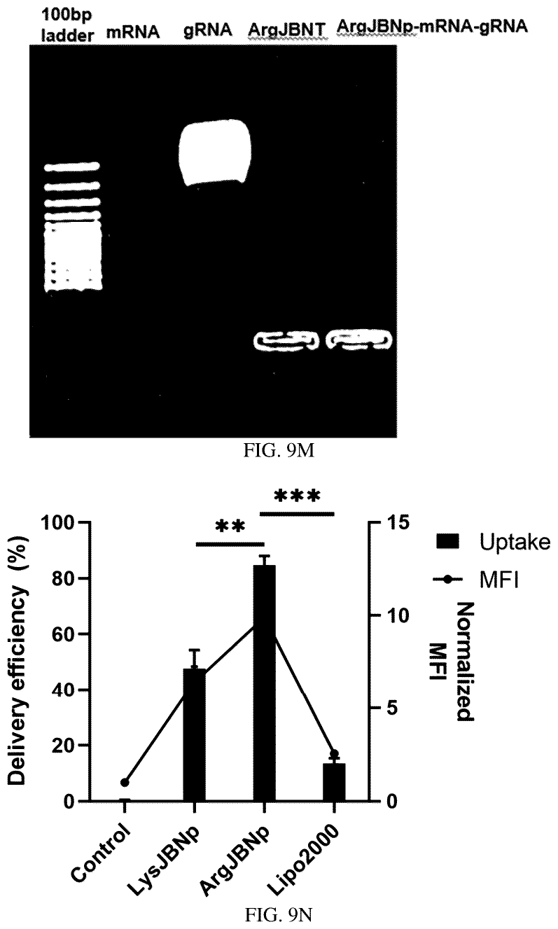

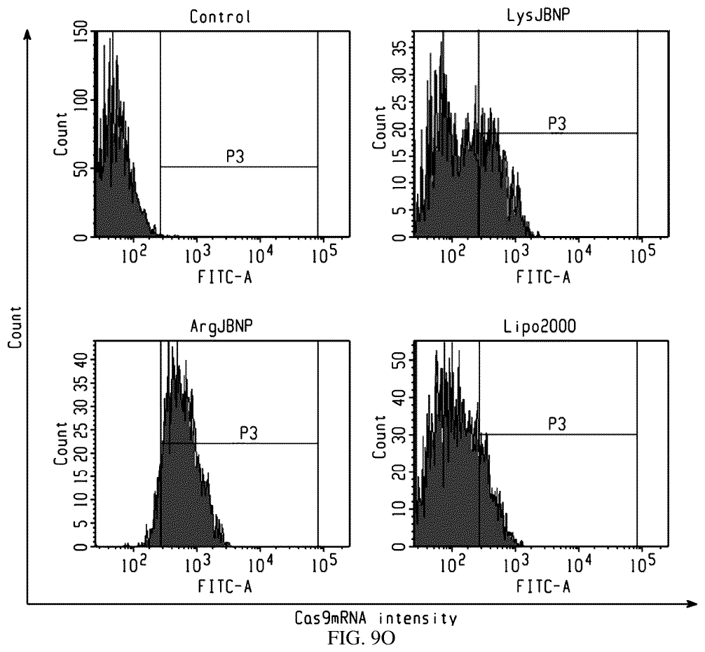

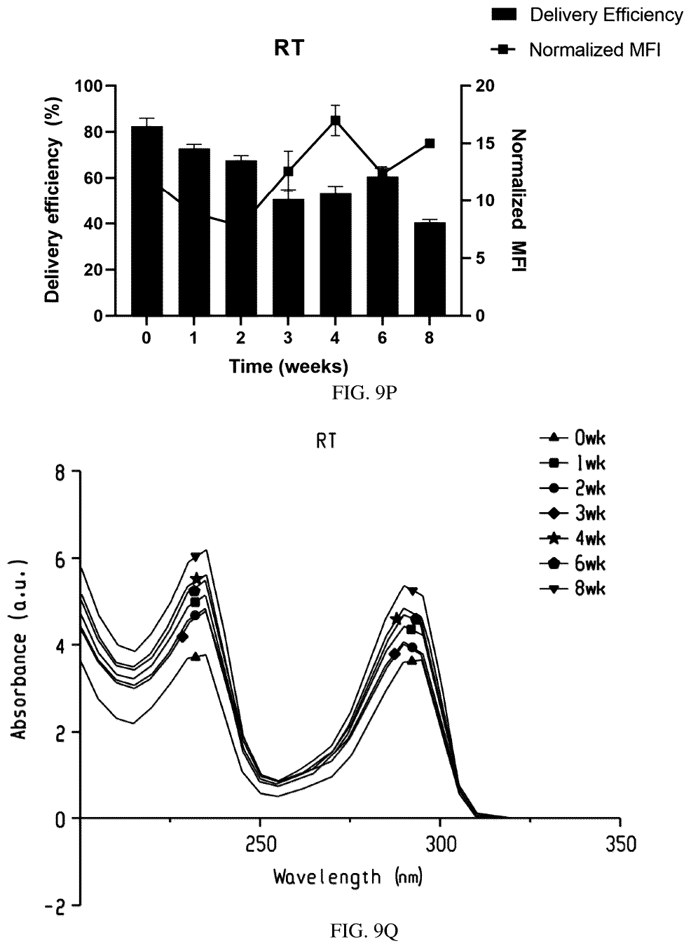

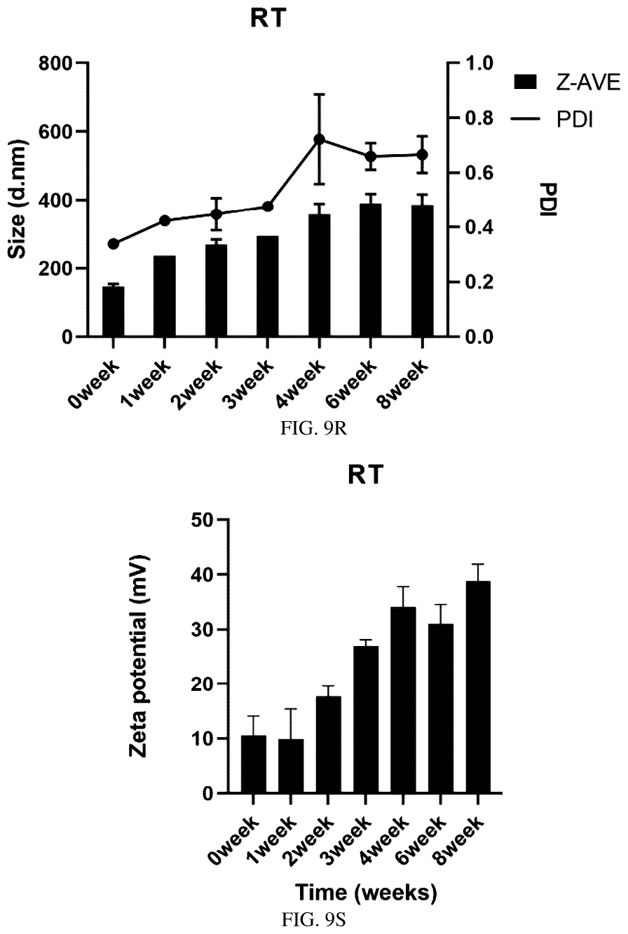

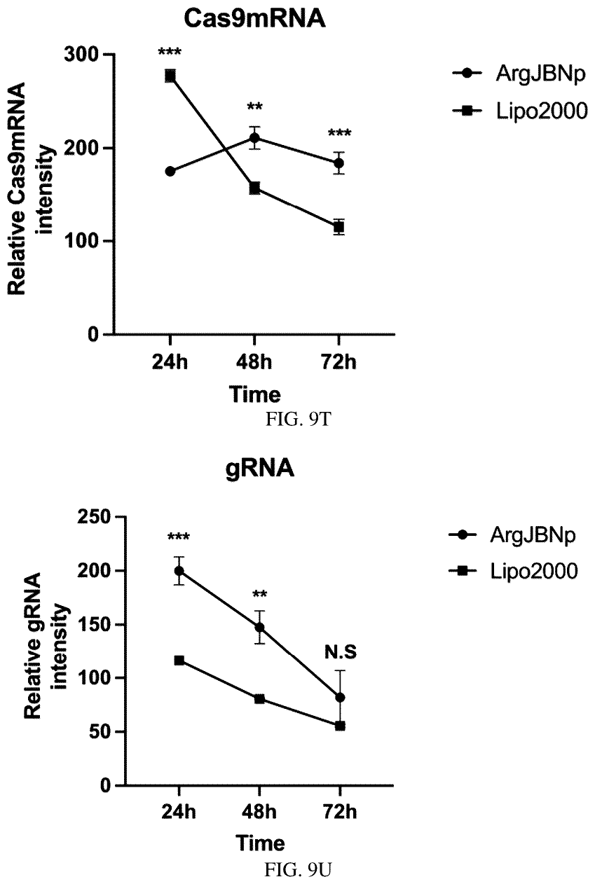

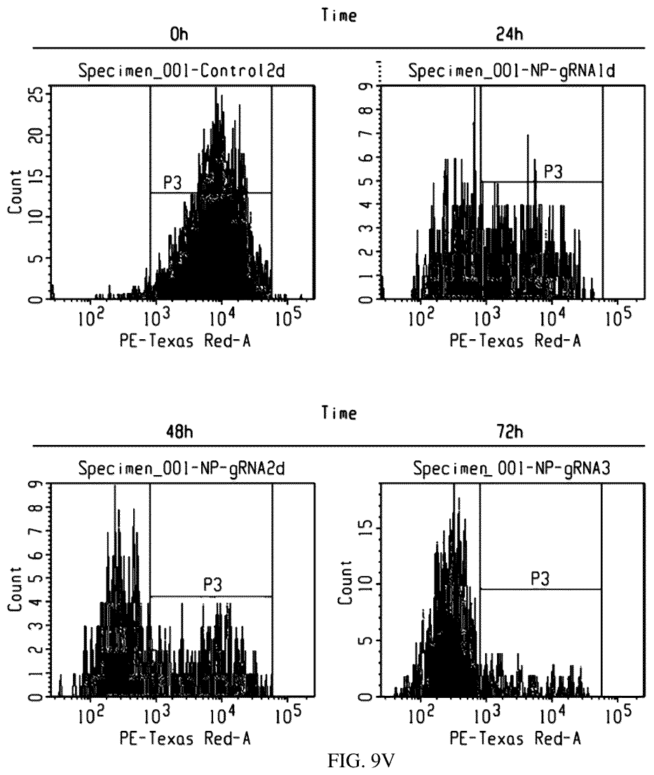

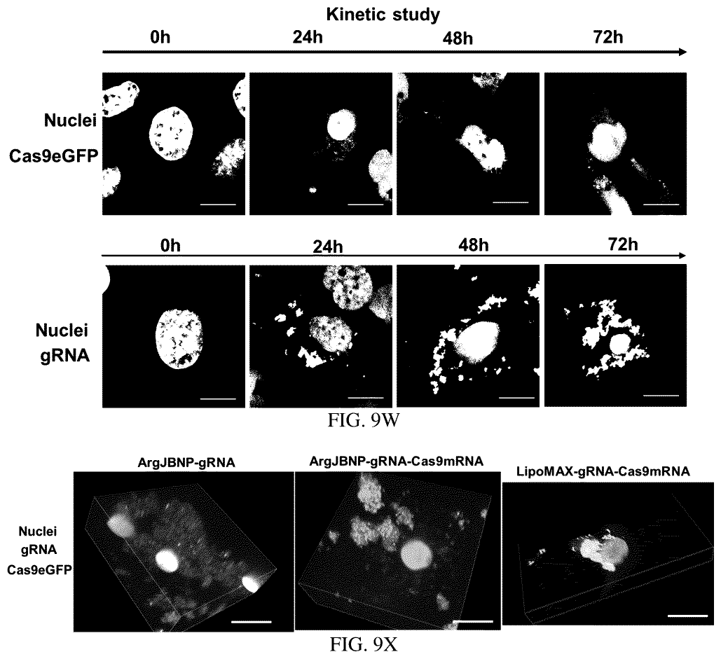

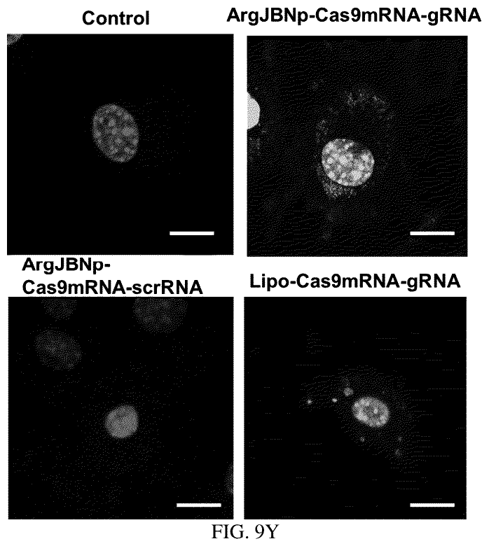

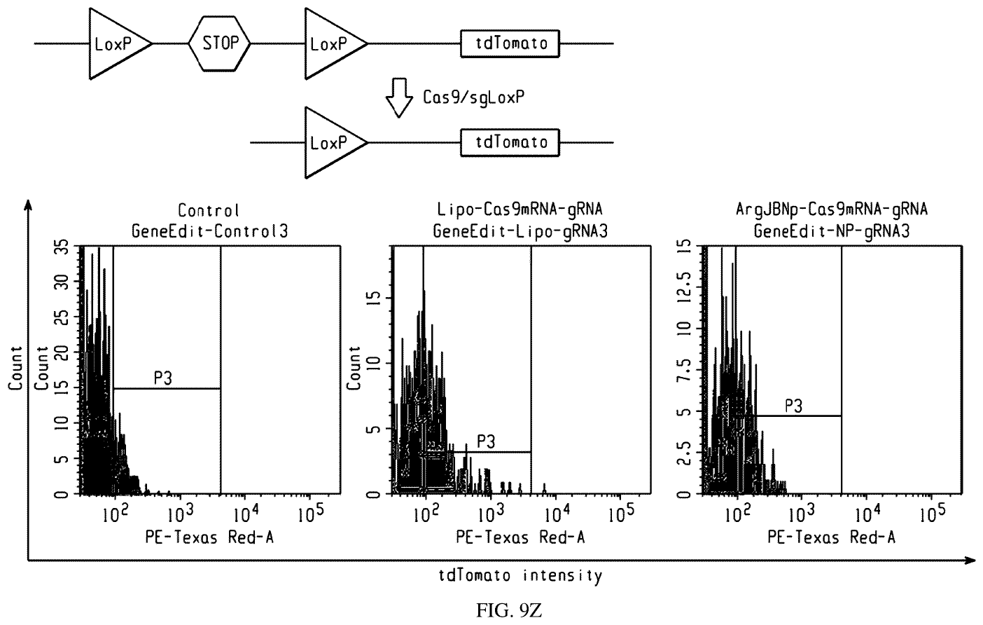

Adaptive immunity was not observed via IgG and IgM. JBNP was administered intravenously to BALB/cJ mice every 3 days, for 9 days. Serum was collected prior to receiving JBNP and after 10 day. Then, IgM and IgG levels were examined via ELISA (FIG. 9B, FIG. 9C, and FIG. 11F). Serum IgM and IgG antibody levels on day 10 were compared to the pre-injection baseline. An adaptive immune response was not observed after repeated administration of JBNP of the present disclosure (FIG. 9B, FIG. 9C, and FIG. 11F).

### Example 39: Examining Cellular Uptake Mechanism of the JBNP Based Upon the Modification or Composition of the JBNT

Cells were pretreated with several endocytic inhibitors, including macropinocytosis inhibitors (Latrunculin A, cytochalasin D), clathrin-mediated inhibitor (chlorpromazine), and a caveolae-mediated inhibitor (methyl-b-cyclodextrin). Then, the assembled ArgJBNP-mRNA AF488 were transfected into cells. After a 24-hour incubation, FACS was used to quantify the AF488 signal and to determine the uptake mechanism (FIG. 9I). Different side-chains likely have different cellular uptake mechanisms. For example, cellular uptake of JBNP comprising lysine-based JBNTs may be through macropinocytosis. Furthermore, cellular update of JBNP comprising arginine-based JBNT may be through clathrin-mediated endocytosis.

### Example 40: Endosomal Escape Mechanism of JBNPS Depends Upon the Modification or Composition of the JBNT

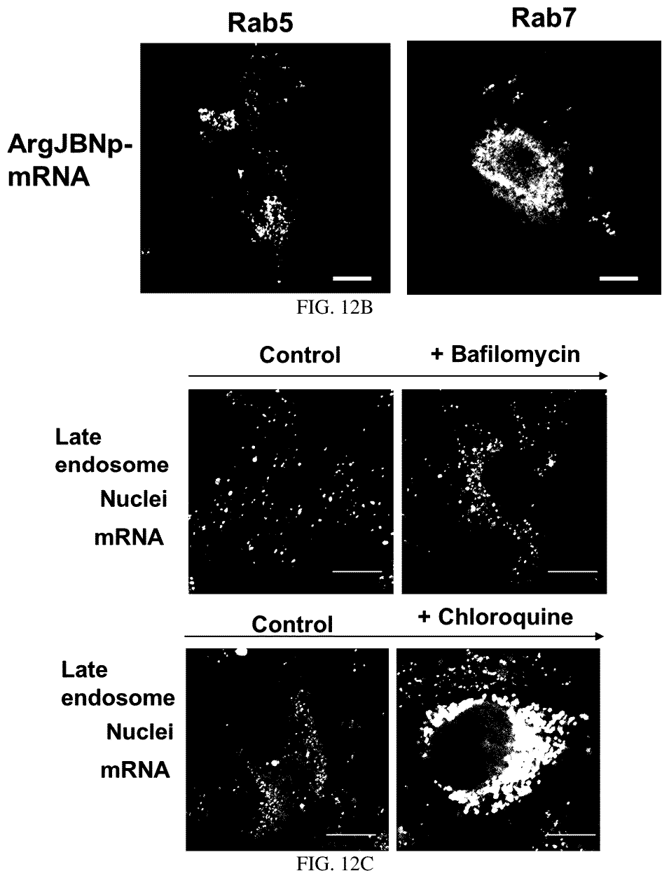

Endosomal escape was examined for JBNPs comprising different types of JBNT of the present disclosure. The endosomes were examined by staining the cells contacted with the tested JBNP with LysoTracker™ RED which stains for and visualized endosomes in cells. The degree of colocalization was quantified based on Pearson's correlation coefficient (r) using the Image J software following the colocalization threshold and coloc2 plugin. Bafilomycin A1 and chloroquine inhibitors were used in a pre-treatment to demonstrate the proton-sponge effect. Calcein assay and modeling were used to explore the pore-formation to escape the endosomes. JBNP comprising lysine-based JBNTs has significantly better endosomal escape, which appears to be due to the proton sponge effect (U.S. Patent Application Publication No. 20220133893 A1). Furthermore, JBNP comprising arginine-based JBNTs have significantly enhanced endosomal escape, which appears to be due to pore-forming effect to escape early endosome (FIG. 12B and FIG. 12C).

### Example 41: JBNP of the Present Disclosure are Highly Biocompatible with Low Cell-Toxicity

Human chondrocytes were seeded (5000 cells/well) and incubated overnight. Then, cell viability of various vectors, including JBNP, were determined by Cell Counting Kit-8 assay (Sigma). After coincubation for 24 hours, the absorbance was obtained by a microplate reader. The JBNP of the present disclosure are highly biocompatible with a lower cell-toxicity than lipid nanoparticles, polymer nanoparticles, such as polyethylenimine nanoparticles (PEI), lipid nanoparticles (LNP), and single-wall carbon nanotubes (SWNT) (FIG. 9L).

### Example 42: JBNP of the Present Disclosure can be Utilized for CRISPR Delivery

C28/I2 cells were seeded in a 24-well plate (5000 cells/well) and incubated overnight. Co-assembled JBNP-Cas9mRNA-eGFP and gRNA-ATTO550 dye were directly transferred to the well and transfected for 72 hours. Then, FACS was performed to quantify the co-delivery in a cell. (FIG. 9T and FIG. 9U) Co-delivery of Cas9mRNA-eGFP and gRNA-ATTO550 were imaged by confocal laser scanning laser microscope (FIG. 9W).

### Results for Examples 10-42

The exemplary Transferrin-targeting-JBNP delivering mRNA to the C28/I2 cells is shown in FIG. 7E. The exemplary targeting in vivo study is also shown in FIG. 7B and FIG. 7C. Cartilage targeting WYRGRL-ArgJBNP increased the retention time in the knee joint, delivering siRNA.

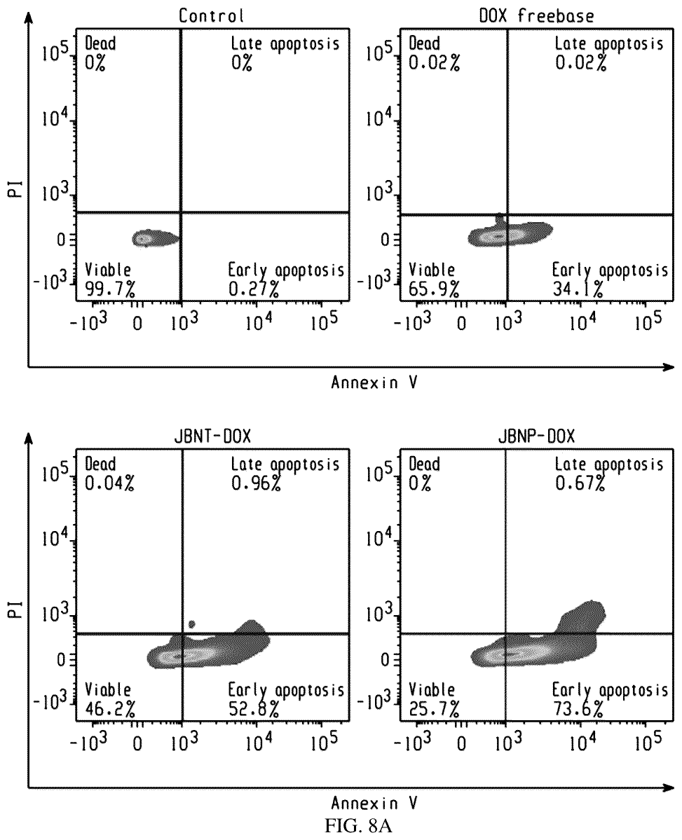

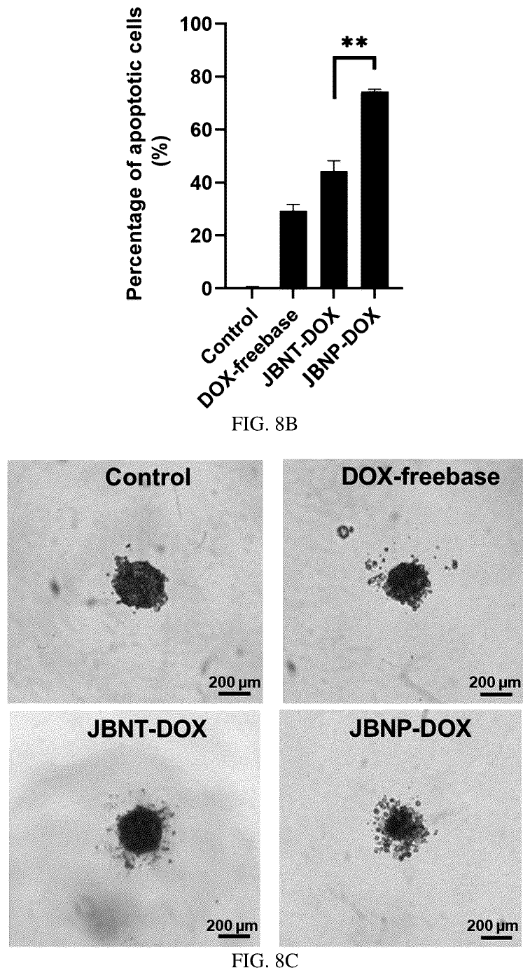

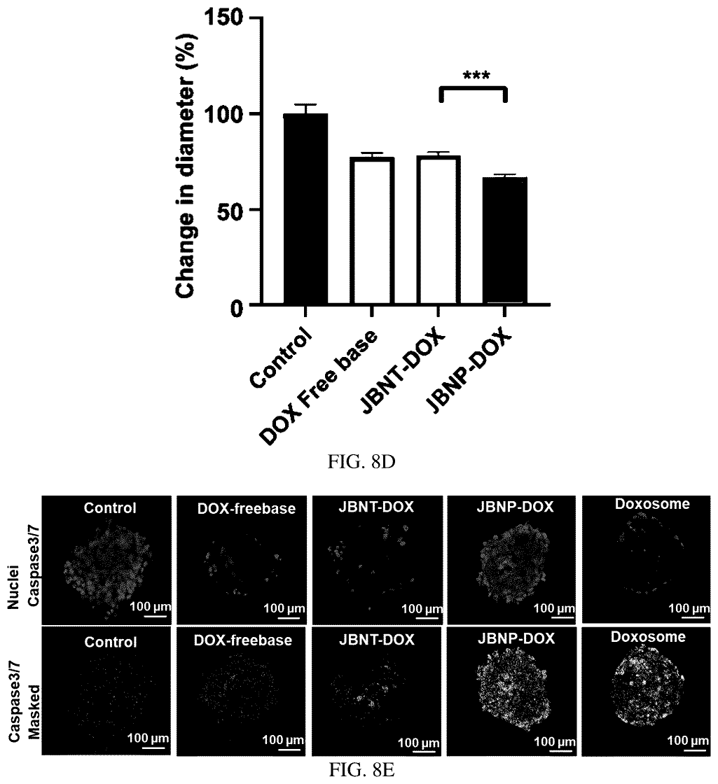

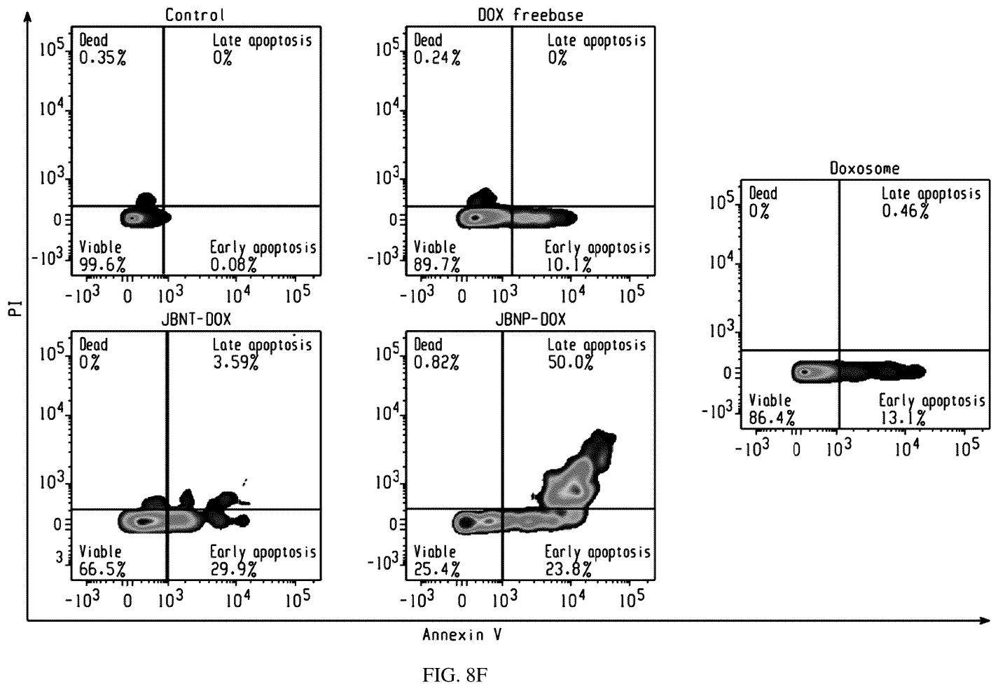

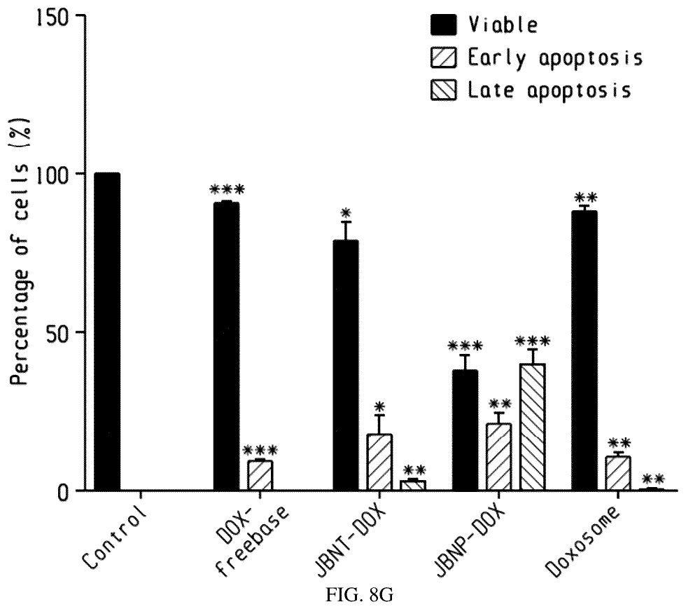

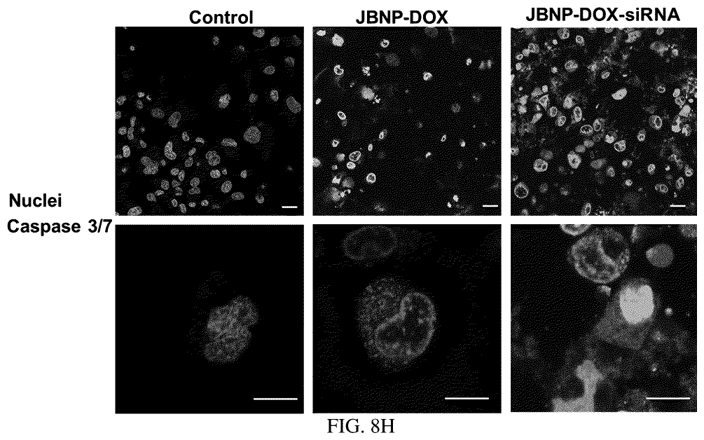

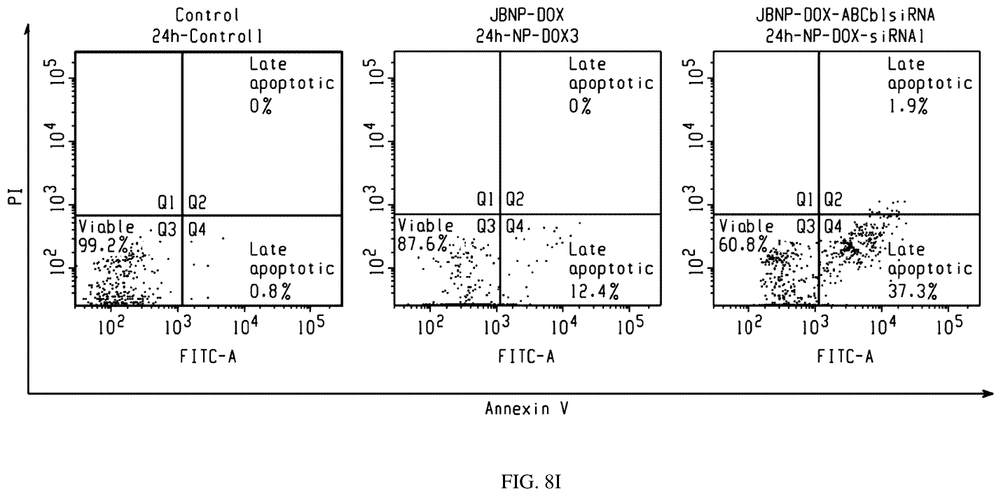

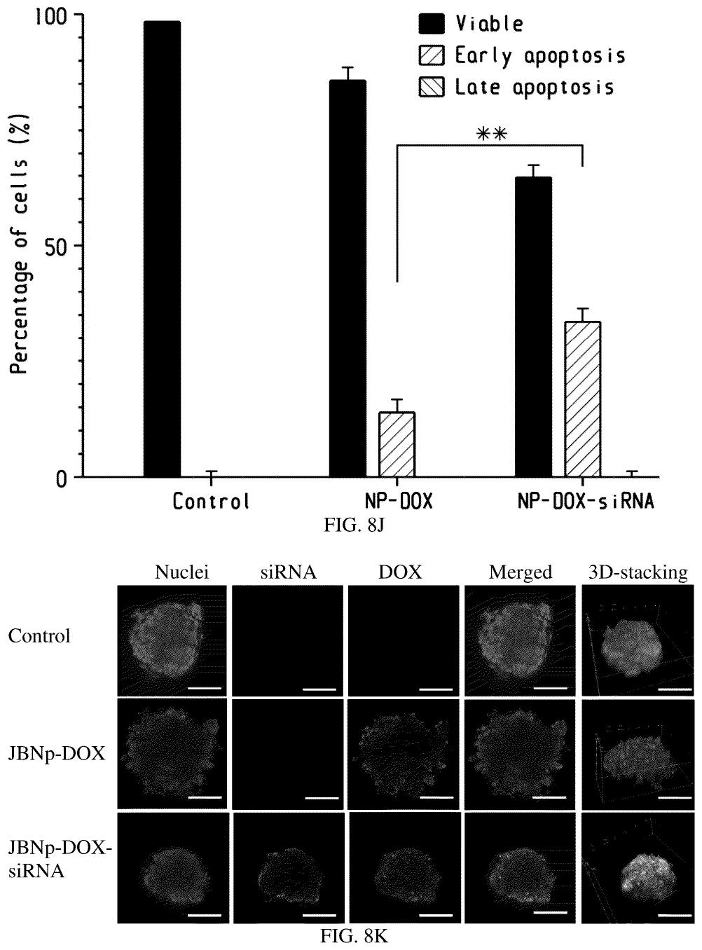

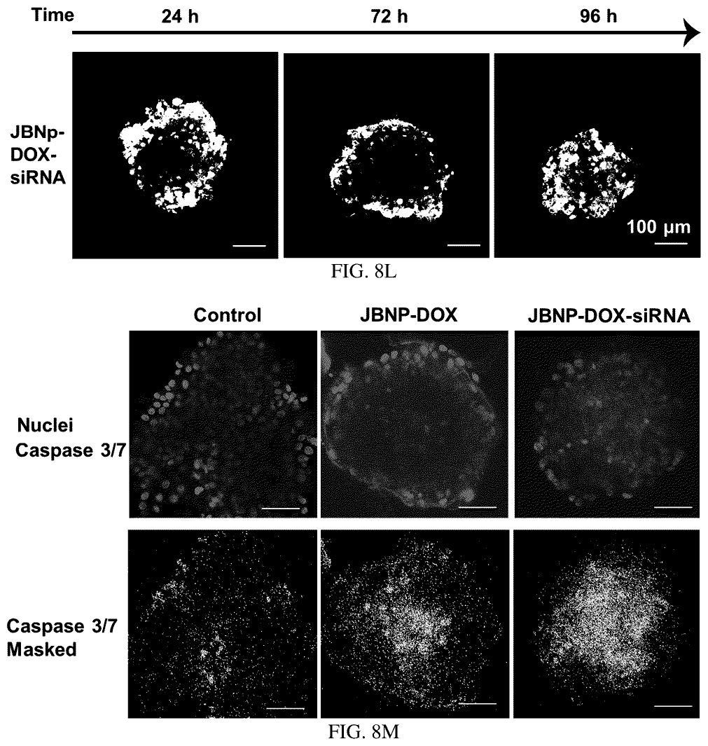

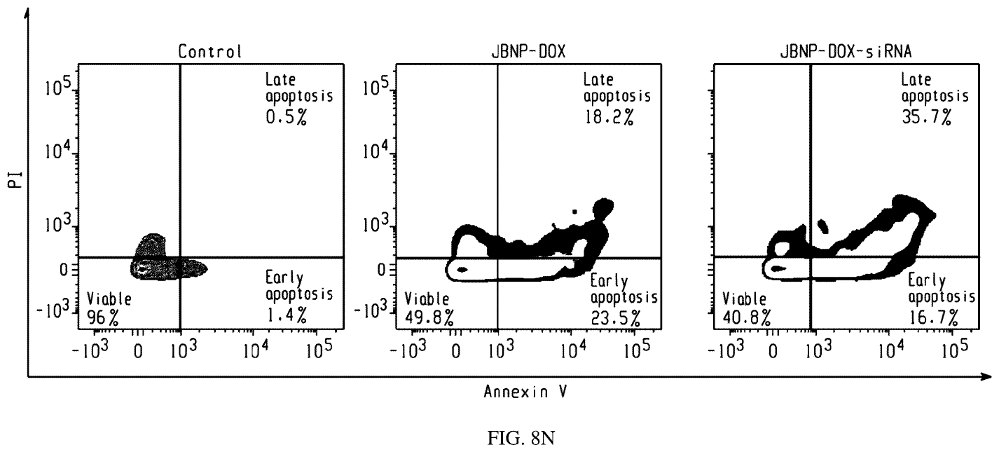

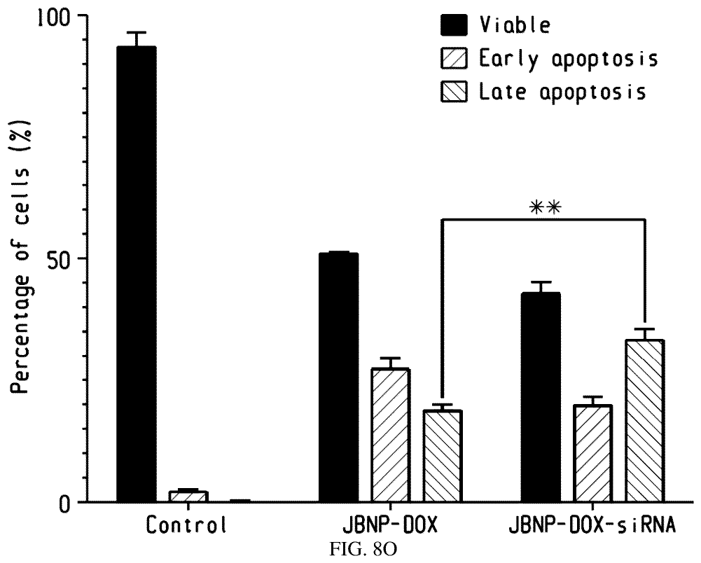

FIGS. 8A-8G show a small molecule drug (doxorubicin) can be delivered via JBNP. FIGS. 8A and 8B show a monolayer of transfected cells examined for apoptosis markers. FIGS. 8C and 8D demonstrate the inhibition of spheroid formation when treated with JBNP-DOX. FIGS. 8E-8G demonstrate the delivery of JBNP-DOX to ovarian cancer spheroids. FIGS. 8H-8J demonstrate that small molecule drugs (doxorubicin) can be co-delivered with siRNA via JBNP into a monolayer of SKOV-3 cells. FIGS. 8K-8O demonstrate the co-delivery of DOX and siRNA to SKOV-3 ovarian cancer spheroids. FIGS. 8K and 8L are fluorescence images of DOX and siRNA-AF488 delivered via JBNP. FIG. 8M has representative images of spheroids stained with apoptosis markers Caspase 3/7. FIGS. 8N and 8O show the FACS plots and quantification of the apoptosis analysis.

FIG. 9A shows the biodistribution of siRNAs delivered via ArgJBNP in BALB/Cj mice. FIGS. 9B and 9C show the immunogenicity study of the ArgJBNP-siRNA. FIGS. 9D-9F is the optimization of ArgJBNP-mRNA formulation based on the delivery efficiency and cell viability. FIGS. 9G and 9H is the response surface methodology (RSM) utilized to optimize the ArgJBNP-mRNA formulation. FIG. 9I is an uptake mechanism analysis of ArgJBNP-mRNA. FIG. 9J is the 6-(β-Toluidino)-2-naphthalene-6-sulfonic acid (TNS) assay to analyze the pKA of the ArgJBNP-mRNA. FIG. 9K is the UV-VIS analysis. FIG. 9L shows the results for the cell viability assay performed of ArgJBNP. FIG. 9M shows the gel retardation assay of ArgJBNP-mRNA-gRNA. FIGS. 9N and 9O show the flow cytometry analysis that examines the delivery efficiency of ArgJBNP-mRNA comparing the LysJBNP-mRNA and Lipofectamine™ 2000-mRNA. FIGS. 9P-9S show the excellent stability of ArgJBNP-mRNA in room-temperature for 2 weeks. FIGS. 9T and 9U show the time-dependent delivery of Cas9mRNA and gRNA in C28/I2 cells. FIG. 9V show the time-dependent gene editing of the RFP-HUVEC cells analyzed by flow-cytometry. FIG. 9W is the kinetic study of Cas9eGFP mRNA and gRNA delivery via ArgJBNP. FIG. 9X is the 3D rendering image of delivery of gRNA and Cas9mRNA into C28/I2 cell. In FIGS. 9Y and 9Z, ArgJBNP was able to deliver Cas9mRNA and gRNA to and edit chondrocytes of Ail4 mice. Additionally, FIGS. 10A-10D show the ability of the ArgJBNP-CRISPR to edit in vivo. FIG. 10E show a representative image of an H&E stained section of a liver after injection of ArgJBNP-CRISPR.

Moreover, FIG. 11A and FIG. 11B show the biodistribution of mCherry mRNA delivered via ArgJBNP. FIGS. 11C and 11D show the protein corona analysis of the ArgJBNP-mRNA. FIG. 11C demonstrates the protein corona via SDS-PAGE. FIG. 11D is the LC/MS/MS analysis of the protein corona of ArgJBNP-mRNA.

Additionally, FIGS. 11E-11H examined the immunogenicity of ArgJBNP-mRNA. FIG. 11E shows H&E stained sections of organs after repeated injection of ArgJBNP-scrambled mRNA. FIG. 11F shows the IgG and IgM immune response. FIG. 11G is the CBC performed, including WBC, RBC, HGB, and PLT counts. FIG. 11H shows the change of body weight.

FIG. 12A shows the UV-VIS analysis on pH 5.2 and pH 7.4 of ArgJBNP. FIG. 12B shows the endosomal escape of ArgJBNP at early endosome (Rab5) and late endosome (Rab7). Moreover, FIG. 12C demonstrate the endosomal escape of ArgJBNP via the use of inhibitors.

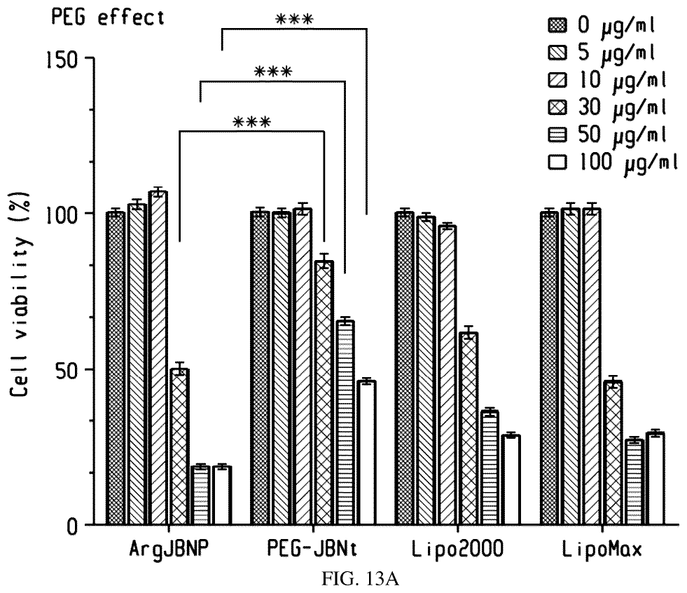

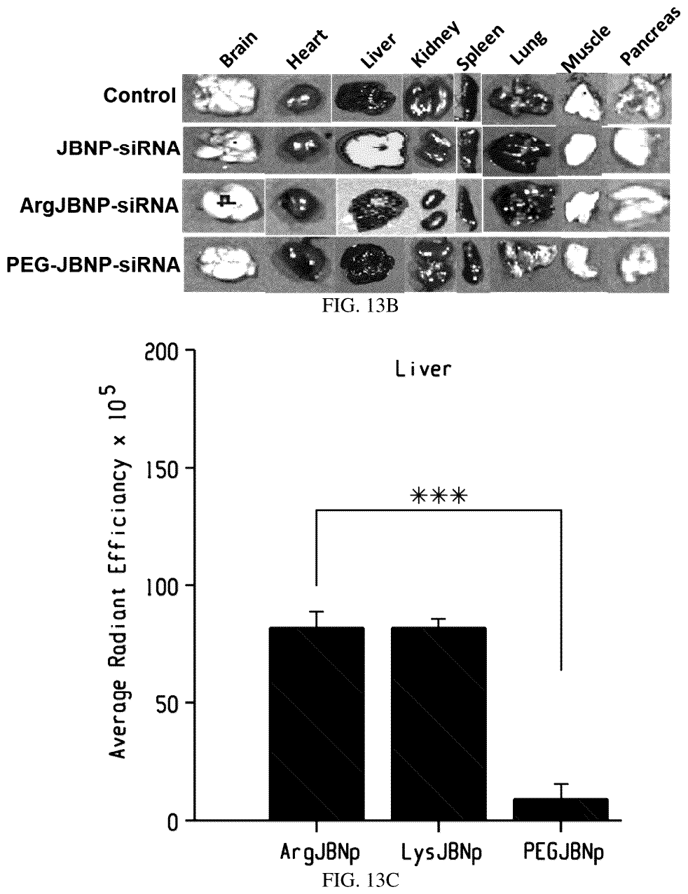

Moreover, FIG. 13A shows the cell viability assay performed for PEG-JBNT. PEGylation protects from non-specific cellular uptake (such as immune cells and other undesired cells) and a targeting peptide can be used with the PEG, so that active targeting can be achieved. FIG. 13B and FIG. 13C show the biodistribution of PEG-JBNP delivering siRNA to BALB/cJ mice.

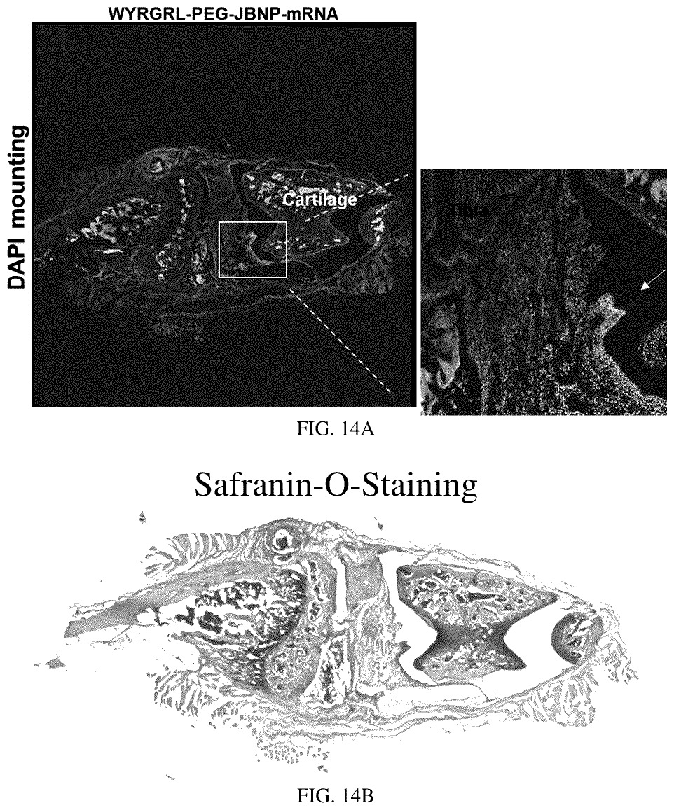

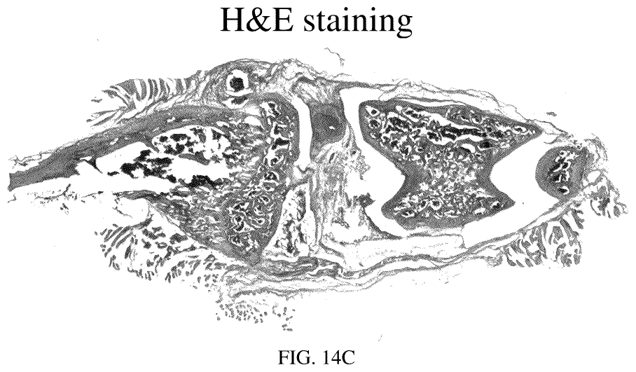

The exemplary FIGS. 14A-14C demonstrates the combined PEG effect and targeting ability of WYRGRL-PEG-JBNP-mRNA-cy5.

The compositions, methods, and articles can alternatively comprise, consist of, or consist essentially of, any appropriate materials, steps, or components herein disclosed. The compositions, methods, and articles can additionally, or alternatively, be formulated so as to be devoid, or substantially free, of any materials (or species), steps, or components, that are otherwise not necessary to the achievement of the function or objectives of the compositions, methods, and articles.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A self-assembled nanomaterial comprising a Janus base nanotube, wherein the Janus base nanotube comprises at least one compound represented by Formulas I to XII, or a pharmaceutically acceptable salt thereof: 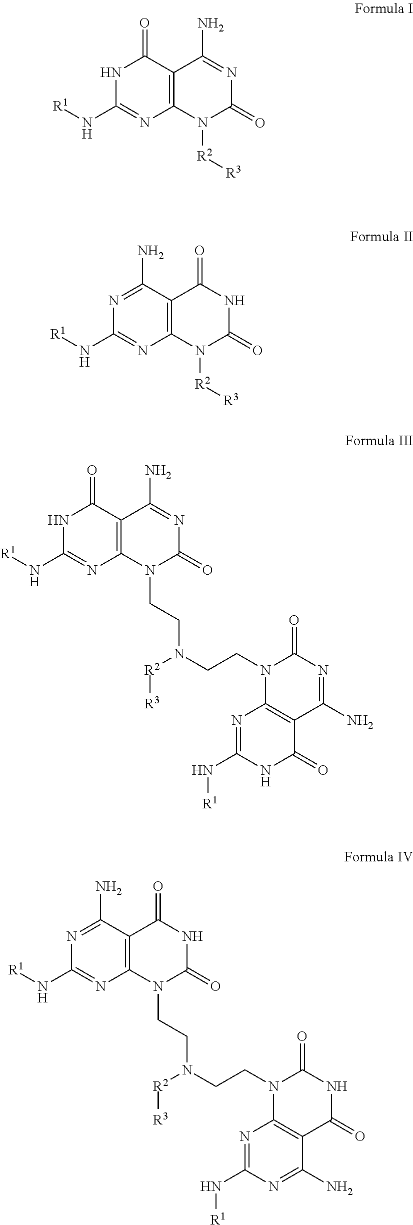 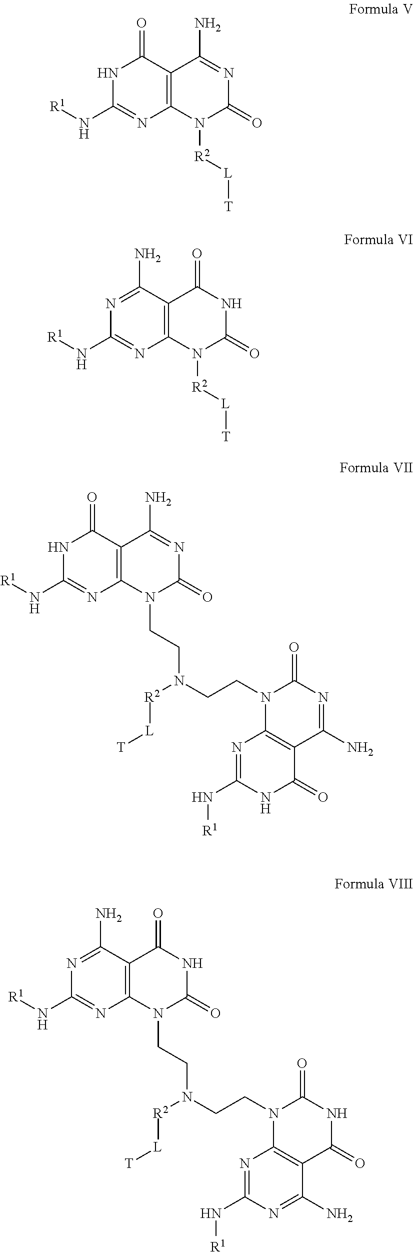 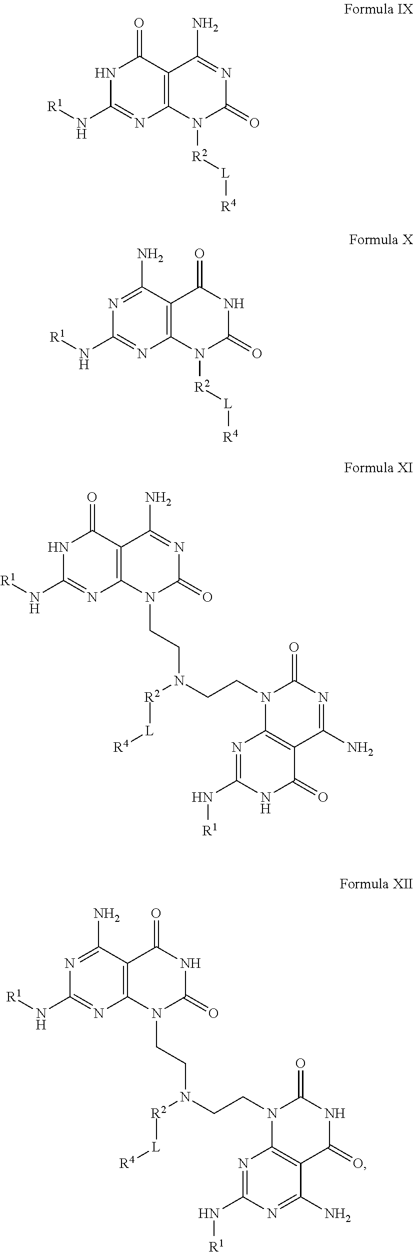
wherein,
R¹ is H or CH₃;
R² is (CH₂)ⱼ, (CH₂CH₂O)ₖ, or (CH₂CH₂NH)ₘ, wherein j, k, and m are independently an integer from 1 to 200;
R³ is an α-amino acid, a β-amino acid, an α-polypeptide, or a β-polypeptide;
L is a bond or a linker group;
T is a biologically active molecule or a targeting molecule; and
R⁴ is a coating material.

2. The self-assembled nanomaterial of claim 1, wherein the Janus base nanotube comprises at least one compound represented by Formulas I to IV.

3. The self-assembled nanomaterial of claim 1, wherein the Janus base nanotube comprises at least one compound represented by Formulas V to VIII.

4. The self-assembled nanomaterial of claim 1, wherein the Janus base nanotube comprises at least one compound represented by Formulas IX to XIII.

5. The self-assembled nanomaterial of claim 1, wherein the Janus base nanotube comprises a combination of:
a compound represented by Formulas I to IV and a compound represented by Formulas V to VIII;
a compound represented by Formulas I to IV and a compound represented by Formulas IX to XIII;
a compound represented by Formulas V to VIII and a compound represented by Formulas IX to XIII;
a compound represented by Formulas I to IV, a compound represented by Formulas V to VIII, and a compound represented by Formulas IX to XIII; or
a combination thereof.

6. The self-assembled nanomaterial of claim 1, wherein L is the linker group and is selected from an acid-cleavable group, a reducible disulfide group, an α-amino acid, a β-amino acid, an α-polypeptide, a β-polypeptide, an enzyme cleavable group, and a stimuli-responsive group.

7. The self-assembled nanomaterial of claim 6, wherein
the acid-cleavable group is N-acyl hydrazone, a carbonate group, or an ester group;
the reducible disulfide linker is N-succinimidyl-4-(2-pyridyldithio)pentanoate (SPP), N-succinimidyl-4-(2-pyridyldithio)butyrate (SPDB), or 4-(4′-acetylphenoxy)butanoic acid (AcBut), Val-Cit dipeptide, Phe-Lys dipeptide, an α-methyl substituted disulfide, an engineered cysteine residue, or a thiol-containing maytansinoid;
the stimuli-responsive linker is a trans-cyclooctene linker or a thioether-containing linker; or
the enzyme cleavable linker is GPLGOAGQ (SEQ ID NO:89), GDEVEAPKGC (SEQ ID NO:90), citrulline-valine, a glycosidase-cleavable linker, a β-glucoronidase-cleavable linker, a β-Galactosidase-cleavable linker, a phosphatase-cleavable linker, a pyrophosphate-containing linker, a dipeptide-containing linker, Gly-Phe-Leu-Gly, Ala-Leu-Ala-Leu, Phe-Lys-PABC (para-aminobenzyl carbamate), a Val-Cit-PABC containing linker, a Glu-Val-Cit-containing linker, or a Val-Ala containing linker.

8. The self-assembled nanomaterial of claim 1, wherein R⁴ is the coating material and is selected from a polymer, a peptide, a polypeptide, a lipid-based material, phosphate ester, or a biomimetic membrane.

9. The self-assembled nanomaterial of claim 1, wherein R⁴ is selected from polyethylene glycol, chitosan, hyaluronic acid, a poloxamer, polyvinyl alcohol, a polysaccharide, a neutral poly(amino acid), a negatively charged poly(amino acid), phytochelatin, a self peptide, and an antithrombotic peptide.

10. The self-assembled nanomaterial of claim 1, wherein T is the targeting molecule and is selected from a biologically active molecule, an amphiphilic polymer, an aptamer, a peptide, a cyclic peptide, a protein, a polysaccharide, a polyunsaturated fatty acid, and a carbohydrate.

11. The self-assembled nanomaterial of claim 1, wherein T is the targeting molecule and the self-assembled nanomaterial further comprises a biologically active molecule covalently or non-covalently adhered to the self-assembled nanomaterial.

12. The self-assembled nanomaterial of claim 10, wherein the biologically active molecule is noncovalently adhered to the self-assembled nanomaterial.

13. The self-assembled nanomaterial of claim 10, wherein the biologically active molecule is at least partially encapsulated by the self-assembled nanomaterial.

14. The self-assembled nanomaterial of claim 1, wherein the biologically active molecule comprises a nucleic acid, a protein, a peptide, cyclic peptide, a small molecule drug, or a combination thereof.

15. The self-assembled nanomaterial of claim 1, wherein the biologically active molecule comprises miRNA, siRNA, mRNA, gRNA, crRNA, tracrRNA, tRNA, ssDNA, dsDNA, cDNA, or a combination thereof.

16. The self-assembled nanomaterial of claim 1, wherein the Janus base nanotube is present in an amount of 0.1 wt % to 99.9 wt % based on the total weight of the self-assembled nanomaterial.

17. The self-assembled nanomaterial of claim 1, wherein a concentration of the Janus base nanotube in the self-assembled nanomaterial is 1 μg/mL to 1 g/ml.

18. The self-assembled nanomaterial of claim 1, wherein pH of the self-assembled nanomaterial is from 1 to 10.

19. The self-assembled nanomaterial of claim 1, further comprising an extracellular matrix (ECM) molecule.

20. The composition of claim 18, wherein the ECM molecule comprises hydroxyapatite, fibronectin, Matn1, Matn3, laminin, cartilage oligomeric matrix protein, a collagen, elastin, vitronectin, fibrillin, perlecan, fibrinogen, osteonectin, tenascin, thrombospondin, an intercellular adhesion molecule (ICAM1-5), an integrin, a proteoglycan, a glycoprotein, or a combination thereof.

21. (canceled)


SEQUENCE LISTING (90 sequences)

SEQ ID NO: 1 - protein, 7 aa, synthetic construct
  MOD_RES 2: note = L-2-naphthylalanine
FXRRRRQ

SEQ ID NO: 2 - protein, 7 aa, synthetic construct
  MOD_RES 2: note = L-2-naphthylalanine
RRXFRRQ

SEQ ID NO: 3 - protein, 7 aa, synthetic construct
  MOD_RES 5: note = L-2-naphthylalanine
RRFRXRQ

SEQ ID NO: 4 - protein, 7 aa, synthetic construct
  MOD_RES 6: note = L-2-naphthylalanine
FRRRRXQ

SEQ ID NO: 5 - protein, 8 aa, synthetic construct
  MOD_RES 2: note = L-2-naphthylalanine
FXRRRRRQ

SEQ ID NO: 6 - protein, 6 aa, synthetic construct
  MOD_RES 2: note = L-2-naphthylalanine
RXRRRQ

SEQ ID NO: 7 - protein, 8 aa, synthetic construct
  MOD_RES 6: note = L-2-naphthylalanine
RFRFRXRQ

SEQ ID NO: 8 - protein, 8 aa, synthetic construct
  MOD_RES 3: note = L-2-naphthylalanine
FFXRRRRQ

SEQ ID NO: 9 - protein, 7 aa, synthetic construct
  MOD_RES 5: note = L-2-naphthylalanine
  MOD_RES 1: note = D-arginine
XRFRXRQ

SEQ ID NO: 10 - protein, 8 aa, synthetic construct
  MOD_RES 1: note = D-phenylalanine
  MOD_RES 4: note = D-arginine
  MOD_RES 6: note = D-arginine
  MOD_RES 2: note = L-2-naphthylalanine
XXRXRXRQ

SEQ ID NO: 11 - protein, 8 aa, synthetic construct
  MOD_RES 3: note = L-2-naphthylalanine
  MOD_RES 5: note = D-arginine
  MOD_RES 7: note = D-arginine
  MOD_RES 2: note = D-phenylalanine
FXXRXRXQ

SEQ ID NO: 12 - protein, 7 aa, synthetic construct
  MOD_RES 3: note = D-arginine
  MOD_RES 5: note = D-arginine
  MOD_RES 2: note = D-2-naphthylalanine
FXXRXRQ

SEQ ID NO: 13 - protein, 7 aa, synthetic construct
FWRRRRQ

SEQ ID NO: 14 - protein, 7 aa, synthetic construct
  MOD_RES 2: note = L-2-naphthylalanine
YXRRRRQ

SEQ ID NO: 15 - protein, 7 aa, synthetic construct
  MOD_RES 2: note = L-2-naphthylalanine
HXRRRRQ

SEQ ID NO: 16 - protein, 7 aa, synthetic construct
  MOD_RES 2: note = L-2-naphthylalanine
  MOD_RES 1: note = L-phenylglycine
XXRRRRQ

SEQ ID NO: 17 - protein, 5 aa, synthetic construct
CMGRC

SEQ ID NO: 18 - protein, 5 aa, synthetic construct
PHSRN

SEQ ID NO: 19 - protein, 4 aa, synthetic construct
LHRD

SEQ ID NO: 20 - protein, 17 aa, synthetic construct
RPRAPARSASRPRRPVE

SEQ ID NO: 21 - protein, 16 aa, synthetic construct
GLRKRLRKFRNKIKEK

SEQ ID NO: 22 - protein, 12 aa, synthetic construct
PLILLRLLRGQF

SEQ ID NO: 23 - protein, 4 aa, synthetic construct
CAQK

SEQ ID NO: 24 - protein, 31 aa, synthetic construct
KDEPQRRSARLSAKPAPPKPEPKPKKAPAKK

SEQ ID NO: 25 - protein, 9 aa, synthetic construct
CGNKRTRGC

SEQ ID NO: 26 - protein, 5 aa, synthetic construct
CREKA

SEQ ID NO: 27 - protein, 9 aa, synthetic construct
CSNRDARRC

SEQ ID NO: 28 - protein, 4 aa, synthetic construct
AHNP

SEQ ID NO: 29 - protein, 12 aa, synthetic construct
YCDGFYACYMDV

SEQ ID NO: 30 - protein, 12 aa, synthetic construct
KQFSALPFNFYT

SEQ ID NO: 31 - protein, 14 aa, synthetic construct
PLGLAGGWGERDGS

SEQ ID NO: 32 - protein, 13 aa, synthetic construct
GGGGYDRVTIHPF

SEQ ID NO: 33 - protein, 7 aa, synthetic construct
VHSPNKK

SEQ ID NO: 34 - protein, 7 aa, synthetic construct
VHPKQHR

SEQ ID NO: 35 - protein, 19 aa, synthetic construct
VLTTGLPALISWIKRKRQQ

SEQ ID NO: 36 - protein, 7 aa, synthetic construct
NNSKSHT

SEQ ID NO: 37 - protein, 12 aa, synthetic construct
VHPKQHRAEEAK

SEQ ID NO: 38 - protein, 10 aa, synthetic construct
CNNSKSHTCC

SEQ ID NO: 39 - protein, 4 aa, synthetic construct
VHPK

SEQ ID NO: 40 - protein, 13 aa, synthetic construct
VHPKQHRGGSKGC

SEQ ID NO: 41 - protein, 9 aa, synthetic construct
CKGGRAKDC

SEQ ID NO: 42 - protein, 13 aa, synthetic construct
CGFECVRQCPERC

SEQ ID NO: 43 - protein, 4 aa, synthetic construct
KDEL

SEQ ID NO: 44 - protein, 6 aa, synthetic construct
SEKDEL

SEQ ID NO: 45 - protein, 17 aa, synthetic construct
MRYMILGLLALAAVCSA

SEQ ID NO: 46 - protein, 12 aa, synthetic construct
RLDPTSYLRTFW

SEQ ID NO: 47 - protein, 6 aa, synthetic construct
WYRGRL

SEQ ID NO: 48 - protein, 26 aa, synthetic construct
ACDCRGDCFCGGKLAKLAKKLAKLAK

SEQ ID NO: 49 - protein, 30 aa, synthetic construct
MSVLTPLLLRGLTGSARRLPVPRAKIHWLC

SEQ ID NO: 50 - protein, 4 aa, synthetic construct
GKRK

SEQ ID NO: 51 - protein, 15 aa, synthetic construct
DKLAKLAKKLAKLAK

SEQ ID NO: 52 - protein, 6 aa, synthetic construct
KKKRKV

SEQ ID NO: 53 - protein, 17 aa, synthetic construct
KRPAATKKAGQAKKKKL

SEQ ID NO: 54 - protein, 12 aa, synthetic construct
GRKKRRQRRRPQ

SEQ ID NO: 55 - protein, 8 aa, synthetic construct
RRRRRRRR

SEQ ID NO: 56 - protein, 16 aa, synthetic construct
RQIKIWFQNRRMKWKK

SEQ ID NO: 57 - protein, 21 aa, synthetic construct
GDIMGEWGNEIFGAIAAGFLG

SEQ ID NO: 58 - protein, 4 aa, synthetic construct
GALA

SEQ ID NO: 59 - protein, 30 aa, synthetic construct
WEAALAEALAEALAEHLAEALAEALEALAA

SEQ ID NO: 60 - protein, 7 aa, synthetic construct
FFLIPKG

SEQ ID NO: 61 - protein, 13 aa, synthetic construct
THRPPMWSPWVWP

SEQ ID NO: 62 - protein, 19 aa, synthetic construct
TFFYGGSRGKRNNFKTEEY

SEQ ID NO: 63 - protein, 16 aa, synthetic construct
FKESWREARGTRIERG

SEQ ID NO: 64 - protein, 36 aa, synthetic construct
MCMPCFTTDHQMARKCDDCCGGKGRGKCYGPQCLCR

SEQ ID NO: 65 - protein, 9 aa, synthetic construct
  MOD_RES 9: note = diaminopimelic acid
DLATEPALX

SEQ ID NO: 66 - protein, 7 aa, synthetic construct
GFTGFLS

SEQ ID NO: 67 - protein, 29 aa, synthetic construct
YTIWMPENPRPGTPCDIFTNSRGKRASNG

SEQ ID NO: 68 - protein, 11 aa, synthetic construct
CRGDKRGPDEC

SEQ ID NO: 69 - protein, 36 aa, synthetic construct
TAMRAVDKLLLHLKKLFREGQFNRNFESIIICRDRT

SEQ ID NO: 70 - protein, 9 aa, synthetic construct
CGEMGWVRC

SEQ ID NO: 71 - protein, 12 aa, synthetic construct
  MOD_RES 1: note = 3,4-Dihydroxyphenylacetic acid
XMYIEALDKYAC

SEQ ID NO: 72 - protein, 7 aa, synthetic construct
PKKKRKV

SEQ ID NO: 73 - protein, 10 aa, synthetic construct
AAFEDLRVLS

SEQ ID NO: 74 - protein, 16 aa, synthetic construct
  MOD_RES 11: note = Qln
KRPAATKKRGXRKKKK

SEQ ID NO: 75 - protein, 8 aa, synthetic construct
GPLGIAGQ

SEQ ID NO: 76 - protein, 12 aa, synthetic construct
THRPPMWSPVWP

SEQ ID NO: 77 - protein, 12 aa, synthetic construct
SFHQFARATLAS

SEQ ID NO: 78 - protein, 12 aa, synthetic construct
YEQDPWGVKWWY

SEQ ID NO: 79 - protein, 9 aa, synthetic construct
CSPGAKVRC

SEQ ID NO: 80 - protein, 12 aa, synthetic construct
MEWSLEKGYTIK

SEQ ID NO: 81 - protein, 5 aa, synthetic construct
CALNN

SEQ ID NO: 82 - protein, 5 aa, synthetic construct
CCVVT

SEQ ID NO: 83 - protein, 6 aa, synthetic construct
CLPFFD

SEQ ID NO: 84 - protein, 7 aa, synthetic construct
  MOD_RES 1: note = gamma-Glu
  MOD_RES 3: note = gamma-Glu
  MOD_RES 5: note = gamma-Glu
XCXCXCG

SEQ ID NO: 85 - protein, 15 aa, synthetic construct
GCGGCGGKGGCGGCG

SEQ ID NO: 86 - protein, 21 aa, synthetic construct
GNYTCEVTELTREGETIIELK

SEQ ID NO: 87 - RNA, 534 nt, synthetic construct
  source 1..534: mol_type = other RNA
atggaaatctgcagaggcctccgcagtcacctaatcactctcctcctcttcctgttccattcagagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtag

SEQ ID NO: 88 - RNA, 465 nt, synthetic construct
  source 1..465: mol_type = other RNA
atgcggcctagcggcagaaagtctagcaagatgcaggcctttagaatctgggacgttaatcagaaaaccttctacctcagaaacaaccagctggtcgccggctacctgcagggccccaacgtgaatctggaagagaagatcgacgtggtgccaatcgagcctcacgccctgtttctgggcatccacggcggaaaaatgtgcctgtcttgtgtgaagtccggagatgagacaagactgcaactggaagccgtgaacattacagacctgagcgagaaccggaagcaggacaagcggttcgccttcatcagaagcgacagcggccctacaaccagcttcgagagcgccgcttgccccggctggttcctgtgcaccgccatggaagctgatcagcctgtgtccctgaccaacatgcctgatgaaggcgtgatggtgaccaagttctacttccaggaggacgagatgtga

SEQ ID NO: 89 - protein, 8 aa, synthetic construct
GPLGOAGQ

SEQ ID NO: 90 - protein, 10 aa, synthetic construct
GDEVEAPKGC